(19) United States
(12) Patent Application Publication
(10) Pub. No.: US 2008/0293696 A1
(43) Pub. Date: Nov. 27, 2008

(54) 2-Aminoarylcarboxamides Useful as Cancer Chemotherapeutic Agents

(75) Inventors: Catherine Brennan, Durham, NC (US); Julie A. Dixon, Bethany, CT (US); William J. Scott, Guilford, CT (US); Aniko Redman, Derby, CT (US); Benjamin D. Jones, New Haven, CT (US); Barton Phillips, New Haven, CT (US); Philip Wickens, Wallingford, CT (US); Istvan Enyedy, Milton, MA (US); Ellalahewage Kumarasinghe, Hamden, CT (US); Charles Kreiman, Wallingford, CT (US); Jacques Dumas, Bethany, CT (US); Uday Khire, Orange, CT (US); Chih-Yuan Chuang, San Mateo, CA (US); Harold C.E. Kluender, Trumbull, CT (US); Zhenqiu Hong, Milford, CT (US); Lei Wang, New Haven, CT (US); Donald Bierer, Bethany, CT (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Pharmaceuticals Corporation, West Haven, CT (US)

(21) Appl. No.: 11/630,850

(22) PCT Filed: Jun. 23, 2005
(86) PCT No.: PCT/US05/22518

§ 371 Date: Dec. 22, 2006

Related U.S. Application Data
(60) Provisional application No. 60/582,326, filed on Jun. 23, 2004.

(51) Int. Cl. A61K 31/55; C07D 405/12; A61K 31/4433; A61K 31/444; A61P 35/00; C07D 405/14; A61K 31/506

(52) U.S. Cl. 514/217.04; 546/282.4; 514/338; 514/333; 546/256; 544/333; 514/256; 540/597

(57) ABSTRACT

A compound having the formula (1)

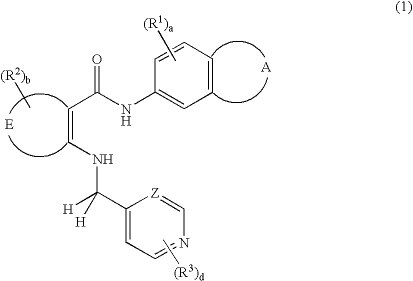

in which the ring containing E is a phenyl, a pyridine, or a pyrimidine. In formula (1) the symbol A represents

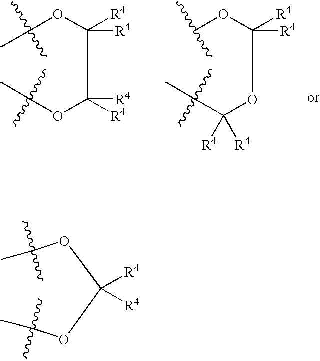

wherein the group R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring. Z represents N or CH when E forms a phenyl ring, and represents CH when E forms a pyridine or pyrimidine. The groups R¹, R² and R³ and the subscripts a, b, and d are as defined in the text and claims. Pharmaceutical compositions containing a compound of formula (1) and methods of treating cancer using compounds of formula (1) are also disclosed and claimed.

### FIELD

This invention relates to novel 2-aminoarylcarboxamide compounds, pro-drugs thereof, pharmaceutical compositions containing such compounds and pro-drugs, and the use of those compounds or compositions as cancer chemotherapeutic agents.

### BACKGROUND

Many disease conditions are known to be associated with deregulated angiogenesis. Among these are retinopathies; chronic inflammatory disorders including arthritis; arteriosclerosis; atherosclerosis; macular degeneration; and neoplastic diseases such as cancer. In recent years, much work has been carried out to find inhibitors of angiogenesis, in hopes of developing treatments for such disorders.

U.S. Pat. No. 6,448,277 (Novartis) discloses and claims certain benzamide derivatives for inhibition of VEGF receptor tyrosine kinase, tumor growth, and VEGF-dependent cell proliferation. WO 01/85715 (Novartis) relates to aza and polyazaanthranyl amides for use as medicaments for treating diseases caused by persistent angiogenesis. WO 03/040102 (Novartis) relates to anthranilic acid amides and their use as VEGF receptor tyrosine kinase inhibitors. U.S. Pat. No. 6,624,174 (Novartis) relates to 2-amino-nicotinamide derivatives and their use as VEGF-receptor tyrosine kinase inhibitors.

Published PCT application WO 02/066470 (Amgen) broadly discloses heterocycles containing amido and amino substituent groups, for prophylaxis and treatment of angiogenesis-mediated diseases. Published PCT application WO 2004/005279 (Amgen) discloses certain substituted anthranilic amide derivatives for the prophylaxis and treatment of angiogenesis-mediated diseases. Published PCT application WO 2004/007458 (Amgen) relates to substituted 2-alkylamine nicotinic amide derivatives and their uses in treatment of cancer and other disorders.

Published PCT application WO 00/27819 (Schering) discloses certain anthranilic acid amides for treatment of diseases that are triggered by angiogenesis. Published PCT application WO 02/090352 (Schering) relates to selective anthranilamide pyridine amides as inhibitors of VEGFR-2 and VEGFR-3. Published PCT application WO 01/81311 (Schering) relates to substituted benzoic acid amides and use thereof for the inhibition of angiogenesis.

Published PCT application WO 2004/063330 (OSI Pharmaceuticals) relates to (2-carboxamido)(3-amino)thiophene compounds for use in treatment of cancer.

Anthranilamides as angiogenesis inhibitors have been discussed in a series of research papers by scientists at Novartis and Schering. See Manley, et al., J. Med. Chem., 45, 5687-5693 (2002); Furet, et al., Bioorganic & Medicinal Chemistry Letters, 13, 2967-2971 (2003); Manley, et al., Cell. Mol. Biol. Lett., 8, 532-533 (2003); and Manley, et al., Biochimica et Biophysica Acta, 1697, 17-27 (2004).

### SUMMARY

The present invention relates to compounds having the formula (1)

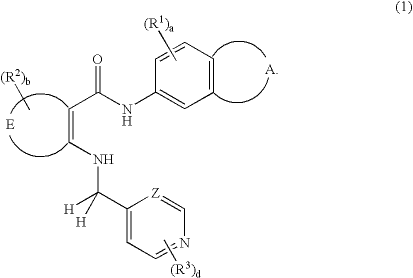

In this formula, the following definitions apply.

E represents

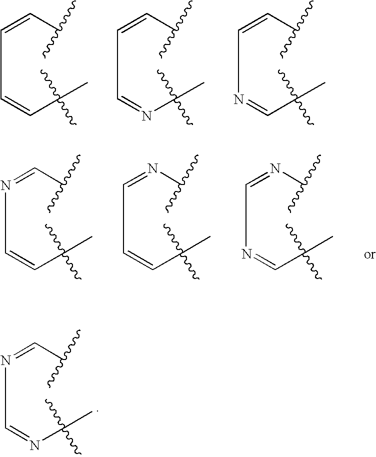

Z represents CH or N when E is

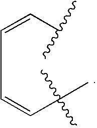

Z represents CH when E is

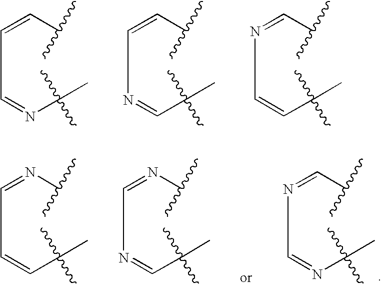

R¹ represents C₁₋₄ alkyl or halogen, and the subscript “a”, which represents the number of substituents R¹, is 0, 1 or 2.

R² represents C₁₋₄ alkyl, C₁₋₄ alkoxy, or halogen. The subscript “b”, which represents the number of substituents R², is 0, 1, 2, or 3 when E is

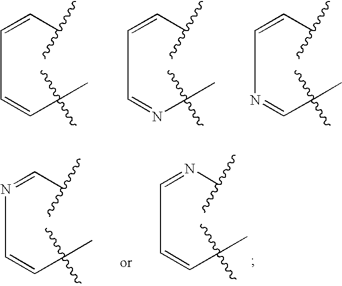

and b represents 0 or 1 when E is

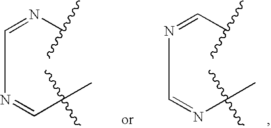

with the proviso that when b is 1 and E is

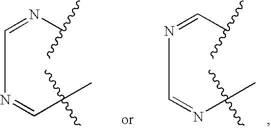

then the group R² is located adjacent to the amino or amido moiety, respectively, of formula (1).

R³ represents
1)

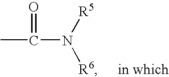

R⁵ represents H or C₁₋₄ alkyl which may optionally be substituted by OH or C₁₋₄ alkoxy; and
R⁶ represents
   H;
   C₃₋₆ cycloalkyl;
   —(CH₂)_{f}—O—(CH₂)_{g}—OR¹⁰ wherein R¹⁰ represents H or C₁₋₄ alkyl which is optionally substituted by F, and the subscripts f and g each independently represents 1, 2, or 3;

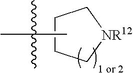

wherein the ring is optionally substituted by C₁₋₄ alkyl, and wherein R¹² represents H; C₁₋₄ alkyl which is optionally substituted by F; phenyl; benzyl; C₁₋₄ acyl; or SO₂R^{12a} in which R^{12a} represents C₁₋₄ alkyl which is optionally substituted by F; or
C₁₋₆ alkyl, which is optionally fluorinated up to perfluoro, or independently substituted by up to three substituents selected from:
   —SO₂R¹⁴ in which R¹⁴ represents C₁₋₅ alkyl which is optionally substituted by C₁₋₄ alkoxy or F;

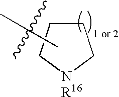

therein the ring is optionally substituted by C₁₋₄ alkyl; and wherein R¹⁶ represents H; C₁₋₄ alkyl optionally substituted by F; phenyl; benzyl; C₁₋₄ acyl; or SO₂R^{16a} in which R^{16a} represents C₁₋₄ alkyl optionally substituted by F;

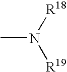

wherein R¹⁸ and R¹⁹ each independently represents H; C₁₋₅ alkyl; or phenyl which is optionally substituted by C₁₋₄ alkyl, halogen, OH, C₁₋₄ alkoxy, C₁₋₄ acyl, or SO₂R^{19a} in which R^{19a} represents C₁₋₄ alkyl optionally substituted by F;

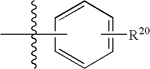

wherein R²⁰ represents from 0 to 4 optional substituents independently selected from C₁₋₄ alkyl, OH, C₁₋₄ alkoxy, halogen, NO₂, CN, and morpholino;

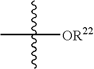

wherein R²² represents H or C₁₋₅ alkyl which is optionally substituted by F,

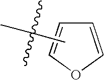

which is optionally substituted by C₁₋₄ alkyl;

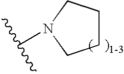

which is optionally substituted by C₁₋₄ alkoxy, F, or C₁₋₄ alkyl which is optionally substituted by F;

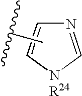

in which the ring is optionally substituted by C₁₋₄ alkyl or halogen, and R²⁴ represents H or C₁₋₄ alkyl which is optionally substituted by F;

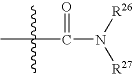

wherein R²⁶ and R²⁷ independently represent H or C₁₋₂ alkyl optionally substituted by F;

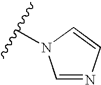

which is optionally substituted by halogen or by C₁₋₄ alkyl optionally substituted by F;
—CO₂R²⁸ wherein R²⁸ represents C₁₋₄ alkyl;

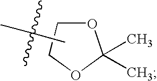

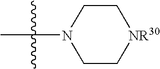

wherein the ring is optionally substituted by C₁₋₄ alkyl, C₁₋₄ alkoxy, or F; and wherein R³⁰ represents H; C₁₋₄ alkyl optionally substituted by F; phenyl; C₁₋₄ acyl; or SO₂R³¹ wherein R³¹ represents C₁₋₂ alkyl optionally substituted by F;

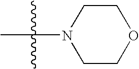

optionally substituted by F or C₁₋₄ alkyl;

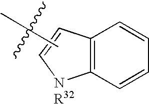

wherein R³² represents H or C₁₋₄ alkyl optionally substituted by F, and the phenyl ring is optionally substituted by C₁₋₄ alkyl, C₁₋₄ alkoxy, or halogen;

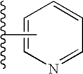

which is optionally substituted by C₁₋₄ alkyl or C₁₋₄ alkoxy;

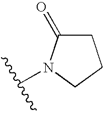

which is optionally substituted by C₁₋₄ alkyl, C₁₋₄ alkoxy, or F;
—SR³⁴ wherein R³⁴ represents H or C₁₋₄ alkyl;

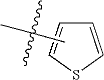

which is optionally substituted by C₁₋₄ alkyl or halogen;

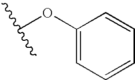

which is optionally substituted by halogen, OH, C₁₋₄ alkoxy, or C₁₋₄ alkyl which is optionally substituted by F;
2)

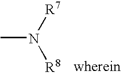

R⁷ represents H or C₁₋₄ alkyl;
R⁸ represents
   H;
   —(CH₂)ₕ—O—(CH₂)ᵢ—OR³⁶ wherein
      R³⁶ represents H or C₁₋₄ alkyl which is optionally substituted by F, and subscripts h and i are independently 1, 2, or 3;

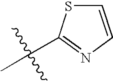

which is optionally substituted by halogen or by C₁₋₄ alkyl which is optionally substituted by F; or
C₁₋₆ alkyl which is optionally substituted by up to three substituents selected from:
   OH;
   C₁₋₄ alkoxy;

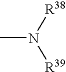

wherein R³⁸ and R³⁹ independently represent H, C₁₋₄ alkyl, C₁₋₄ acyl, or SO₂R^{39a}, and R^{39a} represents C₁₋₄ alkyl optionally substituted by F;

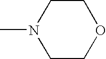

which is optionally substituted by F or C₁₋₄ alkyl;

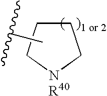

wherein the ring is optionally substituted by F, C₁₋₄ alkyl, or C₁₋₄ alkoxy; and R⁴⁰ represents H, C₁₋₄ acyl, C₁₋₄ alkyl which is optionally substituted by F, or SO₂R^{40a} wherein R^{40a} represents C₁₋₄ alkyl which is optionally substituted by F;

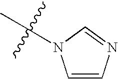

which is optionally substituted by halogen or C₁₋₄ alkyl which is optionally substituted by F;

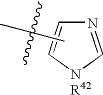

wherein the ring is optionally substituted by halogen, and R⁴² represents H or C₁₋₄ alkyl which is optionally substituted by F;

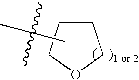

which is optionally substituted by F or C₁₋₄ alkyl; and

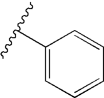

which is optionally substituted by halogen, C₁₋₄ alkoxy, or C₁₋₄ alkyl which is optionally substituted by F;
3) —CN;
4) -halogen;
5) —C₁₋₄ alkyl which is optionally substituted by OH or C₁₋₄ alkoxy;
6)

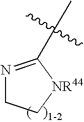

wherein the ring is optionally substituted by F, C₁₋₄ alkoxy, or C₁₋₄ alkyl, and R⁴⁴ represents H or C₁₋₄ alkyl optionally substituted by F;
7)

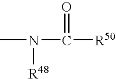

wherein
   R⁴⁸ represents H, C₁₋₂ alkyl, or C(O)—(CH₂)₁₋₃—CO₂R^{48a} wherein
      R^{48a} represents H or C₁₋₄ alkyl; and
   R⁵⁰ represents
      —(CH₂)ⱼ—O—(CH₂)ₖ—R^{50a} wherein R^{50a} represents OH, C₁₋₄ alkoxy, or C₁₋₄ alkoxycarbonyl; and subscripts j and k are independently 1, 2, or 3;
   or R⁵⁰ represents
      C₁₋₄ alkyl optionally substituted by C₁₋₂ alkoxy, C₁₋₄ acyloxy, or C₁₋₄ alkoxycarbonyl;
8)

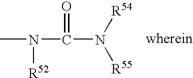

R⁵² represents H or C₁₋₂ alkyl;
R⁵⁴ represents H or C₁₋₄ alkyl; and
R⁵⁵ represents H; C₁₋₄ alkyl optionally substituted by F or C₁₋₄ alkoxy; phenyl optionally substituted by CN, OH, C₁₋₄ alkoxy, or C₁₋₄ alkyl;

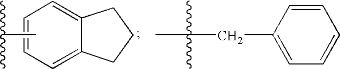

wherein the ring is optionally substituted by C₁₋₄ alkyl, C₁₋₄ alkoxy, or OH; or R⁵⁵ represents

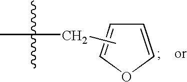

R⁵⁴ and R⁵⁵ are optionally joined and together with the N atom to which they are attached form a 5- or 6-membered saturated heterocycle selected from pyrrolidinyl, morpholinyl, thiomorpholinyl, and piperizinyl optionally substituted on N with C₁₋₄ alkyl; or
9)

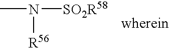

R⁵⁶ represents H or C₁₋₂ alkyl; and
R⁵⁸ represents C₁₋₄ alkyl which is optionally substituted by F; or represents phenyl which is optionally substituted by halogen, C₁₋₄ alkyl, or C₁₋₄ alkoxy.
The subscript d, which represents the number of substituents R³, is 0 or 1.

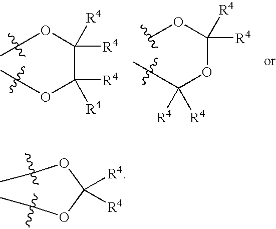

A represents
R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring.
A further proviso is that any R³ group is located adjacent to a ring nitrogen atom.
A pharmaceutically acceptable salt or stereoisomer of this compound is also within the scope of the invention.

The invention also relates to pharmaceutical compositions which comprise a compound of formula (1) as defined above plus a pharmaceutically acceptable carrier.

In addition, the invention relates to a method of treating cancer comprising administering to a subject in need thereof an effective amount of a compound of formula (1) as defined above.

### DETAILED DESCRIPTION

In a first embodiment, the present invention relates to a compound having the formula (I)

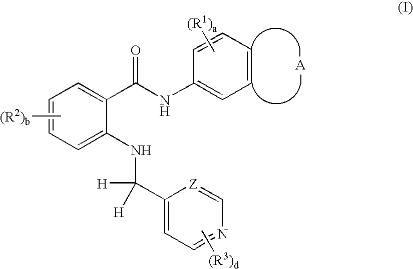

In this formula, the following definitions apply.

Z represents CH or N.

R¹ represents C₁₋₄ alkyl or halogen, and the subscript “a”, which represents the number of substituent groups R¹, is 0, 1 or 2.

R² represents C₁₋₄ alkyl, C₁₋₄ alkoxy, or halogen, and the subscript “b”, which represents the number of substituent groups R², is 0, 1, 2, or 3.

R³ represents
1)

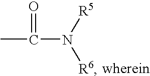

R⁵ represents H or C₁₋₄ alkyl which may optionally be substituted by OH or C₁₋₄ alkoxy; and
R⁶ represents
   H;
   C₃₋₆ cycloalkyl;
   —(CH₂)_{f}—O—(CH₂)_{g}—OR¹⁰ wherein R¹⁰ represents H or C₁₋₄ alkyl which is optionally substituted by F, and the subscripts f and g each independently represents 1, 2, or 3;

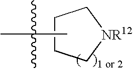

wherein the ring is optionally substituted by C₁₋₄ alkyl, and wherein R¹² represents H; C₁₋₄ alkyl which is optionally substituted by F; phenyl; benzyl; C₁₋₄ acyl; or SO₂R^{12a} in which R^{12a} represents C₁₋₄ alkyl which is optionally substituted by F; or
C₁₋₆ alkyl, which is optionally fluorinated up to perfluoro, or independently substituted by up to three substituents selected from:
   —SO₂R¹⁴ in which R¹⁴ represents C₁₋₅ alkyl which is optionally substituted by C₁₋₄ alkoxy or F;

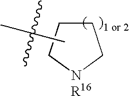

wherein the ring is optionally substituted by C₁₋₄ alkyl; and wherein R¹⁶ represents H; C₁₋₄ alkyl optionally substituted by F; phenyl; benzyl; C₁₋₄ acyl; or SO₂R^{16a} in which R^{16a} represents C₁₋₄ alkyl optionally substituted by F;

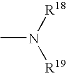

wherein R¹⁸ and R¹⁹ each independently represents H; C₁₋₅ alkyl; or phenyl which is optionally substituted by C₁₋₄ alkyl, halogen, OH, C₁₋₄ alkoxy, C₁₋₄ acyl, or SO₂R^{19a} in which R^{19a} represents C₁₋₄ alkyl optionally substituted by F;

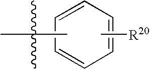

wherein R²⁰ represents from 0 to 4 optional substituents independently selected from C₁₋₄ alkyl, OH, C₁₋₄ alkoxy, halogen, NO₂, CN, and morpholino;

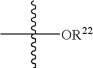

wherein R²² represents H or C₁₋₅ alkyl which is optionally substituted by F,

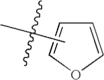

which is optionally substituted by C₁₋₄ alkyl;

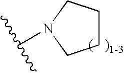

which is optionally substituted by C₁₋₄ alkoxy, F, or C₁₋₄ alkyl which is optionally substituted by F;

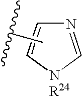

in which the ring is optionally substituted by C₁₋₄ alkyl or halogen, and R²⁴ represents H or C₁₋₄ alkyl which is optionally substituted by F;

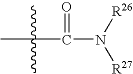

wherein R²⁶ and R²⁷ independently represent H or C₁₋₂ alkyl optionally substituted by F;

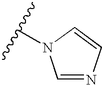

which is optionally substituted by halogen or by C₁₋₄ alkyl optionally substituted by F;
—CO₂R²⁸ wherein R²⁸ represents C₁₋₄ alkyl;

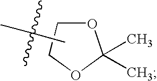

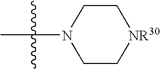

wherein the ring is optionally substituted by C₁₋₄ alkyl, C₁₋₄ alkoxy, or F; and wherein R³⁰ represents H; C₁₋₄ alkyl optionally substituted by F; phenyl; C₁₋₄ acyl; or SO₂R³¹ wherein R³¹ represents C₁₋₂ alkyl optionally substituted by F;

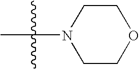

optionally substituted by F or C₁₋₄ alkyl;

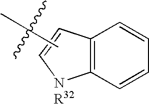

wherein R³² represents H or C₁₋₄ alkyl optionally substituted by F, and the phenyl ring is optionally substituted by C₁₋₄ alkyl, C₁₋₄ alkoxy, or halogen;

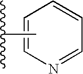

which is optionally substituted by C₁₋₄ alkyl or C₁₋₄ alkoxy;

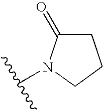

which is optionally substituted by C₁₋₄ alkyl, C₁₋₄ alkoxy, or F;
—SR³⁴ wherein R³⁴ represents H or C₁₋₄ alkyl;

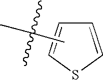

which is optionally substituted by C₁₋₄ alkyl or halogen;

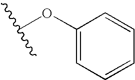

which is optionally substituted by halogen, OH, C₁₋₄ alkoxy, or C₁₋₄ alkyl which is optionally substituted by F.
2)

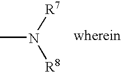

R⁷ represents H or C₁₋₄ alkyl;
R⁸ represents
   H;
   —(CH₂)ₕ—O—(CH₂)ᵢ—OR³⁶ wherein
      R³⁶ represents H or C₁₋₄ alkyl which is optionally substituted by F, and subscripts h and i are independently 1, 2, or 3;

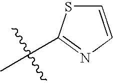

which is optionally substituted by halogen or by C₁₋₄ alkyl which is optionally substituted by F; or
C₁₋₆ alkyl which is optionally substituted by up to three substituents selected from:
   OH;
   C₁₋₄ alkoxy;

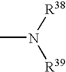

wherein R³⁸ and R³⁹ independently represent H, C₁₋₄ alkyl, C₁₋₄ acyl, or SO₂R^{39a}, and R^{39a} represents C₁₋₄ alkyl optionally substituted by F;

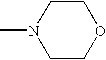

which is optionally substituted by F or C₁₋₄ alkyl;

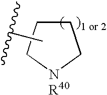

wherein the ring is optionally substituted by F, C₁₋₄ alkyl, or C₁₋₄ alkoxy; and R⁴⁰ represents H, C₁₋₄ acyl, C₁₋₄ alkyl which is optionally substituted by F, or SO₂R^{40a} wherein R^{40a} represents C₁₋₄ alkyl which is optionally substituted by F;

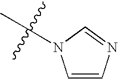

which is optionally substituted by halogen or C₁₋₄ alkyl which is optionally substituted by F;

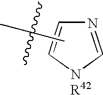

wherein the ring is optionally substituted by halogen, and R⁴² represents H or C₁₋₄ alkyl which is optionally substituted by F;

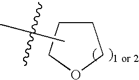

which is optionally substituted by F or C₁₋₄ alkyl; and

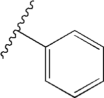

which is optionally substituted by halogen, C₁₋₄ alkoxy, or C₁₋₄ alkyl which is optionally substituted by F;
3) —CN;
4) -halogen;
5) —C₁₋₄ alkyl which is optionally substituted by OH or C₁₋₄ alkoxy;
6)

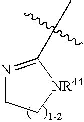

wherein the ring is optionally substituted by F, C₁₋₄ alkoxy, or C₁₋₄ alkyl, and R⁴⁴ represents H or C₁₋₄ alkyl optionally substituted by F;
7)

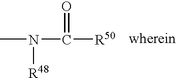

R⁴⁸ represents H, C₁₋₂ alkyl, or C(O)—(CH₂)₁₋₃—CO₂R^{48a} wherein
   R^{48a} represents H or C₁₋₄ alkyl; and
R⁵⁰ represents
   —(CH₂)ⱼ—O—(CH₂)ₖ—R^{50a} wherein
      R^{50a} represents OH, C₁₋₄ alkoxy, or C₁₋₄ alkoxycarbonyl; and subscripts j and k are independently 1, 2, or 3;
   or R⁵⁰ represents
      C₁₋₄ alkyl optionally substituted by C₁₋₂ alkoxy, C₁₋₄ acyloxy, or C₁₋₄ alkoxycarbonyl;
8)

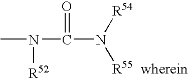

R⁵² represents H or C₁₋₂ alkyl;
R⁵⁴ represents H or C₁₋₄ alkyl; and
R⁵⁵ represents H; C₁₋₄ alkyl optionally substituted by F or C₁₋₄ alkoxy; phenyl optionally substituted by CN, OH, C₁₋₄ alkoxy, or C₁₋₄ alkyl;

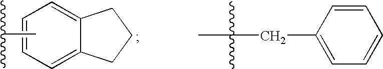

wherein the ring is optionally substituted by C₁₋₄ alkyl, C₁₋₄ alkoxy, or OH; or R⁵⁵ represents

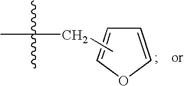

R⁵⁴ and R⁵⁵ are optionally joined and together with the N atom to which they are attached form a 5- or 6-membered saturated heterocycle selected from pyrrolidinyl, morpholinyl, thiomorpholinyl, and piperizinyl optionally substituted on N with C₁₋₄ alkyl;
9)

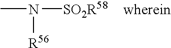

R⁵⁶ represents H or C₁₋₂ alkyl; and
R⁵⁸ represents C₁₋₄ alkyl which is optionally substituted by F; or represents phenyl which is optionally substituted by halogen, C₁₋₄ alkyl, or C₁₋₄ alkoxy.
The subscript d, which represents the number of substituents R³, is 0 or 1.
A represents

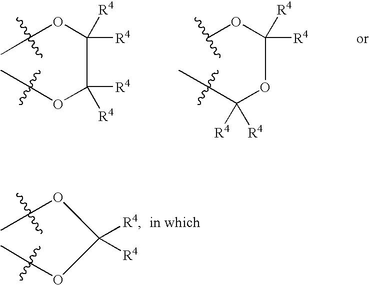

R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring.
A further proviso is that any R³ group is located adjacent to a ring nitrogen atom.
A pharmaceutically acceptable salt or stereoisomer of this compound is also within the scope of the invention.

The invention also relates to pharmaceutical compositions which comprise a compound of formula (1) as defined above plus a pharmaceutically acceptable carrier.

In addition, the invention relates to a method of treating cancer comprising administering to a subject in need thereof an effective amount of a compound of formula (I) as defined above.

In a second embodiment, the present invention relates to a compound having the formula (II)

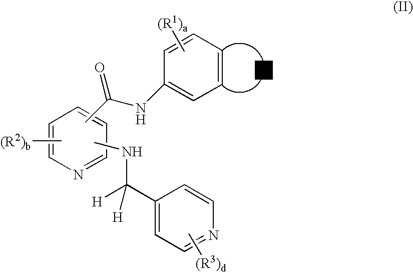

In this formula, the following definitions apply.

R¹ represents C₁₋₄ alkyl or halogen, and the subscript a, which represents the number of substituents R¹, is 0, 1 or 2.

R² represents C₁₋₄ alkyl, C₁₋₄ alkoxy, or halogen; and the subscript b, which represents the number of substituents R², is 0, 1, 2, or 3. Preferably, R² represents C₁₋₄ alkyl or halogen, and most preferably represents halogen.

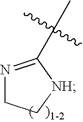

R³ represents —C(O)NR⁵R⁶; —NR⁷R⁸; —CN; -halogen; —C₁₋₄ alkyl; or and the subscript d, which represents the number of substituents R³, is 0 or 1. Preferably, R³ represents —C(O)NR⁵R⁶; —NR⁷R⁸; or —C₁₋₄ alkyl; and most preferably, R³ represents —C(O)NR⁵R⁶; or —NR⁷R⁸.

A represents

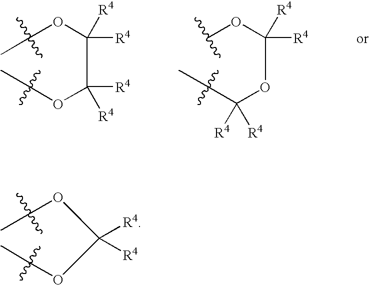

R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring.

The groups R⁵ and R⁶ each independently represents H, C₁₋₄ alkyl, or —C₁₋₄-alkyl-C₁₋₂-alkoxy. The groups R⁷ and R⁸ each independently represents H or C₁₋₄ alkyl.

In addition, in the compounds of the invention, any R³ group is located adjacent to the ring nitrogen atom, and the amido and amino side chains on the central pyridine ring are located adjacent to each other.

A pharmaceutically acceptable salt or stereoisomer of this compound is also within the scope of the invention.

The invention also relates to pharmaceutical compositions which comprise a compound of formula (II) as defined above plus a pharmaceutically acceptable carrier.

In addition, the invention relates to a method of treating cancer comprising administering to a subject in need thereof an effective amount of a compound of formula (II) as defined above.

In a third embodiment, the present invention relates to a compound having formula (III) or formula (IV)

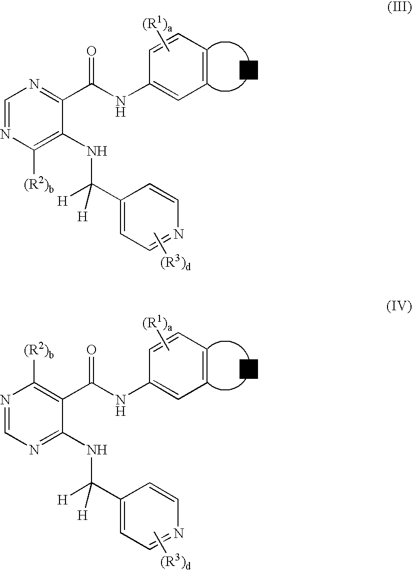

In this formula, the following definitions apply.

R¹ represents C₁₋₄ alkyl or halogen, and the subscript a, which represents the number of substituents R¹, is 0, 1 or 2.

R² represents C₁₋₄ alkyl, C₁₋₄ alkoxy, or halogen; and the subscript b, which represents the number of substituents R², is 0 or 1. Preferably, R² represents C₁₋₄ alkyl or halogen, and most preferably represents halogen.

R³ represents —C(O)NR⁵R⁶; —NR⁷R⁸; —CN; -halogen; —C₁₋₄ alkyl; or

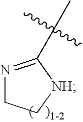

and the subscript d, which represents the number of substituents R³, is 0 or 1. Preferably, R³ represents —C(O)NR⁵R⁶; —NR⁷R⁸; or —C₁₋₄ alkyl; and most preferably, R³ represents —C(O)NR⁵R⁶; or NR⁷R⁸.

A represents

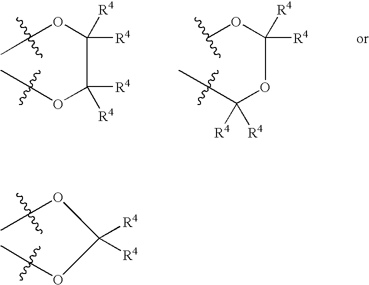

R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring.

The groups R⁵ and R⁶ each independently represents H, C₁₋₄ alkyl, or —C₁₋₄-alkyl-C₁₋₂-alkoxy.

The groups R⁷ and R⁸ each independently represents H or C₁₋₄ alkyl.

In addition, in the compounds of the invention, any R³ group is located adjacent to the ring nitrogen atom.

A pharmaceutically acceptable salt or stereoisomer of this compound is also within the scope of the invention.

The invention also relates to pharmaceutical compositions which comprise a compound of formula (III) or formula (IV) as defined above plus a pharmaceutically acceptable carrier.

In addition, the invention relates to a method of treating cancer comprising administering to a subject in need thereof an effective amount of a compound of formula (III) or formula (IV) as defined above.

### DEFINITIONS

The terms identified above have the following meanings throughout:

The terms “halogen” and “halo” mean Cl, Br, F and I, where Cl, Br and F are preferred.

The term “C₁₋₆ alkyl” means a linear or branched saturated hydrodarboncarbon moiety typically having from 1 to 6 carbon atoms, and preferably having from one to 4 carbon atoms. Such groups include but are not limited to methyl, ethyl, n-propyl, isopropyl, n-butyl, isobutyl, sec-butyl, tert-butyl, pentyl, hexyl, and the like.

The terms “C₁₋₂ alkoxy and C₁₋₄ alkoxy” mean a linear or branched saturated hydrocarbon group having from 1 to 2, or from 1 to 4 carbon atoms, respectively, said group being attached to an O atom. The O atom is the point of attachment of the alkoxy substituent to the rest of the molecule. Such groups include but are not limited to methoxy, ethoxy, n-propoxy, isopropoxy, and the like.

The term “—C₁₋₄ alkyl-C₁₋₂ alkoxy” means a C₁₋₄ alkyl in which a H atom on any C atom in the group is replaced by a C₁₋₂ alkoxy group. Such groups include but are not limited to methoxymethyl, ethoxymethyl, 2-methoxyethyl, 4-ethoxybutyl and the like.

In the description and claims relating to the compounds of the invention, various groups are stated to be “optionally substituted”, the number of such substituents not being stated. It is to be understood that in principle, the number of such “optional” substituents may be up to the number of available valences, although in general, the number of substituents will be 1 or 2, and more generally, 1. The skilled in the art are also aware that certain combinations of chemical groups are not desirable, and such undesirable combinations include substitutions in which a single carbon is attached to two oxygens, or one oxygen and a halogen, or two sulfur atoms, or two nitrogen atoms.

The compounds of this invention may contain one or more asymmetric centers, depending upon the location and nature of the various substituents desired. Asymmetric carbon atoms may be present in the (R) or (S) configuration. It is intended that all such possible stereoisomers (including enantiomers and diastereomers) are included within the scope of the present invention. Preferred compounds are those with the absolute configuration of the compound of this invention which exhibits the more desirable biological activity. Separated, pure or partially purified stereoisomers or racemic mixtures of the compounds of this invention are also included within the scope of the present invention. The purification of said stereoisomers and the separation of said stereoisomeric mixtures can be accomplished by standard techniques known in the art.

Pharmaceutically acceptable salts of these compounds are also within the scope of this invention. The term “pharmaceutically acceptable salt” refers to a relatively non-toxic, inorganic or organic salt of a compound of the present invention. For example, see S. M. Berge, et al. “Pharmaceutical Salts,” J. Pharm. Sci., 66: 1-19, 1977.

Representative salts of the compounds of this invention include the conventional non-toxic salts and the quaternary ammonium salts that are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates such as dimethyl, diethyl, and dibutyl sulfate; and diamyl sulfates, long chain halides such as decyl, lauryl, myristyl and strearyl chlorides, bromides and iodides, aralkyl halides such as benzyl and phenethyl bromides, and others.

Unless the context clearly indicates to the contrary, whenever the language “compounds of this invention,” “compounds of the present invention”, and the like, is used herein, it is intended to include the chemically feasible pharmaceutically acceptable salts, prodrugs such as esters as well as all stereoisomeric forms of the referenced compounds

### General Methods of Preparation

Compounds of formulae (I)-(IV) may be prepared by synthetic procedures known to those skilled in the art or by methods analogous thereto. These methods are summarized below in Reaction Schemes 1-16.

Reaction Schemes 1 and 2 illustrate general methods useful for the preparation of compounds of formula (I).

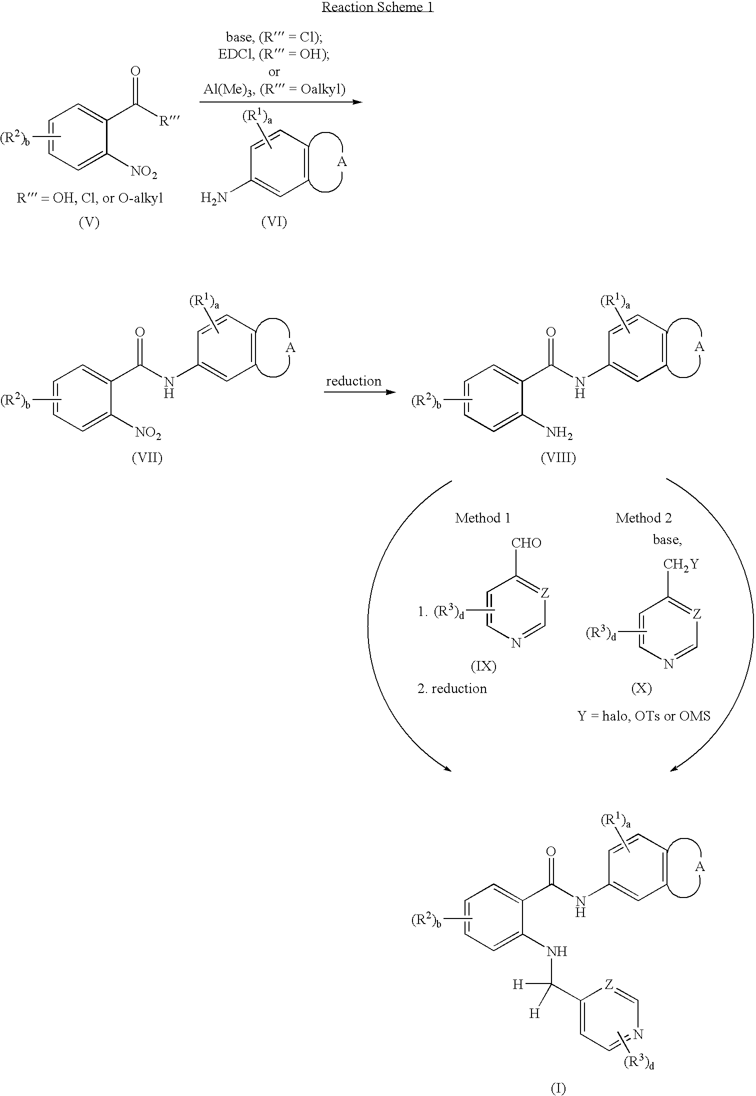

In Reaction Scheme 1, a 2-nitrobenzoic acid or derivative of formula (V) is allowed to react with an aromatic amine of formula (VI) to provide the 2-nitrobenzamide of formula (VII). Reduction of the nitro group in (VII) using for example, H₂/Pd—C catalyst, provides the 2-aminobenzamide of formula (VIII). The conversion of (VIII) to the formula (I) compound is carried out using either reductive amination using a pyridine or pyrimidine aldehyde of formula (IX) and a reducing agent such as sodium cyanoborohydride, or N-alkylation using a pyridine or pyrimidine methyl halide, tosylate or mesylate of formula (X) and a base.

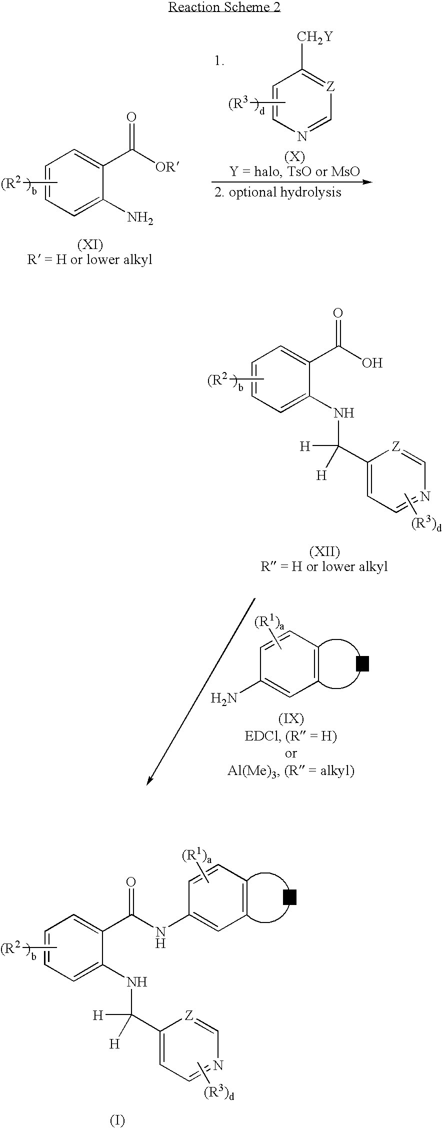

Reaction Scheme 2 provides an alternative method of preparation of the formula (I) compound starting from an anthranilic acid or anthranilic ester of formula (XI). Alkylation of this starting material with a pyridine or pyrimidine methyl halide, tosylate or mesylate of formula (X) in the presence of a base provides the intermediate of formula (XII), which is then allowed to react with an aromatic amine of formula (IX), giving the compound of formula (I).

Preparation of compounds of formula (II) is illustrated in Reaction Schemes 3 and 4 below.

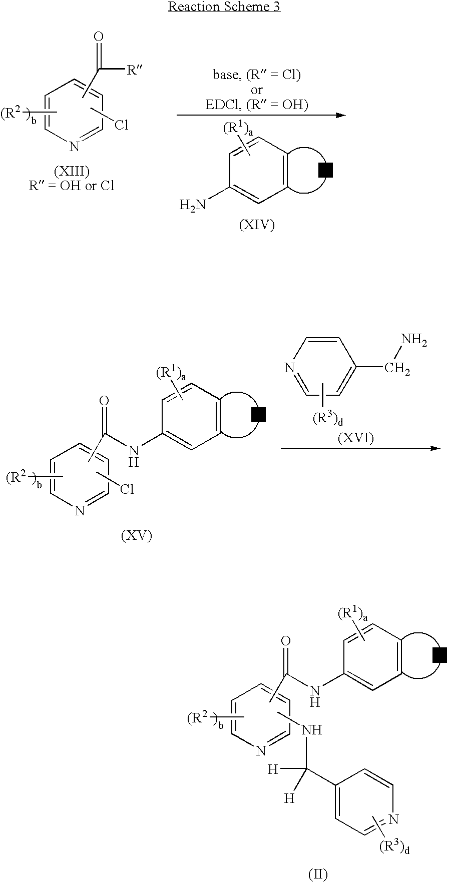

In Reaction Scheme 3, the compound of formula (XIII) is allowed to react with an aromatic amine of formula (XIV) in the presence of a base (when R″ is Cl) or EDCI (when R″ is OH) to give a chloroamide of formula (XV). This formula (XV) compound is then allowed to react with a 1-pyridin-4-ylmethanamine of formula (XVI), either in the presence of a high boiling inert solvent or neat, to give the product of formula (II).

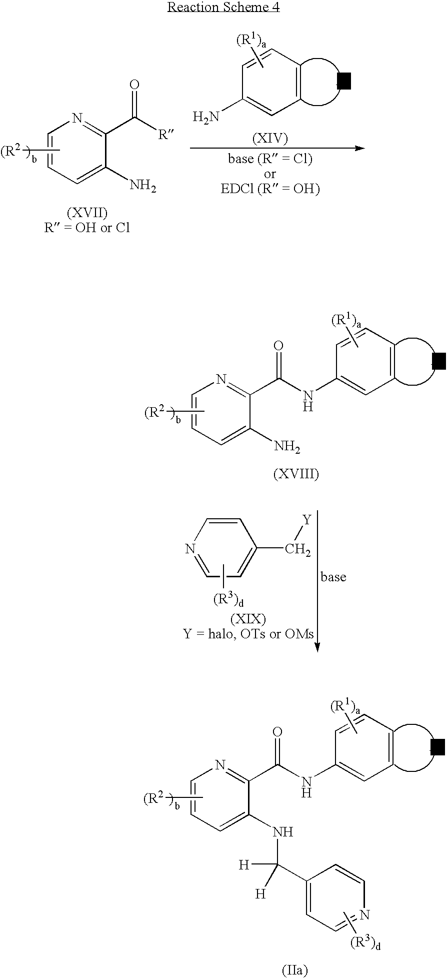

Reaction Scheme 4 shows the synthesis of compounds of formula (IIa), in which the amine side chain is attached at the 3-position of the central pyridine ring and the carboxamide side chain is attached at the 2-position of the central pyridine ring. The 3-aminopyridinecarboxylic acid or acid chloride of formula (XVII) is allowed to react with the aromatic amine of formula (XIV) in the presence of base (when R″=Cl) or a coupling agent such as EDCI (when R″=OH) to provide the pyridine carboxamide of formula (XVIII). N-alkylation of (XVIII) with a 1-pyridylmethyl halide, tosylate or mesylate of formula (XIX) in the presence of a base such as K₂CO₃, provides the compound of formula (IIa).

The preparation of compounds of formulae (III) and (IV), represented collectively as

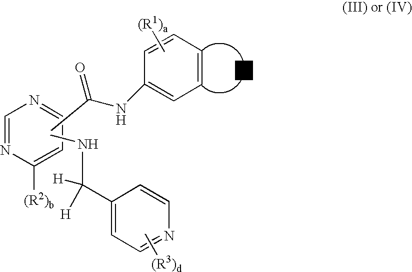

is illustrated in Reaction Scheme 5, starting from the appropriately substituted 2-alkylthio-halopyrimidine carboxylic acids or esters of formula (XX). In this sequence, the compound of formula (XX) more specifically represented by the compound of formula (XXa), is the starting material for preparation of the compound of formula (III). Likewise, the compound of formula (XX) more specifically represented by the compound of formula (XXb), is the starting material for the compound of formula (IV).

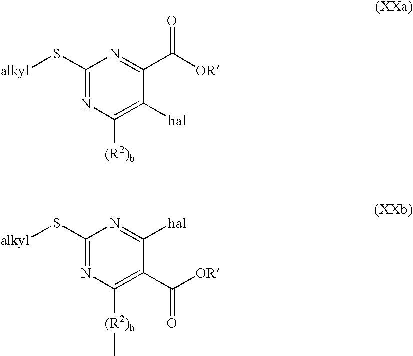

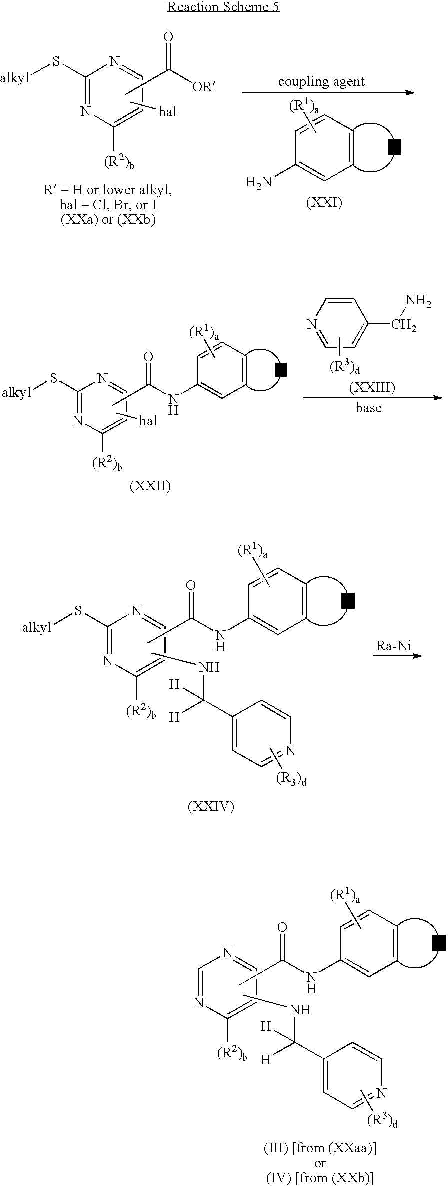

In Reaction Scheme 5, the compound of formula (XX) is allowed to react with an aromatic amine of formula (XXI) in the presence of a coupling agent such as EDCI (when R′=H) or Al(Me)₃ (when R′=alkyl) to give the amide of formula (XXII). A nucleophilic aromatic substitution reaction of (XXII) with the pyridine methylamine of formula (XXIII) in the presence of a base such as K₂CO₃ and CuO gives the intermediate of formula (XXIV). The 2-alkylthio group present in the compound of formula (XXIV) is removed by a Raney-Nickel desulfurization to give the compounds of formulae (III) or (IV).

### Synthetic Schemes for Intermediates

Starting materials of Formulae (IX), (X) and (VI) are commercially available (e.g., Lanxess, Germany) or may be prepared by standard means well known in the art, or as described in Reaction Schemes 6-12.

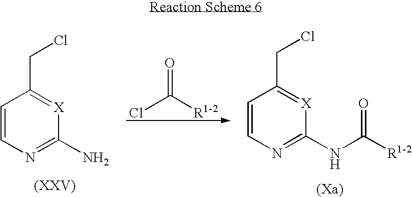

Compounds of Formula (Xa), which corresponds to

Formula (X) where R³ is

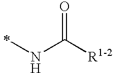

and Y is Cl, may be prepared as shown in Reaction Scheme 6 by reaction of an acid chloride with a chloromethyl heteroarylamine of Formula (XXV), generally in the presence of a base such as triethylamine.

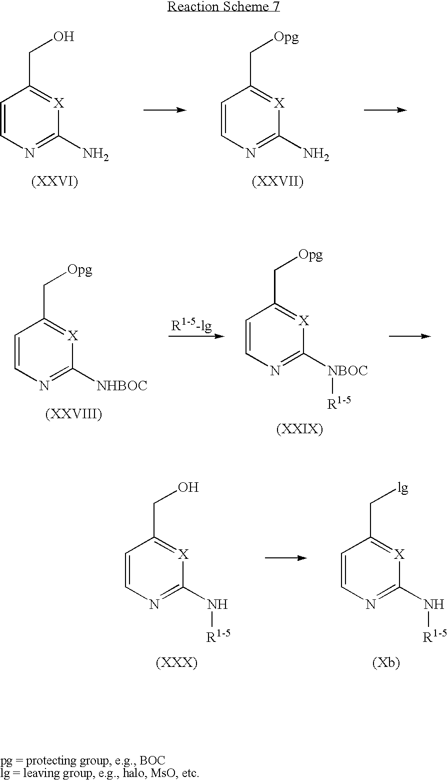

Compounds of Formula (Xb), which corresponds to Formula (X) where R³ is

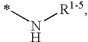

can be prepared as shown in Reaction Scheme 7 from hydroxymethylheteroaryl amines of Formula (XXVI). Protection of the alcohol and conversion to the BOC-derivative of Formula (XXVIII) is followed by N-alkylation to give the intermediate of Formula (XXIX). Deprotection of the alcohol and amine, followed by conversion of the hydroxy group to a leaving group, (for example, using SOCl₂, when 1 g is Cl) gives the intermediate of Formula (Xb).

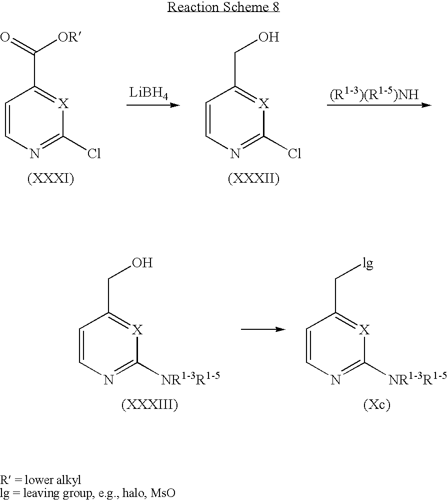

Compounds of Formula (Xc), which corresponds to Formula (X) where R³ is

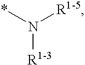

can be prepared by the route illustrated in Reaction Scheme 8. The chloroheteroarylcarboxylic acid derivative of Formula (XXXI) is reduced to the chloroheteroaryl alcohol of Formula (XXXII) with a standard reagent such as lithium borohydride. Reaction of the chloro compound with an amine of Formula (R¹⁻³)(R¹⁻⁵)NH gives the intermediate alcohol of Formula (XXXIII). Conversion of this alcohol to a leaving group, e.g. mesylate, completes the synthesis of the compound of Formula (Xc)

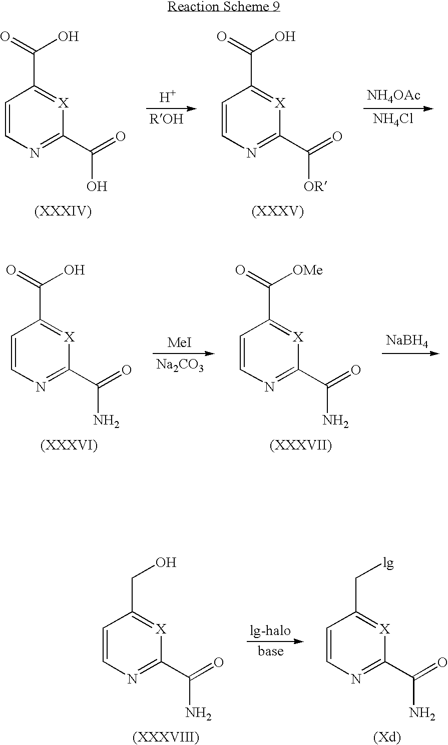

Compounds of Formula (Xd), which corresponds to Formula (X) where R³ is

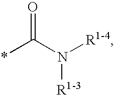

can be prepared as shown in Reaction Scheme 9 from the dicarboxylic acid of Formula (XXXIV) by conversion through the half acid ester (XXXV) to the acid amide of Formula (XXXVI). Esterification of (XXXVI) provides (XXXVII) which can be reduced with sodium borohydride to the alcohol (XXXVIII) and then converted to the Formula (Xd) compound, using for example MsCl and a base such as triethylamine.

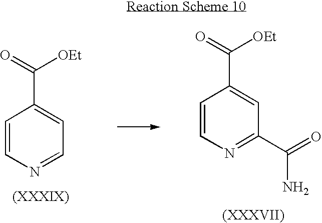

An alternate method of preparing the pyridine amide ester of Formula (XXXVII) is via the Minisci reaction shown in Reaction Scheme 10 in which the pyridine carboxylic acid ester is stirred in formamide with cooling to 10° C. in the presence of an equivalent of concentrated H₂SO₄, FeSO₄ and H₂O₂.

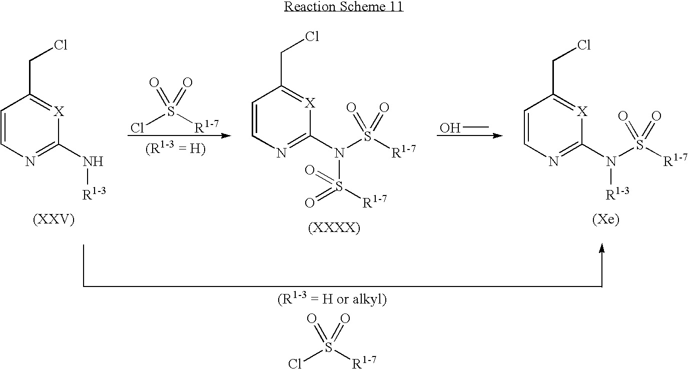

Compounds of Formula (Xe), which corresponds to Formula (X) where R³ is

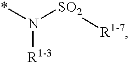

can be prepared by the route shown in Reaction Scheme 11. Starting from the intermediate of Formula (XXV), the sulfonamide (Xe) may be prepared in a manner analogous to that described for Formula (Xa), by reaction of (XXV) with a sulfonyl chloride in the presence of a base. The bis-sulfonylated compound (XXXX), if formed, may be converted to (Xe) if necessary, by reaction with aqueous base.

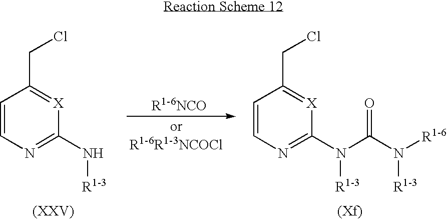

Compounds of Formula (Xf), which corresponds to Formula (X) where R³ is

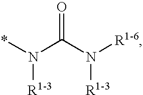

can be prepared by the route shown in Reaction Scheme 12. In the case that the R¹⁻³ on the right is H, the intermediate of Formula (XXV) is allowed to react with an isocyanate of Formula R¹⁻⁶NCO in an aprotic solvent such as dichloromethane. In the case that the R¹⁻³ on the right is alkyl, or that R¹⁻³ and R¹⁻⁶ are combined in a cyclic structure, the intermediate of Formula (XXV) is allowed to react with a carbamoyl chloride Formula R¹⁻⁶ R¹⁻³ NCOCl in an aprotic solvent such as dichloromethane in the presence of a base such as triethylamine or potassium carbonate. The use of a starting material of Formula (XXV) in which the R¹⁻³ on the left is alkyl results in the preparation of a urea of structure Xf where R³ is

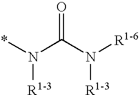

in which the R¹⁻³ group on the left is alkyl. In the case that both R¹⁻³ on the right and R¹⁻⁶ are H, benzoyl isocyanate is reacted with the intermediate of Formula (XXV) to give a protected urea of Formula (Xf). The benzoyl group is removed from the final molecule after combining Xf with the core molecule. In the cases that the isocyanate of Formula R¹⁻⁶NCO is not commercially available (and R¹⁻³ is H), it can conveniently be prepared by treatment of the amine of Formula R¹⁻⁶NH₂, wherein R¹⁻⁶ is aryl or heteroaryl, with phosgene, diphosgene or triphosgene in a suitable solvent such as ethyl acetate. When R¹⁻⁶ is alkyl or substituted alkyl, the preferred method is to treat the corresponding alkyl halide or diallyl sulfate with inorganic cyanates. These methods, as well as others, are well known to those skilled in the art and examples are described in S. R. Sandler and W. Karo “Organic Functional Group Preparations,” vol 12, 2^{nd} ed., p 364-375, 1983, Academic Press and references cited therein. In the cases that the carbamoyl chloride of Formula R¹⁻⁶ R¹⁻³NCOCl is not commercially available, it can conveniently be prepared by treatment of the amine of Formula R¹⁻⁶ R¹⁻³NH with phosgene, diphosgene or triphosgene in a suitable solvent such as dichloromethane at 0-4° C. Optionally, the N-benzyl protected amine of Formula R¹⁻⁶ R¹⁻³NCH₂(C₅H₆) can be reacted with triphosgene as described by M. G. Banwell, et al, J. Org. Chem. 2003, 68, 613-616.

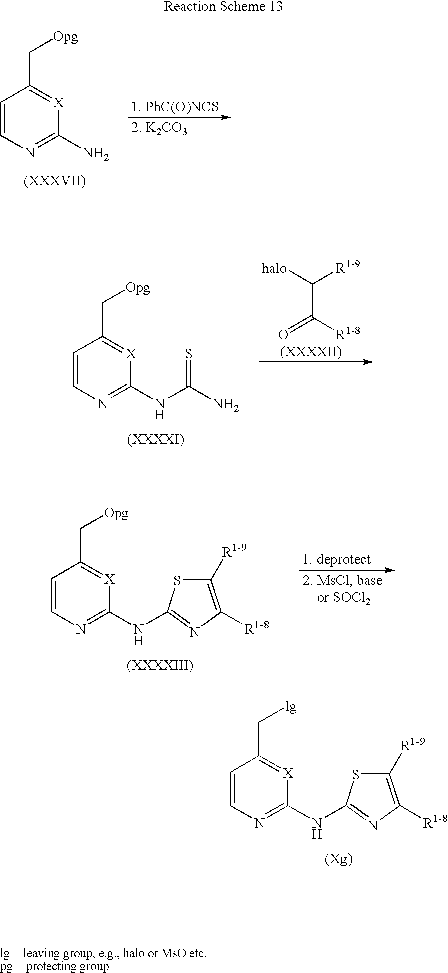

Compounds of Formula (Xg), which corresponds to Formula (X) where R³ is

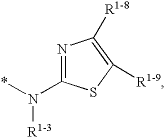

can be prepared by the route shown in Reaction Scheme 13. The intermediate of Formula (XXXVII), prepared as in Reaction Scheme 7, is allowed to react with benzoyl isothiocyanate, followed by a base such as potassium carbonate, to form the thiourea intermediate of Formula (XXXXI). This thiourea (XXXXI) is then allowed to react with a 2-haloketone of Formula (XXXXII) in the presence of a base, to form the thiazole intermediate of Formula (XXXXIII). Deprotection and conversion of the alcohol to a leaving group, e.g. mesylate, completes the synthesis of the intermediate of Formula (Xg).

A variety of compounds of Formula (I) can be prepared by elaboration of compounds, also of Formula (I), prepared by the above schemes. These elaboration methods are illustrated below in Reaction Schemes 14-17.

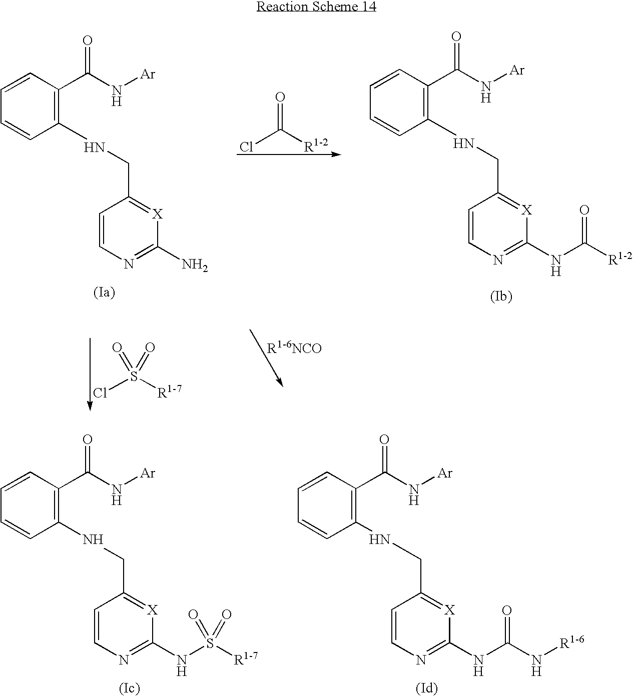

For example, the amino compound of Formula (Ia) can be converted to the amide compound of Formula (Ib), the sulfonamide of Formula (Ic) or the urea of Formula (Id) as shown in Reaction Scheme 14, by reaction with an acid chloride, sulfonyl chloride or isocyanate, respectively.

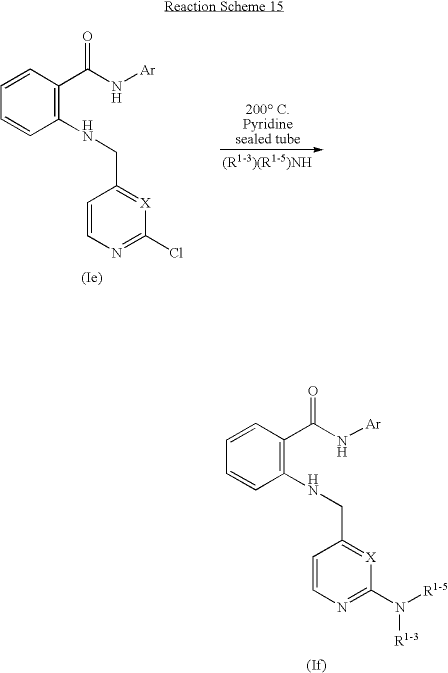

Additionally, the chloro compound of Formula (Ia) can be converted to the substituted amino compound of Formula (If) by reaction with an amine and a base such as pyridine in a sealed tube at elevated temperatures.

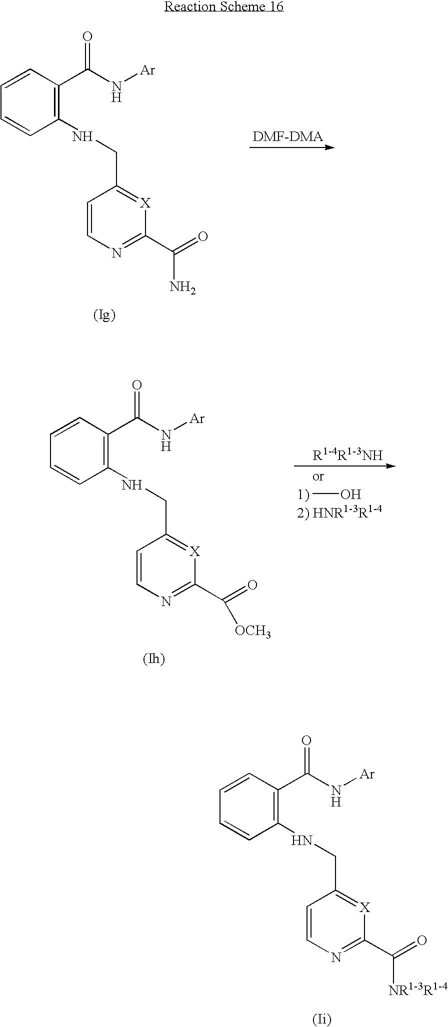

Esters of Formula (Ih) and substituted amides of Formula (II) may be prepared from the unsubstituted amide of Formula (Ig) by the sequence illustrated in Reaction Scheme 16. Reaction of the amide (Ig) with dimethylformamide-dimethylacetal in methanol provides the ester of Formula (Ih); reaction of the ester with a substituted amine gives the amide of Formula (II).

Generally, a desired salt of a compound of this invention can be prepared in situ during the final isolation and purification of a compound by means well known in the art. Or, a desired salt can be prepared by separately reacting the purified compound in its free base form with a suitable organic or inorganic acid and isolating the salt thus formed. These methods are conventional and would be readily apparent to one skilled in the art.

Additionally, sensitive or reactive groups on the compound of this invention may need to be protected and deprotected during any of the above methods. Protecting groups in general may be added and removed by conventional methods well known in the art (see, for example, T. W. Greene and P. G. M. Wuts, Protective Groups in Organic Synthesis; Wiley: New York, (1999).

By using the above illustrated general schemes and choosing the appropriate starting materials the compounds of the invention may be prepared. To further illustrate the invention, the following specific examples are provided, but are not meant to limit the scope of the invention in any way.

### ABBREVIATIONS AND ACRONYMS

When the following abbreviations are used throughout the disclosure, they have the following meanings:
ACN acetonitrile
AlMe₃ trimethylaluminum
anhyd anhydrous
Biotage® registered trademark of Biotage Corp. brand of MPLC
CDCl₃-d chloroform-d
CD₂Cl₂-d₂ methylene chloride-d₂
Celite® registered trademark of Celite Corp. brand of diatomaceous earth
CDCl₃ chloroform-d
d day(s)
DMF N,N-dimethyl formamide
DMSO-d₆ dimethylsulfoxide-d₆
EDAC 1-(3-Dimethylaminopropyl)-3-ethylcarbodiimide hydrochloride
EDCI 1-(3-Dimethylaminopropyl)-3-ethylcarbodiimide hydrochloride
EtOAc ethyl acetate
EtOH ethanol
equiv equivalent(s)
h hour(s)
¹H NMR proton nuclear magnetic resonance
Hex hexanes
HPLC high performance liquid chromatography
LCMS liquid chromatography/mass spectroscopy
MeOH methanol
MeOD-d₄ methanol-d₄
MgSO₄ magnesium sulfate
min minute(s)
MPLC medium pressure liquid chromatography
MS mass spectrometry
Pd/C palladium on carbon
R_{f} TLC retention factor
rt room temperature
RT retention time (HPLC or LCMS)
satd saturated
TEA triethylamine
THF tetrahydrofuran
TLC thin layer chromatography

### General Analytical Procedures

The structure of representative compounds of this invention were confirmed using the following procedures.

Electron impact mass spectra (EI-MS) were obtained with a Hewlett Packard 5989A mass spectrometer equipped with a Hewlett Packard 5890 Gas Chromatograph with a J & W DB-5 column (0.25 μM coating; 30 m×0.25 mm). The ion source is maintained at 250° C. and spectra were scanned from 50-800 amu at 2 sec per scan.

High pressure liquid chromatography-electrospray mass spectra (LC-MS) were obtained using either a:

(A) Hewlett-Packard 1100 HPLC equipped with a quaternary pump, a variable wavelength detector set at 254 nm, a YMC pro C-18 column (2×23 mm, 120 A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 minutes at a flowrate of 1.0 mL/min is used with an initial hold of 0.5 minutes and a final hold at 95% B of 0.5 minutes. Total run time is 6.5 minutes.
or
(B) Gilson HPLC system equipped with two Gilson 306 pumps, a Gilson 215 Autosampler, a Gilson diode array detector, a YMC Pro C-18 column (2×23 mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data is also acquired as an analog channel. The eluents were A: 2% acetonitrile in water with 0.02% TFA and B: 2% water in acetonitrile with 0.018% TFA. Gradient elution from 10% B to 90% over 3.5 minutes at a flowrate of 1.5 mL/min is used with an initial hold of 0.5 minutes and a final hold at 90% B of 0.5 minutes. Total run time is 4.8 minutes. An extra switching valve is used for column switching and regeneration.

Routine one-dimensional NMR spectroscopy is performed on 400 MHz Varian Mercury-plus spectrometers. The samples were dissolved in deuterated solvents obtained from Cambridge Isotope Labs, and transferred to 5 mm ID Wilmad NMR tubes. The spectra were acquired at 293 K. The chemical shifts were recorded on the ppm scale and were referenced to the appropriate solvent signals, such as 2.49 ppm for DMSO-d₆, 1.93 ppm for CD₃CN-d₃, 3.30 ppm for MeOD-d₄, 5.32 ppm for CD₂Cl₂-d₂ and 7.26 ppm for CDCl₃-d for ¹H spectra.

### DEFINITIONS

EPA Vial: Environmental Protection Agency Vial, 40 mL size with teflon septum cap. Sold by many vendors.

J-Kem Block: J-Kem Scientific, Inc. 6970 Olive BLVD, St. Louis, Mo. 63130. Reflux Reaction Block sold by J-Kem, customized to fit 40 mL EPA Vials, 9×7 array, 34.2 cm×30.5 cm×8 cm, 28.2 mm id hole size to accommodate EPA Vials. Block shakes on a typical orbital shaker such as one sold by J-Kem, model BTS 3000.

Mega: Centrifugal Vacuum Evaporator (speedvac) sold by Genevac, Inc, 707 Executive Blvd. Suite D, Valley Cottage, N.Y. 10989. Mega 1200 or Mega 980 floor model used equipped with Mega adapter plates to hold customized Gilson-type 207 racks, also sold by Genevac, using 16×100 mm test tubes.

MTP: Microtiter Plate. 2 mL deep-well plate used.

Tecan: Tecan US, P.O. Box 13953, Research Trianl Park, N.C. 27709. Tecan Genesis 200 used, 2 m deck size used with Genesis software, version 3.20 used. Customized, in-house written, Microsoft Visual Basic program was used to generate the Tecan Worldist for the fraction pooling operation.

Speedvac: HT8 Series II Centrifugal Vacuum Evaporator (speedvac) sold by Genevac, Inc, 707 Executive Blvd. Suite D, Valley Cottage, N.Y. 10989. The vials 24.6 mm (diameter)×54.75 mm (height) containing the pooled fractions in DMSO are dried using specially designed 8-position, MTP-footprint racks which fit into the Genevac holders (127 mm length×86.1 mm width×43.2 mm height).

### Starting Materials and Intermediates

Starting materials, i.e., compounds of formulae (V), (VI), (IX), (X), (XI), (XIII), (XIV), (XVI), (XVII), (XIX), (XXa), (XXb), (XXI) and (XXIII), used in the above Reaction Schemes 1-5, are either commercially available, or can be prepared by means well known in the art. Examples of such starting materials appear in Table 1 below.

TABLE 1
REFERENCE TABLE FOR SOURCES AND
PREPARATIVE METHODS OF STARTING MATERIALS

| Intermediate | Source |
|---|---|
| 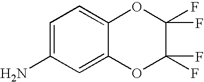 | Commercially availablefrom Lancaster. |
| 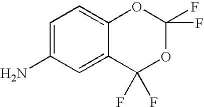 | Commercially availablefrom Lancaster. |
| 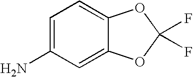 | Commercially availablefrom Lancaster. |
| 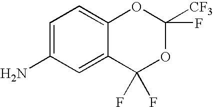 | Obtained from BayerChemicals, Germany.Reference: Alles, H. U.; Klauke,E. Fluorinated 1,3-benzodioxans. U.S. Pat. No.3,632,820, 1972. |
| 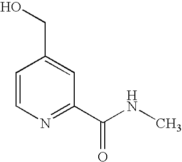 | Reference: Dumas, J. P.; Boyer,S. J.; Dixon, J. A.; Joe, T. K.;Kluender, H. C.; Lee, W.;Nagarathnam, D.; Sibley, R. N.;Su, Ning. Substituted Pyridinesand Pyridazines with AngiogenesisInhibiting Activity. U.S. Pat. No.6,689,883, 2004. |
| 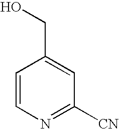 | Reference: Ple, P. A.; Green, T. P.;Hennequin, L.; Curwen, J.;Fennell, M.; Allen, J.; Lambert-van der Brempt, C.; Costello, G.Discovery of a New Class ofAnilinoquinazolines Inhibitors withHigh Affinity and Specificity forthe Tyrosine Kinase Domain ofc-Src. J. Med. Chem.2004, 47, 871-887. |
| 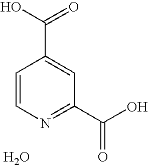 | Commercially available. |
| 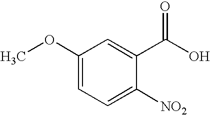 | Commercially available. |
| 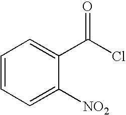 | Commercially available. |
| 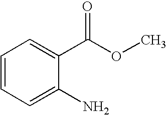 | Commercially available. |
| 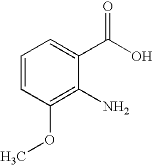 | Commercially available. |
| 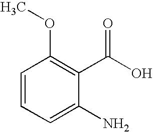 | Commercially available. |
| 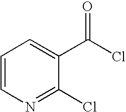 | Commercially available. |
| 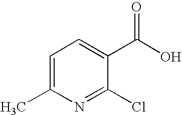 | Commercially available. |
| 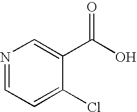 | Commercially available. |
| 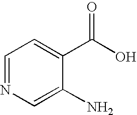 | Commercially available. |
| 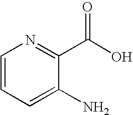 | Commercially available. |
| 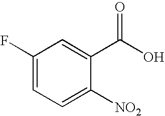 | Commercially available. |
| 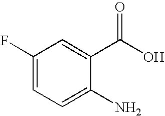 | Commercially available. |
| 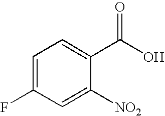 | Commercially available. |
| 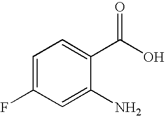 | Commercially available. |
| 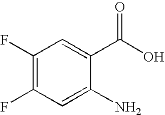 | Commercially available. |
| 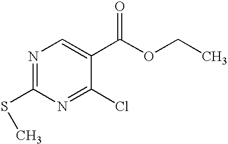 | Commercially available. |
| 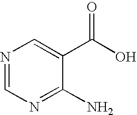 | Commercially available. |
| 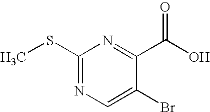 | Commercially available. |
| 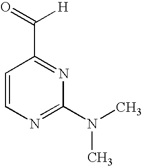 | Reference: Adams, J. L.; Boehm,J. C.; Kassis, S.; Gorycki,P. D.; Webb, E. F.; Hall, R.;Sorenson, M.;Lee, J. C.; Ayrton, A.;Griswold, D. E.; Gallagher, T. F.Pyrimidinylimidazole Inhibitorsof CSBP/P38 KinaseDemonstrating DecreasedInhibition ofHepatic Cytochrome P450Enzymes. Bioorg. Med. Chem.Lett. 1998, 8, 3111-3116. |
| 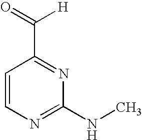 | Reference: Adams, J. L.; Boehm,J. C.; Kassis, S.; Gorycki,P. D.; Webb, E. F.; Hall, R.;Sorenson, M.;Lee, J. C.; Ayrton, A.;Griswold, D. E.; Gallagher, T. F.Pyrimidinylimidazole Inhibitorsof CSBP/P38 KinaseDemonstrating DecreasedInhibition ofHepatic Cytochrome P450Enzymes. Bioorg. Med. Chem.Lett. 1998, 8, 3111-3116. |
| 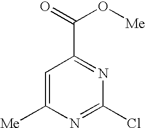 | Commercially Available |
| 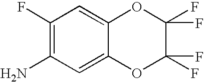 | Available throughcommercial sources |
| 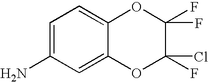 | Available throughcommercial sources |
| 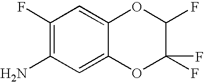 | Available throughcommercial sources |
| 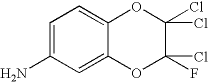 | Available throughcommercial sources |
| 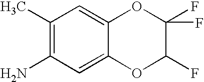 | Available throughcommercial sources |
| 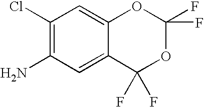 | Available throughcommercial sources |
| 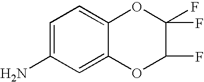 | Available throughcommercial sources |
| 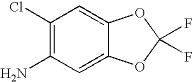 | Available throughcommercial sources |
| 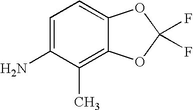 | Available throughcommercial sources |
| 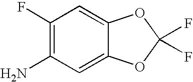 | Available throughcommercial sources |
| 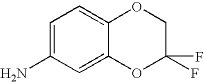 | Available throughcommercial sources |

### PREPARATION OF INTERMEDIATES

### Intermediate A: Preparation of {2-[(methylamino)carbonyl]pyridin-4-yl}methyl methanesulfonate

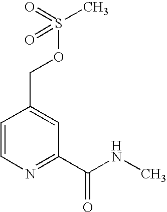

To a solution of 4-(hydroxymethyl)-N-methylpyridine-2-carboxamide (9.78 g, 58.9 mmol) in THF (250 mL) was added triethyl amine (12.3 mL, 88.3 mmol). The reaction was cooled to 0° C. and methanesulfonyl chloride (5.5 mL, 70.6 mmol) was added dropwise over 15 min. The reaction was allowed to slowly come to room temperature and then stirred an additional 3 h. The resulting solution was concentrated, re-dissolved in EtOAc (200 mL), transferred into a separatory funnel and extracted with EtOAc (2×200 mL). The combined organic layers were washed with cold satd. NaHCO₃ (2×200 mL). The organic layer was dried (MgSO₄), filtered and concentrated to afford 1.16 g of the above compound as a solid (4.75 mmol, yield 81%). ¹H NMR (CD₂Cl₂-d₂) δ 8.59 (d, J=4.88 Hz, 1H), 8.15 to 8.16 (m, 1H), 7.48 to 7.50 (m, 1H), 5.31 (s, 2H), 3.10 (s, 3H), 3.01 (d, J=5.08 Hz, 3H); LCMS: 245 [M+H]⁺, RT 1.24 min.

### Intermediate B: Preparation of (2-cyanopyridin-4-yl)methyl methanesulfonate

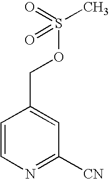

Same procedure as in Intermediate A except 4-(hydroxymethyl)pyridine-2-carbonitrile was used in place of 4-(hydroxymethyl)-N-methylpyridine-2-carboxamide.

¹H NMR (CDCl₃-d₃) 8.42 (d, J=5.02 Hz, 1H), 7.46 to 7.47 (m, 1H), 7.23 to 7.24 (m, 1H), 4.53 (s, 2H), 1.52 (s, 3H); LCMS: 363 [M+H]⁺.

### Intermediate C: Preparation of 4-(bromomethyl)-N-methylpyridine-2-carboxamide

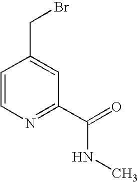

To a solution of 4-(hydroxymethyl)-N-methylpyridine-2-carboxamide (533 mg, 3.21 mmol) in THF (15 mL) was added triphenylphosphine (883.3 mg, 3.37 mmol) and carbon tetrabromide (1.1 g, 3.37 mmol). A white precipitate began to form quickly upon addition of the carbon tetrabromide. The mixture was allowed to stir at room temperature for 16 h. The mixture was then filtered to remove the precipitate and the filtrate was concentrated to oil. The crude product was purified via flash silica chromatography (40:60→60:40, EtOAc:Hex) to yield 345 mg (47%) of the product as a clear oil. The product did not appear to be stable as determined by rapid color change and was thus quickly used in the next step. LCMS: 229.1, 231.0 [M+H]⁺.

### Intermediate D: Preparation of dimethylpyridine-2,4-dicarboxylate

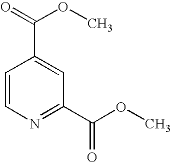

To a solution of 2,4-pyridinecarboxylic acid hydrate (505 mg, 2.73 mmol) in MeOH (5 mL) was added conc. H₂SO₄ (0.29 mL, 5.46 mmol). The solution was stirred until clear and trimethylorthoformate (1.2 mL, 10.9 mmol) was added to the reaction flask. The reaction was stirred at reflux for 16 h until complete. The resulting solution was concentrated in vacuo to afford 336 mg of the above compound as a solid (1.72 mmol, yield 63%). The crude material was used in further reactions without purification. ¹H NMR (CDCl₃-d) δ 8.90 (d, J=4.89 Hz, 1H), 8.65 to 8.66 (m, 1H), 8.03 to 8.04 (m, 1H), 4.05 (s, 3H), 4.01 (s, 3H); LCMS: 196 [M+H]⁺.

### Intermediate E: Preparation of ethyl 2-amino-3-methoxybenzoate

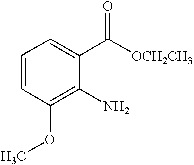

A mixture of 3-methoxyanthranilic acid (1 g, 6.0 mmol) in EtOH (10.0 mL) with TMSCl (3.8 mL, 30 mmol) was heated at reflux overnight. The reaction was evaporated to dryness, taken back up EtOAc, transferred to a separatory funnel and washed with sat. aq. NaHCO₃ (x2) and brine (x1). The organic layer was dried over anhy. Na₂SO₄ and evaporated to give compound that was pure enough to be used in the next step. ¹H NMR (CD₂Cl₂-d₂) δ 7.45 (d, J=8.0 Hz, 1H), 6.87 (d, J=8.0 Hz, 1H), 6.58 (dd, J=7.6, 8.0 Hz, 1H), 4.31 (q, J=7.0 Hz, 2H), 3.89 (s, 3H), 1.39 (t, J=6.5 Hz, 3H); LCMS: 196 [M+H]⁺, RT 2.88 min.

### Intermediate F: Preparation of ethyl 2-amino-6-methoxybenzoate

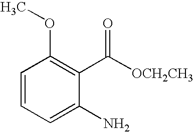

The experimental procedure was the same as described for Intermediate E.

### Intermediate G: Preparation of [2-(aminocarbonyl)pyridin-4-yl]methyl methanesulfonate

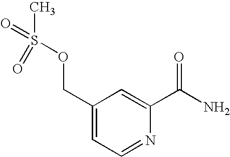

### Step 1: Preparation of ethyl 2-(aminocarbonyl)isonicotinate

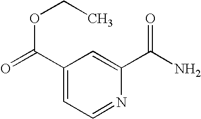

A solution of ethyl isonicotinate (25.2 mL, 165 mmol) in formamide (200 mL) was stirred with ice/methanol bath cooling as concentrated sulfuric acid (8.80 mL, 165 mmol) was added. Ferrous sulfate heptahydrate (69 g, 248 mmol) and hydrogen peroxide (25.6 mL of 30% in water) were added slowly over 25 min in alternating portions such that the temperature of the mixture was kept between 8-10.5° C. During this addition small pieces of dry ice were added to the bath to keep the reaction temperature in the desired range. After addition was complete, the ice bath was removed and the dark mixture was stirred for 2 h without cooling and then poured into a solution of trisodium citrate dihydrate (80.6 g) in water (700 mL) and then residues left in the reaction flask were washed out with a little methanol and water. The resulting mixture was rapidly stirred in a large flask as solid NaHCO₃ was added slowly, portion-wise, until the mixture was basic. Some saturated aqueous NaHCO₃ was added to make the mixture more basic and then it was vacuum filtered through Celite® and the solids were washed down with three 200 mL portions of dichloromethane. The phases of the filtrate were separated and the aqueous layer was extracted twice with dichloromethane. The combined extract was dried (Na₂SO₄) and evaporated in vacuo. The resulting solid residue was washed with ether/hexane (200 mL, 1:30) twice with warming and sonication followed by cooling and filtration to yield 13.9 g (44%) of pure title compound. The wash solutions, which contained some highly contaminated desired product, were discarded.

¹H NMR (300 MHz, DMSO-d₆) δ 8.83 (d, 1H), 8.39 (d, 1H, meta coupling), 8.24 (bs, 1H), 8.00 (d, 1H), 7.81 (bs, 1H), 4.39 (q, 2H) and 1.37 ppm (t, 3H); ES-MS m/z 195.0 [M+H]⁺, HPLC RT (min) 1.83.

### Step 2: Preparation of 4-(hydroxymethyl)pyridine-2-carboxamide

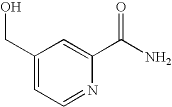

A slurry of ethyl 2-(aminocarbonyl)isonicotinate (5.00 g, 25.8 mmol) in absolute ethanol (150 mL) was stirred under nitrogen as sodium borohydride (2.92 g, 77.2 mmol) was added. After 22 h stirring at ambient temperature, the reaction was carefully quenched by addition of 17 mL of saturated aqueous ammonium chloride followed by stirring until the bubbling stopped and then evaporation in vacuo to leave a white solid residue. Saturated aqueous sodium chloride (80 mL) was added followed by five extractions with 200 mL portions of ethyl acetate. Combined extracts were dried (Na₂SO₄) and evaporated in vacuo to yield 3.85 g (98%) of pure title compound as a white solid.

¹H NMR (300 MHz, DMSO-d₆) δ 8.52 (d, 1H), 8.00 (s, 1H), 8.07 (bs, 1H), 7.46 (d, 1H), 7.60 (bs, 1H), 5.54 (t, 1H) and 4.60 ppm (d, 2H); ES-MS m/z 154.0 [M+H, weak signal]⁺, HPLC RT (min) 1.05.

### Step 3: Preparation of [2-(aminocarbonyl)pyridin-4-yl]methyl methanesulfonate

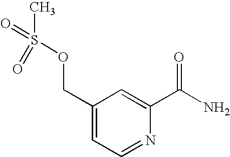

4-(hydroxymethyl)pyridine-2-carboxamide (1.00 g, 6.57 mmol) was dissolved in ethyl acetate (80 mL) and then cooled to 0° C. with stirring under nitrogen in an ice bath before triethylamine (1.37 mL, 9.86 mmol) was added, followed by methanesulfonyl chloride (0.66 mL, 8.54 mmol, added dropwise over 7 min). The ice bath was removed and the resulting suspension was stirred 2 h, and then the reaction mixture was poured into 60 mL water and stirred rapidly for 10 min. The phases were separated and the aqueous was extracted twice more with ethyl acetate. Each extract was washed with brine and the combined extracts were dried (Na₂SO₄) and evaporated in vacuo to yield 1.50 g (99%) of pure product as a fine white solid which turned pink on storage. Re-assay by NMR after such color change did not show significant decomposition.

¹H NMR (300 MHz, DMSO-d₆) δ 8.64 (d, 1H), 8.06 (s, 1H), 8.14 (bs, 1H), 7.6 (d, 1H), 7.70 (bs, 1H), 5.41 (s, 2H) and 3.33 ppm (s, overlaps with water in solvent).

### Intermediate I: Preparation of 2-{[4-(chloromethyl)pyridin-2-yl]amino}-2-oxoethyl acetate

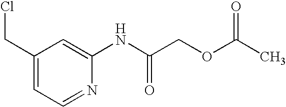

### Step 1: Preparation of 4-(chloromethyl)pyridin-2-amine

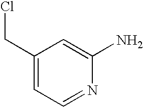

(2-Aminopyridin-4-yl)methanol (11.2 g, 90 mmol) was stirred in a flask with ice bath cooling as thionyl chloride (65.8 mL, 902 mmol) was slowly added. After about 10 mL was added, the temperature increased suddenly to about 50° C. and addition was halted as the mixture was broken up so that stirring could continue as the rest of the thionyl chloride was added. The cooling bath was then removed and the reaction was stirred for 2 h at ambient temperature before it was evaporated in vacuo and then toluene was added twice and evaporated each time in vacuo to yield the hydrochloride salt of the title compound. A suspension of this material in dichloromethane (150 mL) was stirred with saturated aqueous sodium bicarbonate (150 mL) for 1.5 h. The phases were separated and the organic extract was washed twice with water, once with brine and then dried (Na₂SO₄) and evaporated in vacuo to yield 10.71 g (83%) of pure title compound.

¹H NMR (300 MHz, DMSO-d₆) δ 7.87 (d, 1H), 6.48 (d, 1H), 6.45 (s, 1H), 6.04 (s, 2H) and 4.60 ppm (s, 2H); ES-MS m/z 143.2 [M+H]⁺, HPLC RT (min) 1.34.

### Step 2: Preparation of 2-{[4-(chloromethyl)pyridin-2-yl]amino}-2-oxoethyl acetate

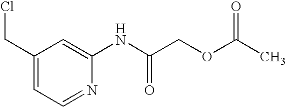

A suspension of 4-(chloromethyl)pyridin-2-amine (2.50 g, 10 mmol) and triethylamine (11.7 mL) in dichlioroethane (10 mL) was stirred under nitrogen with ice bath cooling as acetoxyacetyl chloride (1.86 mL, 17 mmol) was added slowly over 10 min. After 2 h stirring with cooling, TLC showed no starting material but three major product spots. The mixture was diluted with dichloromethane and washed with water and then brine. It was dried (Na₂SO₄) and evaporated in vacuo. The residue was purified by chromatography on silica gel using a gradient from 0-3% methanol in dichloromethane to yield 0.62 g (18%) of the correct and pure title compound.

¹H NMR (300 MHz, DMSO-d₆) δ 10.75 (s, 1H), 8.30 (d, 1H), 8.10 (bs, 1H), 7.17 (d, 1H), 4.79 (s, 2H), 4.71 (s, 2H) and 2.13 ppm (s, 3H); ES-MS m/z 243.1 [M+H]⁺, HPLC RT (min) 1.87.

### Intermediate J: Preparation of N-[4-(chloromethyl)pyridin-2-yl]acetamide

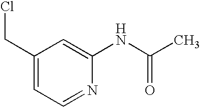

By using the methods described for preparation of Intermediate I and by substituting acetyl chloride instead of acetoxyacetyl chloride in step 2, Intermediate J was prepared from 2.30 g of 4-(chloromethyl)pyridin-2-amine and proportional amounts of other reagents. The yield of title compound was 2.0 g (67%) after silica gel chromatography. Even though examination of this material by NMR spectroscopy indicated that it was a mixture of the desired compound and the diacylated product N-acetyl-N-[4-(chloromethyl)pyridin-2-yl]acetamide (about 45:55), it was used as is in the next reaction and side products were separated by chromatography after the subsequent reaction.

¹H NMR (300 MHz, CD₂Cl₂) δ 8.33 (bs, 1H), 7.41 (d, 1H), 7.30 (s, 1H), 7.10 (d, 1H), 4.65 (s, 2H) and 2.20 ppm (s, 3H); ES-MS m/z 185.0 [M+H]⁺, HPLC RT (min) 1.16. Signals for the contaminating diacyl compound show at ¹H NMR (300 MHz, CD₂Cl₂) δ 8.56 (d, 1H), 8.18 (s, 1H), 78.24 (d, 1H), 4.75 (s, 2H) and 2.25 ppm (s, 6H); ES-MS m/z no significant M+H⁺ ion, HPLC RT (min) 0.97. Because of the closeness of the % content of the two compounds, it is possible that some of the NMR peak assignments have been switched between the desired material and the contaminant.

### Intermediate K: Preparation of N-[4-(chloromethyl)pyridin-2-yl]-2-methoxyacetamide

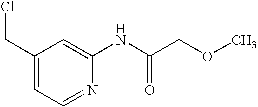

By using the methods described for preparation of Intermediate I and by substituting 2-methoxyacetyl chloride instead of acetoxyacetyl chloride in step 2, Intermediate K was prepared from 731 mg of 4-(chloromethyl)pyridin-2-amine and proportional amounts of other reagents. The yield of pure title compound was 397 mg (45%) after silica gel chromatography using a gradient from 0-40% ethyl acetate in hexane.

¹H NMR (300 MHz, CDCl₃) δ 9.00 (bs, 1H), 8.31 (d, 1H), 8.30 (s, 1H), 7.13 (d, 1H), 4.55 (s, 2H), 4.06 (s, 2H) and 3.51 ppm (s, 3H); ES-MS m/z 215.0 [M+H]⁺, HPLC RT (min) 0.71.

### Intermediate L: Preparation of N-[4-(chloromethyl)pyridin-2-yl]-2-(2-methoxyethoxy)acetamide

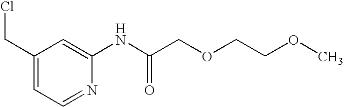

By using the methods described for preparation of Intermediate I and by substituting 2-(2-methoxyethoxy)acetyl chloride instead of acetoxyacetyl chloride in step 2, Intermediate L was prepared from 599 mg of 4-(chloromethyl)pyridin-2-amine and proportional amounts of other reagents. The yield of pure title compound was 314 mg (29%) after silica gel chromatography twice, first using a gradient from 2-3% methanol in dichloromethane, and then a second chromatography of the best fractions using a gradient from 0-40% ethyl acetate in hexane.

¹H NMR (300 MHz, CD₂Cl₂) δ 9.39 (bs, 1H), 8.30 (d, 1H), 8.29 (s, 1H), 7.13 (d, 1H), 4.59 (s, 2H), 4.14 (s, 2H), 3.76 (t, 2H), 3.60 (t, 2H) and 3.44 ppm (s, 3H); ES-MS m/z 259.1 [M+H]⁺, HPLC RT (min) 1.46.

### Intermediate M: Preparation of N-[4-(chloromethyl)pyridin-2-yl]-2-methoxypropanamide

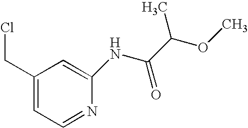

### Step 1: Preparation of 2-methoxypropanoic acid

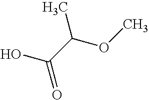

Sodium methoxide in methanol (25%, 16 mL) was added to a stirred solution of 2-bromopropionic acid (19.6 mmol) in methanol (5 mL) under nitrogen. The reaction was heated at 50° C. under nitrogen overnight. The reaction was then concentrated under vacuum. The residue was brought to pH 1 by the addition of 1 N aqueous HCl and this solution was then extracted with ethyl acetate three times (70 mL, 25 mL, 10 mL). The combined organic layer was dried (Na₂SO₄) and then concentrated under vacuum to yield the title compound as a colorless oil 2.04 g (99%) which was of sufficient purity to be used without purification. ¹H NMR (CD₃OD) δ3.67 (q, 1H), 3.33 (s, 3H), and 1.33 ppm (d, 3H).

### Step 2: Preparation of 2-methoxypropanoyl chloride

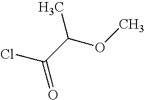

2-Methoxypropanoic acid (2.04 g, 19.2 mmol) was dissolved in dichloromethane (3 mL) which was stirred under nitrogen as a drop of dimethylformamide was added. Thionyl chloride was added dropwise into the reaction over 3 min and then the reaction was stirred at room temperature overnight. The reaction solution was concentrated in vacuo and the resulting pale yellow oil was placed under high vacuum to remove last traces of thionyl chloride. The yield of pure title compound was 303 mg (13%). ¹HNMR (CDCl₃) δ4.10 (q, 1H), 3.48 (s, 3H), and 1.56 ppm (d, 3H).

### Step 3: Preparation of N-[4-(chloromethyl)pyridin-2-yl]-2-methoxypropanamide

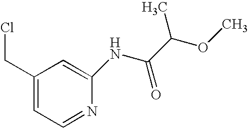

By using the methods described for preparation of Intermediate I (Step 2) and by substituting 2-methoxypropanoyl chloride instead of acetoxyacetyl chloride, Intermediate M was prepared from 352 mg of 4-(chloromethyl)pyridin-2-amine and proportional amounts of other reagents. The yield of pure title compound was 341 mg (60%) after silica gel chromatography using a gradient from 0-30% ethyl acetate in hexane.

¹H NMR (300 MHz, DMSO-d₆) δ 10.2 (bs, 1H), 8.30 (d, 1H), 8.17 (s, 1H), 7.16 (d, 1H), 4.77 (s, 2H), 4.00 (q, 1H), 3.26 (s, 3H), and 1.27 ppm (d, 3H).

### Intermediate N: Preparation of N-[4-(chloromethyl)pyridin-2-yl]-2-methoxy-2-methylpropanamide

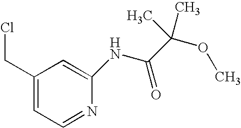

### Step 1: Preparation of 2-methoxy-2-methylpropanoic acid

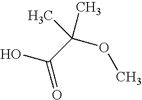

The procedure of Weizmann, Sulzbacher, and Bergmann as written in JACS 70,1153 (1948), which is hereby incorporated by reference, was used as follows: A solution of potassium hydroxide (8.96 g, 159.7 mmol) in 5 mL of water and 20 mL of methanol was stirred with ice bath cooling under nitrogen as 1,1,1-trichloro-2-methylpropan-2-ol (7.10 g, 40.0 mmol) was carefully added dropwise over ten min. Vigorous bubbling was observed as a white precipitate formed. The ice bath was removed after 15 min. The reaction was stirred at room temperature for 2 h then refluxed for 3 h. The reaction was cooled to room temperature and the solids were then removed by filtration and rinsed with methanol (350 mL). The filtrate was concentrated under vacuum to remove methanol and the remaining aqueous layer was brought to pH 0 by the addition of aqueous HCl then extracted with ethyl acetate (300 mL). The extract was dried (Na₂SO₄) and concentrated in vacuo to yield 4.11 g of crude product, which was purified by vacuum distillation to yield 2.28 g (48%) of the pure title compound as a colorless oil which was distilled at 105° C. (28 mm Hg). ¹HNMR (CDCl₃) δ 9.65 (s, 1H), 3.20 (s, 3H) and 1.32 ppm (s, 3H).

### Step 2: Preparation of 2-methoxy-2-methylpropanoyl chloride

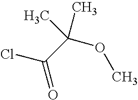

By following the procedure of Intermediate M (Step 2) but using 2-methoxy-2-methylpropanoic acid (6.99 g, 59.2 mmol) rather than 2-methoxypropanoic acid and proportional amounts of other reagents the title compound was synthesized. The crude product was distilled in vacuo to yield 2.671 g (33%) of pure compound, bp 44-48° C. (38 mmHg).

¹HNMR (CDCl₃) δ 3.33 (s, 3H) and 1.51 ppm (s, 6H).

### Step 3: Preparation of N-[4-(chloromethyl)pyridin-2-yl]-2-methoxy-2-methylpropanamide

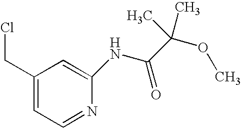

By using the methods described for preparation of Intermediate I (Step 2) and by substituting 2-methoxy-2-methylpropanoyl chloride instead of acetoxyacetyl chloride, Intermediate J was prepared from 1.04 g of 4-(chloromethyl)pyridin-2-amine and proportional amounts of other reagents. The yield of title compound was 1.23 g (69%) after silica gel chromatography using 30% ethyl acetate in hexane.

¹H NMR (300 MHz, DMSO-d₆) δ 9.41 (bs, 1H), 8.32 (d, 1H), 8.16 (s, 1H), 7.19 (d, 1H), 4.78 (s, 2H), 3.28 (s, 3H) and 1.36 ppm (s, 6H); ES-MS m/z 243.1 [M+H]⁺, HPLC RT (min) 2.12.

### Intermediate O: Preparation of N-[4-(chloromethyl)pyridin-2-yl]methanesulfonamide

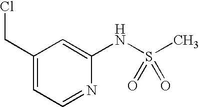

### Step 1: Preparation of N-[4-(chloromethyl)pyridin-2-yl]-N-(methylsulfonyl)methanesulfonamide

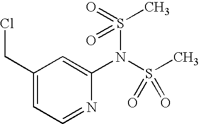

A solution of 4-(chloromethyl)pyridin-2-amine (500 mg, 3.51 mmol) and triethylamine (1.47 mL, 10.5 mmol) in ethyl acetate (4 mL) was stirred under nitrogen in a flask with ice bath cooling as methanesulfonyl chloride (0.81 mL, 10.5 mmol) was added dropwise. The reaction was then allowed to stir without cooling for 1 h before it was diluted with additional ethyl acetate, washed with water, dried (Na₂SO₄) and evaporated in vacuo. The resulting residue was purified by chromatography on silica gel using an ethyl acetate/hexane gradient to yield 860 mg (82%) of pure title compound.

¹H NMR (300 Hz, CD₂Cl₂) δ 8.56 (d, 1H), 7.50 (d, 1H), 7.41 (s, 1H), 4.66 (s, 2H), and 3.55 ppm (s, 6H); ES-MS m/z 299.0 [M+H]⁺, HPLC RT (min) 2.08.

### Step 2: Preparation of N-[4-(chloromethyl)pyridin-2-yl]methanesulfonamide

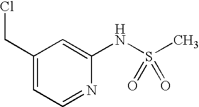

A suspension of N-[4-(chloromethyl)pyridin-2-yl]-N-(methylsulfonyl)-methanesulfonamide (700 mg, 2.34 mmol) in methanol (10 mL) and aqueous sodium hydroxide (1 N, 11.7 mL, 11.7 mmol) was stirred at ambient temperature as the starting material dissolved over 10 min. After another 10 min the reaction was adjusted to a pH between 3 and 6 by addition of aqueous hydrochloric acid (2 N) to precipitate the desired product as a white solid that was collected by filtration, washed with methanol and dried in vacuo. The yield of title compound was 250 mg (48%).

¹H NMR (300 MHz, DMSO-d₆) δ 10.93 (bs, 1H), 8.21 (d, 1H), 7.02 (m, 2H), 4.73 (s, 2H), and 3.23 ppm (s, 3H); ES-MS m/z 221.1 [M+H]⁺, HPLC RT (min) 1.45.

### Intermediate P: Preparation of N-[4-(chloromethyl)pyridin-2-yl]-N-ethylurea

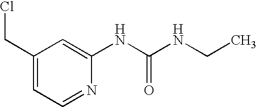

To 4-(chloromethyl)pyridin-2-amine (100 mg, 0.70 mmol) in 3 mL DMF was added ethyl isocyanate (59 mg, 0.84 mmol) and the resulting mixture was stirred under nitrogen for 16 h. The reaction was diluted with EtOAc (15 mL) and washed with H₂O three times, dried (Na₂SO₄) and evaporated in vacuo. The crude residue was purified by column chromatography on silica gel using 25% EtOAc in hexane to give 110 mg of N-[4-(chloromethyl)pyridin-2-yl]-N-ethylurea (73%).

¹H NMR (DMSO-d₆) δ 9.22 (s, 1H), 8.14-8.16 (m, 1H), 7.91-7.94 (m, 1H), 7.45 (d, J=0.8 Hz, 1H), 6.93-6.95 (m, 1H), 4.70 (s, 2H), 3.12-3.14 (m, 2H), 1.01-1.09 (m, 3H) ppm; LCMS: 214.1 [M+H]⁺, RT 0.47 min.

### Intermediate Q: Preparation of N-[4-(chloromethyl)pyridin-2-yl]-N′-phenylurea

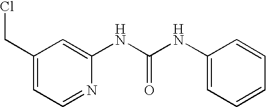

By using the methods described for preparation of Intermediate P and by substituting phenyl isocyanate instead of ethyl isocyanate, Intermediate Q was prepared. From 250 mg of 4-(chloromethyl)pyridin-2-amine and proportional amounts of other reagents the yield of title compound was 218 mg (47%) after silica gel chromatography using a gradient from 0-40% ethyl acetate in hexane. Though there was evidence of contamination with the starting material 4-(chloromethyl)pyridin-2-amine in the NMR spectrum, this material was used without further purification and side products were separated by chromatography after the next step.

¹H NMR (300 MHz, DMSO-d₆) δ 10.25 (bs, 1H), 9.50 (bs, 1H), 8.29 (d, 1H), 7.95 (s, 1H), 7.52 (d, 1H), 7.27-7.36 (m, 2H), 7.0-7.1 (m, 2H), and 4.79 ppm (s, 2H); LCMS: 262.2 [M+H]⁺, RT 2.65 min.

### Intermediate R: Preparation of N-[4-(chloromethyl)pyridin-2-yl]-N-methylurea

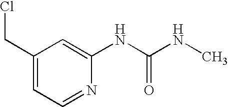

By using the methods described for preparation of Intermediate P and by substituting methyl isocyanate instead of ethyl isocyanate, Intermediate R was prepared. From 180 mg of 4-(chloromethyl)pyridin-2-amine and proportional amounts of other reagents the yield of pure title compound was 42 mg (17%) after silica gel chromatography using a gradient from 0-50% ethyl acetate in hexane followed by trituration of the residue with ether to remove a contaminant.

¹H NMR (DMSO-d₆) δ 9.31 (s, 1H), 8.16 (d, 1H), 7.92 (bm, 1H), 7.40 (s, 1H), 6.93 (d, 1H), 4.69 (s, 2H) and 2.70 ppm (d, 3H); LCMS: 200.1 [M+H]⁺, RT 1.17 min.

### Intermediate S: Preparation of 2,4-dichloro-6-(chloromethyl)pyrimidine

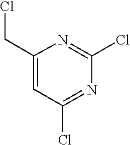

This product was prepared similarly to the 5-methyl substituted analog described in Biorg. Med. Chem. 2002, 10, 525, which is hereby incorporated by reference. A stirred suspension of 6-(chloromethyl)pyrimidine-2,4(1H, 3H)-dione (5.2 g, 32.6 mmol) in POCl₃ (9.1 mL, 97.9 mmol) was refluxed for 16 h under nitrogen. The mixture was cooled and evaporated to leave a dark colored oil. Ice water was slowly added and the product was extracted into dichloromethane. The organic layer was washed with brine, dried over MgSO₄, and concentrated under reduced pressure to give 2,4-dichloro-6-(chloromethyl)pyrimidine (5 g) as a yellow oil. Though this product could be used in the next step with out purification, another batch prepared in the same way was further purified by chromatography to show the following NMR.

¹H NMR (DMSO-d₆) δ 7.90 (s, 1H) and 4.78 ppm (s, 2H).

### Intermediate T: Preparation of 2-chloro-4-(chloromethyl)pyridine

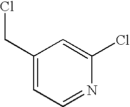

### Step 1: Preparation of (2-chloropyridin-4-yl)methanol

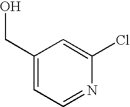

A sample of methyl 2-chloroisonicotinate (5.00 g, 29.14 nmol) was dissolved in 10 mL THF, treated with 10 drops of methanol, and cooled to 0° C. The solution was treated with lithium borohydride solution (21.86 mL of 1 M in THF, 43.71 mmol) and then allowed to warm to room temp. After 4 h the solution was cooled to 0° C. and quenched with 1 N HCl solution. The pH was adjusted to pH 10 with 1 N NaOH solution, and the reaction mixture was extracted with EtOAc. The organic extracts were washed with brine and concentrated in vacuo yielding 2.96 g (70.8%) of product.

¹H NMR (300 MHz, CD₃CN) δ 8.32 (d, 1H), 7.39 (s, 1H), 7.29 (d, 1H), 4.62 (s, 2H) and 3.53 ppm (bs, 1H).

### Step 2: Preparation of 2-chloro-4-(chloromethyl)pyridine

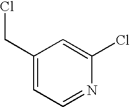

A sample of (2-chloropyridin-4-yl)methanol (110.0 mg, 0.77 mmol) was dissolved in anhydrous THF (1.5 mL), treated with N,N-diisopropylethylamine (0.29 mL, 1.69 mmol) and cooled to −78° C. Methanesulfonyl chloride was added (0.07 mL, 0.84 mmol), and the reaction mixture was allowed to slowly warm to room temperature overnight. The reaction mixture was then diluted with dichloromethane and washed with water. The organic layer was dried over Na₂SO₄ and concentrated in vacuo yielding the title compound (110.0 mg, 88.6%).

¹H NMR (300 MHz, CD₃CN) δ 8.40 (d, 1H), 7.49 (s, 1H), 7.39 (d, 1H) and 4.63 ppm (s, 2H); ES-MS m/z 183.2 [M+Na]⁺, HPLC RT (min) 2.30.

### Intermediate U: Preparation of 4-(chloromethyl)-N-(4-methyl-1,3-thiazol-2-yl)pyridin-2-amine

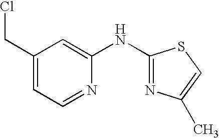

### Step 1: Preparation of 4-({[tert-butyl(dimethyl)silyl]oxy}methyl)pyridin-2-amine

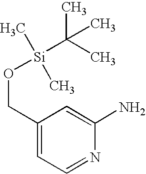

A solution of (2-aminopyridin-4-yl)methanol (5.0 g, 40 mmol), tert-butyldimethylsilyl chloride (6.07 g, 40 mmol), N-ethyl-N-isopropylpropan-2-amine (7.0 mL, 40 mmol) and N,N-dimethylpyridin-4-amine (0.49 g, 4 mmol) in dichloromethane (50 mL) was stirred 2 days at ambient temperature under nitrogen. The resulting reaction mixture was washed in sequence with aqueous sodium hydroxide (1 N), water and brine. It was then dried (Na₂SO₄) and concentrated in vacuo. The residue was chromatographed on silica gel using 50% ethyl acetate in hexane to yield pure title compound (5.47 g).

¹H NMR (300 MHz, CD₃CN) δ 7.75 (m, 1H), 6.39-6.48 (m, 2H), 4.70 (bs, 1H), 4. 50 (s, 2H), 0.83 (s, 9H) and 0.03 ppm (s, 6H); ES-MS m/z 239.3 [M+H]⁺, HPLC RT (min) 2.35.

### Step 2: Preparation of N-({[4-({[tert-butyl(dimethyl)silyl]oxy}methyl)pyridin-2-yl]amino}carbonothioyl)benzamide

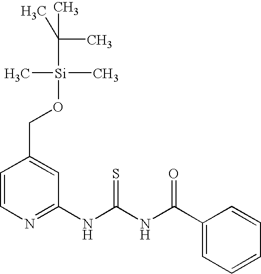

A solution of 4-({[tert-butyl(dimethyl)silyl]oxy}methyl)pyridin-2-amine (2.00 g, 8.39 mmol) and benzoyl isothiocyanate (1.51 g, 9.23 mmol) in toluene (20 mL) was heated to 85° C. for 12 h. The solvent was removed by evaporation in vacuo and the residue was purified by chromatography on silica gel using a gradient from 0-10% ethyl acetate in hexane to yield pure title compound as a yellow oil which solidified on standing (2.68 g, 79%).

¹H NMR (300 MHz, CD₃OD) δ 8.79 (bs, 1H), 8.18 (d, 1H), 7.83 (m, 2H), 7.50 (m, 1H), 7.40 (m, 2H), 7.04 (m, 1H), 4.68 (s, 2H), 0.82 (s, 9H), and 0.03 ppm (s, 6H); ES-MS m/z 402.0 [M+H]⁺, HPLC RT (min) 4.24.

### Step 3: Preparation of N-[4-({[tert-butyl(dimethyl)silyl]oxy}methyl)pyridin-2-yl]thiourea

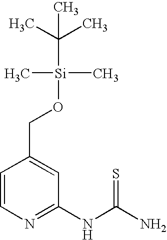

A solution of N-({[4-({[tert-butyl(dimethyl)silyl]oxy}methyl)pyridin-2-yl]amino}carbonothioyl)benzamide (1.00 g, 2.49 mmol) in absolute ethanol (15 mL) was stirred with potassium carbonate (0.344 g, 2.49 mmol) and heated to reflux under nitrogen for 16 h, after which the reaction mixture was filtered and the filtrate was evaporated under vacuum to give crude title compound (670 mg, >100%) as a white solid which was carried on to the next step without purification.

¹H NMR (300 MHz, DMSO-d₆) δ 10.55 (bs, 2H), 8.75 (bs, 1H), 8.05 (d, 1H), 7.10 (s, 1H), 6.83 (d, 1H), 4.60 (s, 2H), 0.83 (s, 9H) and 0.03 ppm (s, 6H); ES-MS m/z 298.2 [M+H]⁺, HPLC RT (min) 3.25.

### Step 4: Preparation of {2-[(4-methyl-1,3-thiazol-2-yl)amino]pyridin-4-yl}methanol

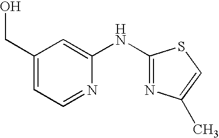

A solution of N-[4-({[tert-butyl(dimethyl)silyl]oxy}methyl)pyridin-2-yl]thiourea (crude material, 650 mg) and 1-chloroacetone (0.18 mL, 2.18 mmol) in ethanol (10 mL) was refluxed under nitrogen for 16 h and cooled. A white/pink solid was collected by filtration and washed with ethanol. The filtrate was evaporated in vacuo to yield a second white/pink solid. Comparison of the NMR of the two solids indicated that they were both the title compound and were pure enough (about 90%) to carry on to the next step without further purification (combined residue yield 516 mg, >100%).

¹H NMR (300 MHz, DMSO-d₆) δ 8.13 (d, 1H), 7.05 (s, 1H), 6.83 (d, 1H), 6.58 (s, 1H), 4.42 (s, 2H) and 2.18 ppm (s, 3H); ES-MS m/z 222.2 [M+H]⁺, HPLC RT (min) 1.41.

### Step 5: Preparation of 4-(chloromethyl)-N-(4-methyl-1,3-thiazol-2-yl)pyridin-2-amine

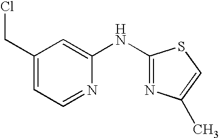

A mixture of {2-[(4-methyl-1,3-thiazol-2-yl)amino]pyridin-4-yl}methanol (200 mg, 0.9 mmol) and thionyl chloride (0.66 mL, 9.04 mmol) was stirred for 3 h and then evaporated in vacuo. The residue was dissolved in ethyl acetate and washed with saturated sodium bicarbonate. The aqueous layer was back extracted twice with ethyl acetate and then twice with a mixture of isopropanol, ethyl acetate and dichloromethane (1:8:1). The combined extracts were dried (Na₂SO₄) and concentrated in vacuo and the resulting residue was mixed with methanol, evaporated and then mixed with ethyl acetate and then evaporated again to yield the title compound as a light pink solid (200 mg, 92%) which was taken on to the next step as a crude solid.

¹H NMR (300 MHz, CD₂Cl₂) δ 8.30 (m, 1H), 6.98 (s, 1H), 6.90 (m, 1H), 6.50 (s, 1H), 4.55 (s, 2H) and 2.33 ppm (s, 3H); ES-MS m/z 240.2 [M+H]⁺, HPLC RT (min) 1.14.

### Intermediate V: Preparation of N-({[4-(chloromethyl)pyridin-2-yl]amino}carbonyl)benzamide

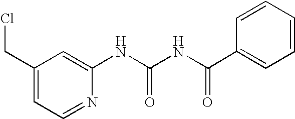

By using the methods described for preparation of Intermediate P and by substituting benzoyl isocyanate instead of ethyl isocyanate and using dichloromethane rather than DMF as solvent, Intermediate R was prepared. The product, which separated from the reaction mixture as a solid, was collected by filtration and washed with dichloromethane.

¹H NMR (DMSO-d₆) δ 11.01 (s, 1H), 10.98 (bs, 1H), 8.06 (d, 1H), 7.82 (s, 1H), 7.73 (d, 2H), 7.37 (t, 1H), 7.25 (t, 2H), 6.90 (d, 1H), and 4.52 (s, 2H).

Intermediate W: Preparation of 2-amino-N-(2,2-difluoro-1,3-benzodioxol-5-yl)benzamide

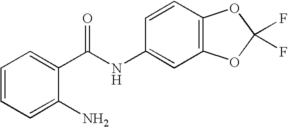

### Step 1: Preparation of N-(2,2-difluoro-1,3-benzodioxol-5-yl)-2-nitrobenzamide

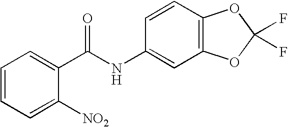

To a solution of 2,2-difluoro-1,3-benzodioxol-5-amine (19.08 g, 110.21 mmol) in CH₂Cl₂ (250 mL) was added triethylamine (38 mL, 275.54 mmol). 2-Nitrobenzoyl chloride (17 mL, 113.52) was dissolved in CH₂Cl₂ (70 mL) and added dropwise to the 2,2-difluoro-1,3-benzodioxol-5-amine solution over 1 h. The reaction was allowed to stir for 3 h. The solids formed were filtered off and the organic layer was washed with water, dried with Na₂SO₄, and evaporated. The crude material was used in the next step with out further purification LCMS: 322.9 [M+H]⁺, RT 3.11 min.

### Step 2: Preparation of 2-amino-N-(2,2-difluoro-1,3-benzodioxol-5-yl)benzamide

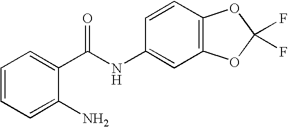

To a slurry of Pd/C (10%, 1.5 g) in EtOH (25 mL) under a static N₂ atmosphere was added a solution of N-(2,2-difluoro-1,3-benzodioxol-5-yl)-2-nitrobenzamide (30 g, 93.10 mmol) in EtOH (900 mL). The resulting mixture was stirred under a H₂ atmosphere (1 atm.) for 17 h, then filtered through a pad of Celite®. The filtrate was concentrated under reduced pressure, treated with EtOH and concentrated to afford a yellow solid. The compound was stirred in hexanes and the product precipitated out. The white solid was filtered and dried (25 g, 93%): ¹H NMR (DMSO-d₆) 6.32 (br s, 2H), 6.57 (t, 1H), 6.72 (d, 1H), 7.18 (m, 1H), 7.33 (d, 1H), 7.40 (d, 1H), 7.58 (d, 1H), 7.81 (s, 1H) 10.14 (s, 1H); LCMS: 293 [M+H]⁺, RT 2.84 min.

### Intermediate X: Preparation of (2-chloro-6-methylpyrimidin-4-yl)methyl methanesulfonate

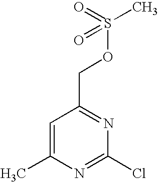

### Step 1: Preparation of (2-chloro-6-methylpyrimidin-4-yl)methanol

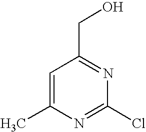

To a 0° C. solution of methyl 2-chloro-6-methylpyrimidine-4-carboxylate (750 mg, 4.02 mmol) in MeOH (4.5 mL)/water (0.4 mL) was added sodium borohydride (190 mg, 5.02 mmol) portionwise. The reaction was allowed to slowly come to room temperature and then stirred an additional 16 h. The resulting solution was concentrated, re-dissolved in EtOAc, transferred into a separatory funnel and extracted with water. The combined organic layers were washed with brine. The organic layer was dried (MgSO₄), filtered and concentrated to afford 484 mg of the crude 4-(hydroxymethyl)-2-chloro-6-methylpyrimidine as a brown solid (yield 76%). LCMS: 159 [M+H]⁺, RT 1.02 min.

### Step 2: Preparation of (2-chloro-6-methylpyrimidin-4-yl)methyl methanesulfonate

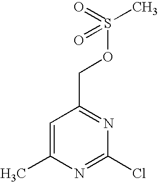

To a solution of the crude 4-(hydroxymethyl)-2-chloro-6-methylpyrimidine (484 mg, 3.05 mmol) in THF (10 mL) was added triethyl amine (0.55 mL, 3.97 mmol). The reaction was cooled to 0° C. and methanesulfonyl chloride (0.28 mL, 3.66 mmol) was added dropwise. The reaction was allowed to slowly come to room temperature and then stirred an additional 16 h. The resulting solution was concentrated, re-dissolved in EtOAc, transferred into a separatory funnel and extracted with EtOAc. The combined organic layers were washed with cold sat'd. NaHCO₃. The organic layer was dried (MgSO₄), filtered and concentrated to afford 750 mg of the above compound as a crude brown oil. LCMS: 237 [M+H]⁺, RT 1.50 min.

### PREPARATIVE EXAMPLES OF THE COMPOUNDS OF THE INVENTION

### Example 1

### 5-methoxy-2-[(pyridin-4-ylmethyl)amino]-N-(2,2,3,3-tetrafluoro-2,3-dihydro-1,4-benzodioxin-6-yl)benzamide

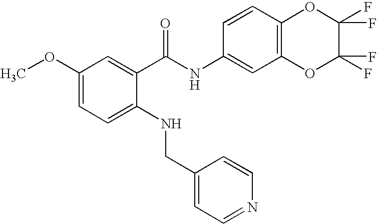

### Step 1: Preparation of 5-methoxy-2-nitro-N-(2,2,3,3-tetrafluoro-2,3-dihydro-1,4-benzodioxin-6-yl)benzamide

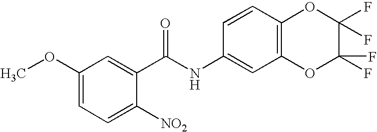

To a solution of 5-methoxy-2-nitrobenzoic acid (2.10 g, 10.7 mmol) in CH₂Cl₂ (0.2 mL) was added DMF (0.2 mL) followed by oxalyl chloride (1.86 mL, 21.3 mmol). After the cessation of gas evolution, the resulting mixture was stirred at rt for 1 h, then concentrated under reduced pressure. The resulting yellow solid was dissolved in THF (10 mL) and TEA (2.2 mL, 16.0 mmol), cooled to 0° C., and treated with a solution of 2,2,3,3-tetrafluoro-2,3-dihydro-1,4-benzodioxin-6-amine (2.50 g, 11.2 mmol) in THF (40 mL) dropwise. The resulting thick slurry was allowed to warm to room temp and was stirred for 1 h. The resulting mixture was treated with CH₂Cl₂ and sequentially washed with a 1 N HCl solution (500 mL) and a saturated NaCl solution (500 mL). The organic layer was dried (Na₂SO₄) and concentrated under reduced pressure to give 5-methoxy-2-nitro-N-(2,2,3,3-tetrafluoro-2,3-dihydro-1,4-benzodioxin-6-yl)benzamide as a yellow foam (4.4 g, 100%): TLC (30% EtOAc/hexane) R_{f} 0.27; LCMS: 403 [M+H]⁺, RT 3.43 min.

### Step 2: Preparation of 2-amino-5-methoxy-N-(2,2,3,3-tetrafluoro-2,3-dihydro-1,4-benzodioxin-6-yl)benzamide

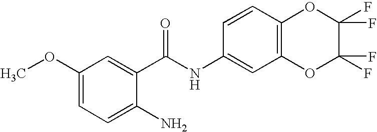

To a slurry of Pd/C (5%, 0.4 g) in MeOH (10 mL) under a static N₂ atmosphere was added a solution of 5-methoxy-2-nitro-N-(2,2,3,3-tetrafluoro-2,3-dihydro-1,4-benzodioxin-6-yl)benzamide (4.30 g, 10.7 mmol) in MeOH (190 mL). The resulting mixture was stirred under a H₂ atmosphere (1 atm.) for 17 h, then filtered through a pad of Celite®. The filtrate was concentrated under reduced pressure, treated with CH₂Cl₂ and concentrated again to afford a yellow solid. Further purification by MPLC (Biotage®, Flash 20M column, 20% EtOAc/hexane) afforded 2-amino-5-methoxy-N-(2,2,3,3-tetrafluoro-2,3-dihydro-1,4-benzodioxin-6-yl)benzamide as a white solid (2.08 g, 52%): TLC (20% EtOAc/hexane) R_{f} 0.31; ¹H NMR (DMSO-d₆) δ3.72 (s, 3H), 5.91 (br s, 2H), 6.73 (d, J=8.9 Hz, 1H), 6.94 (dd, J=8.9, 2.8 Hz, 1H), 7.16 (d, J=2.9 Hz, 1H), 7.45 (d, J=9.2 Hz, 1H), 7.58 (dd, J=9.0, 2.3 Hz, 1H), 7.88 (d, J=2.3 Hz, 1H), 10.26 (br s, 1H); LCMS: 373 [M+H]⁺, RT 3.05 min.

### Step 3: Preparation of 5-methoxy-2-[(pyridin-4-ylmethyl)amino]-N-(2,2,3,3-tetrafluoro-2,3-dihydro-1,4-benzodioxin-6-yl)benzamide

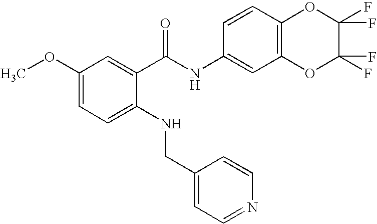

A solution of 2-amino-5-methoxy-N-(2,2,3,3-tetrafluoro-2,3-dihydro-1,4-benzodioxin-6-yl)benzamide (0.20 g, 0.54 mmol) in MeOH (2.0 mL) was sequentially treated with acetic acid (0.2 mL, 0.35 mmol) and 4-pyridinecarboxaldehyde (0.07 g, 0.64 mmol), and the resulting mixture was allowed to stir for 16 h. Sodium cyanoborohydride (0.11 g, 1.72 mmol) was then added. After gas evolution had subsided, the reaction was allowed to stir at room temp. for 72 h. The resulting mixture was treated with a saturated NaHCO₃ solution (50 mL) and extracted with CH₂Cl₂ (50 mL). The organic layer was concentrated under reduced pressure. The residue was purified by MPLC (Biotage®, Flash 12M column, 50% EtOAc/hexane) followed by repurification (Biotage®, Flash 12M column, 30% EtOAc/hexane) to afford 5-methoxy-2-[(pyridin-4-ylmethyl)amino]-N-(2,2,3,3-tetrafluoro-2,3-dihydro-1,4-benzodioxin-6-yl)benzamide (0.41 g, 16%) as a white solid: TLC (70% EtOAc/hexane) R_{f} 0.40; ¹H NMR (DMSO-d₆) δ3.68 (s, 3H), 6.41 (d, J=3.6 Hz, 1H), 6.78 (d, J=8.8 Hz, 1H), 6.89 (dd, J=8.8, 3.0 Hz, 1H), 7.21 (d, J=3.0 Hz, 1H), 7.30 (dd, J=9.0, 2.5 Hz, 1H), 7.34-7.36 (m, 2H), 7.47-7.55 (m, 4H), 8.50-8.52 (m, 2H); LCMS: 434 [M+H]⁺, RT 2.50 min.

### Example 2

### 2-[(pyridin-4-ylmethyl)amino]-N-(2,2,4,4-tetrafluoro-4H-1,3-benzodioxin-6-yl)benzamide

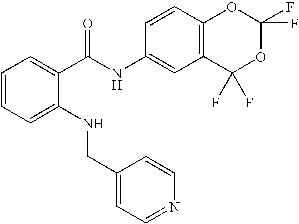

This compound was synthesized using the same synthetic route as Example 1 except that in Step 1, the compound 2-nitrobenzoyl chloride was used in place of forming 5-methoxy-2-nitrobenzoyl chloride in situ and 2,2,4,4-tetrafluoro-4H-1,3-benzodioxin-6-amine was used in place of 2,2,3,3-tetrafluoro-2,3-dihydro-1,4-benzodioxin-6-amine.

¹H NMR (DMSO-d₆) δ 10.54 (s, 1H), 8.73 (d, J=5.5 Hz, 2H), 8.40 (d, J=2.9 Hz, 1H), 7.99, (dd, J=2.4, 4.8 Hz, 2H), 7.78 (d, J=6 Hz, 2H), 7.23 (dd, J=1.8, 4.3 Hz, 1H), 7.50 (d, J=8.4 Hz, 1H), 7.24 (t, J=7.2, 1H), 6.68 (t, J=8.4 Hz, 1H), 6.49 (d, J=8.4 Hz, 1H), 4.69 (s, 2H); LCMS: 431 [M+H]⁺, RT 3.11 min.

### Example 3

### N-methyl-4-{[(2-{[(2,2,3,3-tetrafluoro-2,3-dihydro-1,4-benzodioxin-6-yl)amino]carbonyl}phenyl)amino]methyl}pyridine-2-carboxamide

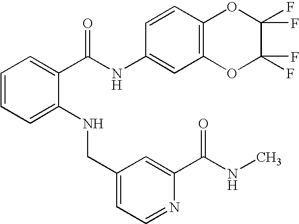

### Step 1: Preparation of 2-nitro-N-(2,2,3,3-tetrafluoro-2,3-dihydro-1,4-benzodioxin-6-yl)benzamide

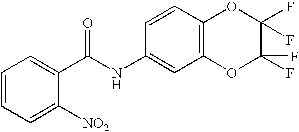

To a solution of 2,2,3,3-tetrafluoro-2,3-dihydro-1,4-benzodioxin-6-amine (2.53 g, 11 mmol) and TEA (1.65 mL, 12 mmol) in THF (50 mL) at 0° C. was added 2-nitrobenzoyl chloride (2.0 g, 11 mmol) dropwise. The resulting thick slurry was allowed to warm to rt and was stirred for 1 h. The resulting mixture was treated with CH₂Cl₂ and washed with a 1 N HCl solution (500 mL). The organic layer was dried (Na₂SO₄) and concentrated under reduced pressure to 2-nitro-N-(2,2,3,3-tetrafluoro-2,3-dihydro-1,4-benzodioxin-6-yl)benzamide as a beige solid (4.0 g, 100%): TLC (30% EtOAc/hexane) R_{f} 0.24; ¹H NMR (DMSO-d₆) δ7.49 (d, J=1.1 Hz, 2H), 7.76-7.89 (m, 4H), 8.17 (dd, J=8.2, 0.8 Hz, 1H), 11.00 (br s, 1H); LCMS: 373 [M+H]⁺, RT 3.43 min.

### Step 2: Preparation of 2-amino-N-(2,2,3,3-tetrafluoro-2,3-dihydro-1,4-benzodioxin-6-yl)benzamide

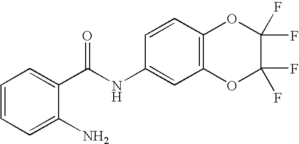

This compound was prepared using the same procedure as in Step 2 of Example 1 but using the product from step 1 as starting material.

¹H NMR (DMSO-d₆) δ 6.35 (br s, 2H), 6.58 (app td. J=7.5, 0.9, 1H), 6.74 (dd, J=8.0, 0.8 Hz, 1H), 7.20 (app td, J=7.2, 1.3 Hz, 1H), 7.23 (d, J=1.2 Hz, 1H), 7.42-7.60 (m, 2H), 7.88 (d, J=2.4 Hz, 1H, 10.25 (br s, 1H); LCMS: 343 [M+H]⁺, RT 3.48 min.

### Step 3: Preparation of N-methyl-4-{[(2-{[(2,2,3,3-tetrafluoro-2,3-dihydro-1,4-benzodioxin-6-yl)amino]carbonyl}phenyl)amino]methyl}pyridine-2-carboxamide

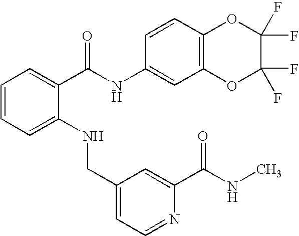

To a solution of Intermediate C (0.15 g, 0.65 mmol) in DMF (5 mL) was sequentially added 2-amino-N-(2,2,3,3-tetrafluoro-2,3-dihydro-1,4-benzodioxin-6-yl)benzamide (0.19 g, 0.55 mmol), K₂CO₃ (0.15 g, 1.09 mmol), and NaI (0.16 g, 1.09 mmol). The resulting burgundy-colored mixture was heated at 60° C. for 2 d, then cooled to rt and diluted with water (100 mL). The resulting mixture was extracted with CHCl₃ (4×100 mL). The combined organic layers were dried (Na₂SO₄) and concentrated under reduced pressure. The residue was purified by MPLC (Biotage®®, Flash 12M column, 30% EtOAc/hexane) to afford N-methyl-4-{[(2-{[(2,2,3,3-tetrafluoro-2,3-dihydro-1,4-benzodioxin-6-yl)amino]carbonyl}phenyl)amino]methyl}pyridine-2-carboxamide (0.04 g, 15%) as a pale yellow solid: TLC (40% EtOAc/hexane) R_{f} 0.23; ¹H NMR (DMSO-d₆) δ2.78 (d, J=5.0 Hz, 3H), 4.57 (d, J=6.0 Hz, 2H), 6.52 (dd, J=8.5, 0.8 Hz, 1H), 6.66 (app td, J=7.7, 1.0 Hz, 1H), 7.24 (app td, J=7.7, 1.6 Hz, 1H), 7.47 (d, J=9.2 Hz, 1H), 7.52 (dd, J=5.0, 1.8 Hz, 1H), 7.61 (dd, J=9.1, 2.5 Hz, 1H), 7.68 (dd, J=8.0, 1.7 Hz, 1H), 7.93 (d, J=2.3 Hz, 1H), 7.95-7.98 (m, 2H), 8.54 (dd, J=5.0, 0.7 Hz, 1H), 8.74 (br q, J=4.7 Hz, 1H), 10.45 (br s, 1H); LCMS: 491 [M+H]⁺, RT 3.59 min.

### Example 4

### N-methyl-4-{[(2-{[(2,2,4,4-tetrafluoro-4H-1,3-benzodioxin-6-yl)amino]carbonyl}phenyl)amino]methyl}pyridine-2-carboxamide

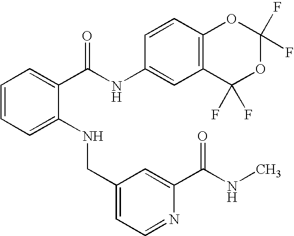

This compound was synthesized using the same synthetic route as Example 3 except that in Step 1,2,2,4,4-tetrafluoro-4H-1,3-benzodioxin-6-amine was used in place of 2,2,3,3-tetrafluoro-2,3-dihydro-1,4-benzodioxin-6-amine.

TLC (40% EtOAc/hexane) R_{f} 0.23; ¹H NMR (DMSO-d₆) δ 2.78 (d, J=4.8 Hz, 3H), 4.59 (d, J=6.0 Hz, 2H), 6.51 (dd, J=8.1, 0.7 Hz, 1H), 6.66 (app td, J=7.4, 1.1 Hz, 1H), 7.24 (app td, J=7.7, 1.6 Hz, 1H), 7.49-7.53 (m, 2H), 7.21 (dd, J=7.9, 1.6 Hz, 1H), 7.98-8.03 (m, 3H), 8.39 (d, J=2.4 Hz, 1H), 8.53 (dd, J=4.9, 0.8 Hz, 1H), 8.74 (br q, J=4.9 Hz, 1H), 10.54 (br s, 1H); LCMS: 491 [M+H]⁺, RT 3.56 min.

### Example 5

### N-(2,2-difluoro-1,3-benzodioxol-5-yl)-2-[(pyridin-4-ylmethyl)amino]benzamide

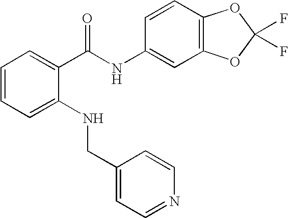

### Step 1: Preparation of methyl 2-[(pyridin-4-ylmethyl)amino]benzoate

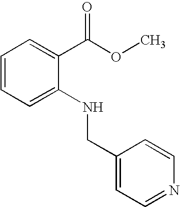

A solution of methyl anthranylate (10.0 g, 66.2 mmol), 4-pyridinecarboxaldehyde (8.50 g, 79.4 mmol), and acetic acid (5.00 mL) in MeOH (500 mL) was stirred at rt for 3 d. Sodium cyanoborohydride (13.3 g, 212 mmol) was then added in small portions. After gas evolution had subsided, the reaction was allowed to stir at room temp. for 2 d. The resulting mixture was treated with a saturated NaHCO₃ solution (500 mL) and extracted with EtOAc (500 mL). The organic layer was concentrated under reduced pressure. The residue was purified by MPLC (Biotage®, Flash 75M column, 40% EtOAc/hexane) to afford methyl 2-[(pyridin-4-ylmethyl)amino]benzoate (8.5 g, 53%) as a white solid: TLC (50% EtOAc/hexane) R_{f} 0.31; ¹H NMR (DMSO-d₆) δ 3.82 (s, 3H), 4.54 (d, J=6.1 Hz, 2H), 6.56 (br d, J=5.5 Hz, 1H), 5.59 (app td, J=7.7, 1.4 Hz, 1H), 7.26-7.32 (m, 1H), 7.31 (d, J=6.2 Hz, 2H), 7.81 (dd, J=8.0, 1.6 Hz, 1H), 8.20 (br t, J=6.2 Hz, 1H), 8.49 (d, J=6.0 Hz, 2H); LCMS: 243 [M+H]⁺, RT 1.71 min.

### Step 2: Preparation of N-(2,2-difluoro-1,3-benzodioxol-5-yl)-2-[(pyridin-4-ylmethyl)amino]benzamide

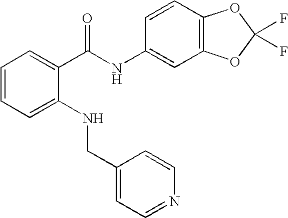

A solution of 2,2-difluoro-1,3-benzodioxol-5-amine (0.21 g, 1.24 mmol) in toluene (3.0 mL) at 0° C. was with AlMe₃ (2 M in heptane, 0.62 mL, 1.24 mmol). The resulting mixture was stirred at 0° C. for 1 h, followed by addition of methyl 2-[(4-pyridylmethyl)amino]benzoate (0.10 g, 0.41 mmol). The resulting mixture was stirred at 80° C. for 5 d, cooled to room temp., and treated with a saturated NaHCO₃ solution (100 mL). The resulting mixture was extracted with CH₂Cl₂ (3×100 mL). The combined organic layers were dried (Na₂SO₄) and concentrated reduced pressure. The residue was purified by MPLC (Biotage®, Flash 12M column, 30% EtOAc/hexane) to give N-(2,2-difluoro-1,3-benzodioxol-5-yl)-2-[(pyridin-4-ylmethyl)amino]benzamide (0.037 g, 23%) as a beige solid: TLC (50% EtOAc/hexane) R_{f} 0.30; ¹H NMR (DMSO-d₆) δ 4.48 (d, J=6.4 Hz, 2H), 6.54 (dd, J=8.5, 0.8 Hz, 1H), 6.65 (app td, J=7.5, 1.0 Hz, 1H), 7.24 (app td, J=7.8, 1.4 Hz, 1H), 7.32 (d, J=6.0 Hz, 2H), 7.38 (dd, J=9.1, 0.4 Hz, 1H), 7.45 (dd, J=8.8, 2.0 Hz, 1H), 7.86 (d, J=1.7 Hz, 1H), 7.89 (br t, J=6.5 Hz, 1H), 8.48 (d, J=6.05 Hz, 2H), 10.34 (br s, 1H); LCMS: 384 [M+H]⁺, RT 2.90 min.

### Example 6

### 2-[(pyridin-4-ylmethyl)amino]-N-(2,2,3,3-tetrafluoro-2,3-dihydro-1,4-benzodioxin-6-yl)benzamide

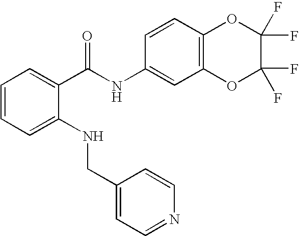

This compound was synthesized using the same synthetic route as Example 5 except that in Step 2,2,2,3,3-tetrafluoro-2,3-dihydro-1,4-benzodioxin-6-amine was used in place of 2,2-difluoro-1,3-benzodioxol-5-amine.

TLC (50% EtOAc/hexane) R_{f} 0.27; ¹H NMR (DMSO-d₆) δ 4.47 (d, J=6.1 Hz, 2H), 6.53 (d, J=8.4 Hz, 1H), 6.64 (app td, J=7.4, 0.9 Hz, 1H), 7.24 (app td, J=7.7, 1.5 Hz, 1H), 7.30 (d, J=5.7 Hz, 2H), 7.45 (d, J=9.0 Hz, 1H), 7.59 (dd, J=9.1, 2.3 Hz, 1H), 7.66 (dd, J=7.9, 1.4 Hz, 1H), 7.86 (br t, J=6.2 Hz, 1H), 7.90 (d, J=2.3 Hz, 1H), 8.47 (d, J=5.9 Hz, 2H), 10.41 (br s, 1H); LCMS: 434 [M+H]⁺, RT 3.15 min.

### Example 7

### 3-methoxy-2-[(pyridin-4-ylmethyl)amino]-N-(2,2,3,3-tetrafluoro-2,3-dihydro-1,4-benzodioxin-6-yl)benzamide

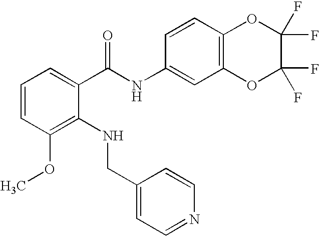

This compound was synthesized using the same synthetic route as Example 5 except that in Step 1, Intermediate E was used in place of methyl anthranylate, and in Step 2,2,2,3,3-tetrafluoro-2,3-dihydro-1,4-benzodioxin-6-amine was used in place of 2,2-difluoro-1,3-benzodioxol-5-amine.

¹H NMR (CD₂Cl₂-d₂) δ 10.7 (s, 1H), 8.75 (m, 2H), 7.84 (s, 1H), 7.78 (d, J=4.0 Hz, 1H), 7.29-6.93 (m, 7H), 4.21 (s, 2H), 3.81 (s, 3H); LCMS: 464 [M+H]⁺, RT 2.55 min.

### Example 8

### 3-methoxy-2-[(pyridin-4-ylmethyl)amino]-N-(2,2,4,4-tetrafluoro-4H-1,3-benzodioxin-6-yl)benzamide

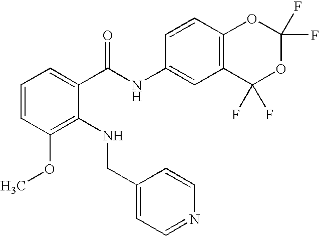

This compound was synthesized using the same synthetic route as Example 5 except that in Step 1, Intermediate E was used in place of methyl anthranylate, and in Step 2,2,2,4,4-tetrafluoro-4H-1,3-benzodioxin-6-amine was used in place of 2,2-difluoro-1,3-benzodioxol-5-amine.

¹H NMR (CD₂Cl₂-d₂) δ 8.76 (m, 2H), 8.14 (s, 1H), 7.69-7.41 (m, 3H), 7.23-7.02 (m, 5H), 5.03 (s, 1H), 4.21 (s, 2H), 3.82 (s, 3H); LCMS: 464 [M+H]⁺, RT 2.53 min.

### Example 9

### 2-methoxy-6-[(pyridin-4-ylmethyl)amino]-N-(2,2,3,3-tetrafluoro-2,3-dihydro-1,4-benzodioxin-6-yl)benzamide

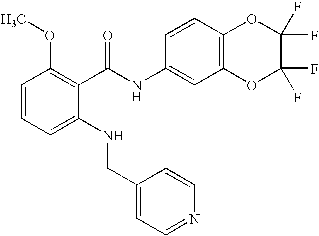

This compound was synthesized using the same synthetic route as Example 5 except that in Step 1, Intermediate F was used in place of methyl anthranylate, and in Step 2,2,2,3,3-tetrafluoro-2,3-dihydro-1,4-benzodioxin-6-amine was used in place of 2,2-difluoro-1,3-benzodioxol-5-amine.

¹H NMR (CD₂Cl₂-d₂) δ 9.95 (s, 1H), 9.25 (s, 1H), 8.52 (s, 2H), 7.79 (d, J=4.0 Hz, 1H), 7.27 (m, 3H), 7.16 (m, 2H), 6.32-6.23 (m, 2H), 4.49 (d, J=8.0 Hz, 2H), 4.0 (s, 3H); LCMS: 464 [M+H]⁺, RT 2.79 min.

### Example 10

### 2-methoxy-6-[(pyridin-4-ylmethyl)amino]-N-(2,2,4,4-tetrafluoro-4H-1,3-benzodioxin-6-yl)benzamide

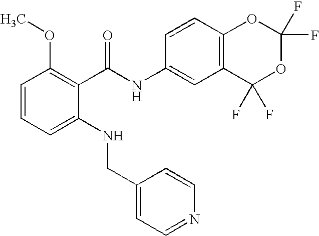

This compound was synthesized using the same synthetic route as Example 5 except that in Step 1, Intermediate F was used in place of methyl anthranylate, and in Step 2,2,2,4,4-tetrafluoro-4H-1,3-benzodioxin-6-amine was used in place of 2,2-difluoro-1,3-benzodioxol-5-amine.

¹H NMR (CD₂Cl₂-d₂) δ 10.1 (s, 1H), 9.30 (s, 1H), 8.50 (d, J=4.0 Hz, 2H), 8.12 (s, 1H), 7.73 (d, J=8.0 Hz, 1H), 7.28-7.15 (m, 4H), 6.33-6.21 (dd, J=8/40 Hz, 2H), 4.50 (d, J=4.0 Hz, 2H), 4.01 (s, 3H); LCMS: 464 [M+H]⁺, RT 2.73 min.

### Example 11

### N-(2,2-difluoro-1,3-benzodioxol-5-yl)-2-methoxy-6-[(pyridin-4-ylmethyl)amino]benzamide

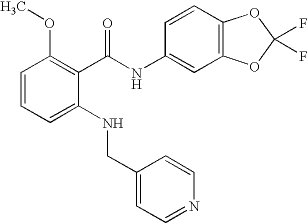

This compound was synthesized using the same synthetic route as Example 5 except that in Step 1, Intermediate F was used in place of methyl anthranylate.

¹H NMR (CD₂Cl₂-d₂) δ 9.92 (s, 1H), 9.24 (s, 1H), 8.50 (m, 2H), 7.79 (s, 1H), 7.28-7.06 (m, 5H), 6.32-6.20 (dd, J=8.0/40.0 Hz, 2H), 4.48 (d, J=4.0 Hz, 2H), 4.00 (s, 3H); LCMS: 414 [M+H]⁺, RT 2.61 min.

### Example 12

### 2-[(pyridin-4-ylmethyl)amino]-N-(2,2,3,3-tetrafluoro-2,3-dihydro-1,4-benzodioxin-6-yl)nicotinamide

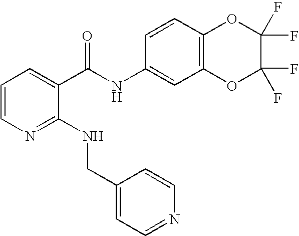

### Step 1: Preparation of 2-chloro-N-(2,2,3,3-tetrafluoro-2,3-dihydro-1,4-benzodioxin-6-yl)nicotinamide

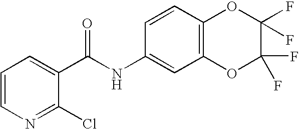

A solution of 2,2,3,3-tetrafluoro-2,3-dihydro-1,4-benzodioxin-6-amine (3.17 g, 14 mmol) in EtOAc (35 mL) was added to a flask containing 1N NaOH (28 mL). The stirred solution was treated dropwise over 30 min. with 2-chloronicotinyl chloride (3.0 g, 17 mmol) dissolved in EtOAc (3.5 mL). The reaction was stirred for 2 h until complete. The resulting solution was poured into a separatory funnel and extracted with EtOAc (3×50 mL). The combined organic layers were washed with H₂O (50 mL), 1N HCl (50 mL), H₂O (50 mL), satd NaHCO₃ (50 mL) and brine (50 mL). The organic layer was dried over MgSO₄, filtered and concentrated to afford 5.0 g of the above compound as a solid (13.8 mmol, yield 97%). ¹H NMR (DMSO-d₆) δ 11.0 (s, 1H), 8.51 to 8.53 (m, 1H), 8.06 to 8.08 (m, 1H), 7.85 (m, 1H), 7.54 to 7.57 (m, 1H), 7.45 to 7.50 (m, 2H); LCMS: 363 [M+H]⁺.

### Step 2: Preparation of 2-[(pyridin-4-ylmethyl)amino]-N-(2,2,3,3-tetrafluoro-2,3-dihydro-1,4-benzodioxin-6-yl)nicotinamide

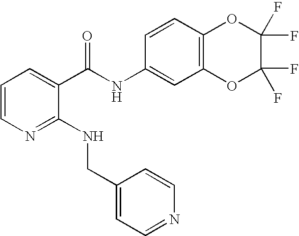

A solution of 2-chloro-N-(2,2,3,3-tetrafluoro-2,3-dihydro-1,4-benzodioxin-6-yl)nicotinamide (5.5 g, 15.2 mmol) and 4-aminomethylpyridine (3.0 mL, 30.4 mmol) were melted together at 120° C. for 48 h. The resulting solid was dissolved in EtOAc (50 mL) and satd. NaHCO₃ (50 mL) and poured into a separatory funnel. The layers were separated and the organic layer was washed with satd. NaHCO₃ (2×50 mL), H₂O (2×50 mL) and brine (50 mL). The organic layer was dried over MgSO₄, filtered and concentrated to afford a crude solid. The solid was purified by flash silica chromatography (50% EtOAc/50% Hex ramping to 100% EtOAc) to produce 5.0 g of 14 as a solid (11.5 mmol, yield 76%). ¹H NMR (DMSO-d₆) δ 10.5 (s, 1H), 8.42 to 8.44 (m, 2H), 8.37 (t, 1H), 8.13 to 8.15 (m, 1H), 8.04 to 8.06 (m, 1H), 7.88 to 7.89 to 7.50 (m, 1H), 7.55 to 7.58 (m, 1H), 7.46 to 7.48 (m, 1H), 7.25 to 7.27 (m, 2H), 6.67 to 6.70 (m, 1H), 4.66 to 4.67 (m, 2H); LCMS: 435 [M+H]⁺, RT 2.42 min.

### Example 13

### 6-methyl-2-[(pyridin-4-ylmethyl)amino]-N-(2,2,3,3-tetrafluoro-2,3-dihydro-1,4-benzodioxin-6-yl)nicotinamide

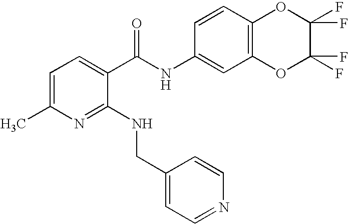

This compound was synthesized using the same synthetic route as Example 12 except that in Step 1,2-chloro-6-methylnicotinoyl chloride, formed from the corresponding carboxylic acid (as in Step 1, Example 1), was used in place of 2-chloronicotinyl chloride.

¹H NMR (MeOD-d₄) δ 8.41 (dd, J=4.32, 1.64 Hz, 2H), 7.93 (d, J=7.92 Hz, 1H), 7.81 (d, J=2.33 Hz, 1H), 7.47 (dd, J=8.94, 2.49 Hz, 1H), 7.04 to 7.42 (m, 2H), 7.23 (d, J=8.97 Hz, 1H), 6.53 (d, J=7.78 Hz, 1H), 4.80 (s, 2H), 2.32 (s, 3H); LCMS: 449 [M+H]⁺, RT 2.49 min.

### Example 14

### 4-[(pyridin-4-ylmethyl)amino]-N-(2,2,3,3-tetrafluoro-2,3-dihydro-1,4-benzodioxin-6-yl)nicotinamide

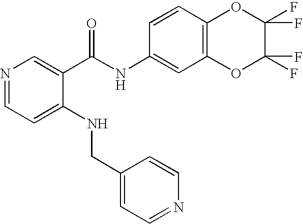

This compound was synthesized using the same synthetic route as Example 12 except that in Step 1,4-chloropyrimidine-5-carbonyl chloride, formed from the corresponding carboxylic acid (as in Step 1, Example 1), was used in place of 2-chloronicotinyl chloride.

¹H NMR (DMSO-d₆) δ 10.6 (s, 1H), 8.62 (s, 1H), 8.46 (dd, J=4.41, 1.6 Hz, 2H), 8.08 (d, J=6.1 Hz, 1H), 7.88 (d, J=2.34 Hz, 1H), 7.56 (dd, J=9.15, 2.11 Hz, 1H), 7.43 (d, J=9.3 Hz, 1H), 7.27 (d, J=5.96 Hz, 2H), 6.48 (d, J=5.97 Hz, 1H), 4.52 (d, J=6.0 Hz, 2H); LCMS: 435 [M+H]⁺, RT 1.86 min.

### Example 15

### N-(2,2-difluoro-1,3-benzodioxol-5-yl)-2-[(pyridin-4-ylmethyl)amino]nicotinamide

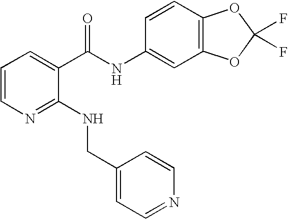

This compound was synthesized using the same synthetic route as Example 12 except that in Step 1,2,2-difluoro-1,3-benzodioxol-5-amine was used in place of 2,2,3,3-tetrafluoro-2,3-dihydro-1,4-benzodioxin-6-amine.

¹H NMR (DMSO-d₆) δ 10.4 (s, 1H), 8.41 (dd, J=4.46, 1.44 Hz, 2H), 8.36 (t, 1H), 8.11 (dd, J=4.78, 1.68 HZ, 1H), 8.03 (dd, J=7.59, 2.0 Hz, 1H), 7.80 (d, J=1.90 Hz, 1H), 7.35 to 7.41 (m, 2H), 7.23 (d, J=5.73 Hz, 2H), 6.65 (dd, J=7.63, 4.66 Hz, 1H), 4.64 (d, J=5.99 Hz, 1H); LCMS: 435 [M+H]⁺, RT 1.86 min.

### Example 16

### 2-[(Pyridin-4-ylmethyl)amino]-N-[2,4,4-trifluoro-2-(trifluoromethyl)-4H-1,3-benzodioxin-6-yl]nicotinamide

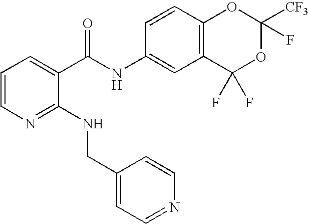

This compound was synthesized using the same synthetic route as Example 12 except that in Step 1,2,4,4-trifluoro-2-(trifluoromethyl)-4H-1,3-benzodioxin-6-amine was used in place of 2,2,3,3-tetrafluoro-2,3-dihydro-1,4-benzodioxin-6-amine.

¹H NMR (DMSO-d₆) δ 10.5 (s, 1H), 8.28 to 8.30 (m, 3H), 8.22 (d, J=2.18 Hz, 1H), 8.01 (dd, J=4.68, 1.36 Hz, 1H), 7.94 (dd, J=7.58, 1.43 Hz, 1H), 7.84 (dd, J=8.94, 1.95 Hz, 1H), 7.41 (d, J=9.12 Hz, 1H), 7.12 (d, J=5.54 Hz, 2H), 6.55 (dd, J=7.55, 5.05 Hz, 1H), 4.53 (d, J=5.98 Hz, 2H); LCMS: 485 [M+H]⁺, RT 2.76 min.

### Example 17

### 3-[(Pyridin-4-ylmethyl)amino]-N-(2,2,3,3-tetrafluoro-2,3-dihydro-1,4-benzodioxin-6-yl)pyridine-2-carboxamide

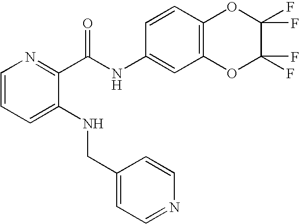

### Step 1: Preparation of 3-amino-N-(2,2,3,3-tetrafluoro-2,3-dihydro-1,4-benzodioxin-6-yl)pyridine-2-carboxamide

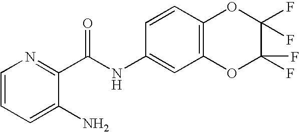

To a solution of 3-aminopicolinic acid (200 mg, 1.45 mmol) and 2,2,3,3-tetrafluoro-2,3-dihydro-1,4-benzodioxin-6-amine (323 mg, 1.45 mmol) in CH₂Cl₂ (10 mL) was added diisopropylethylamine (0.32 mL, 1.81 mmol) followed by EDCI (347 mg, 1.81 mmol) and 1-hydroxybenzotriazole (196 mg, 1.45 mmol). The reaction was stirred at room temperature for 16 h until complete. The resulting solution was concentrated and re-dissolved in 1H NaOH (10 mL) and allowed to stir at room temperature for 30 min. The solution was poured into a separatory funnel and extracted with CH₂Cl₂ (3×20 mL). The combined organic layers were dried over MgSO₄, filtered and concentrated to afford a crude yellow oil. Flash silica chromatography using 30% EtOAc/70% Hex yielded 208 mg of a clear oil (0.605 mmol, yield 42%). ¹H NMR (DMSO-d₆) δ 10.7 (s, 1H), 8.02 (d, J=2.28 Hz, 1H), 7.88 (dd, J=4.06, 1.58 Hz, 1H), 7.77 (dd, J=8.92, 2.37 Hz, 1H), 7.42 (d, J=9.05 Hz, 1H), 7.31 (dd, J=8.46, 4.14 Hz, 1H), 7.21 (dd, J=8.47, 1.44 Hz, 1H); LCMS: 344 [M+H]⁺.

### Step 2: Preparation of 3-[(pyridin-4-ylmethyl)amino]-N-(2,2,3,3-tetrafluoro-2,3-dihydro-1,4-benzodioxin-6-yl)pyridine-2-carboxamide

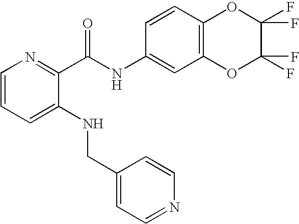

This compound was prepared similarly to Step 3 of Example 1.

¹H NMR (CDCl₃-d) δ 10.2 (s, 1H), 8.82 (t, 1H), 8.47 (dd, J=4.45, 1.58 Hz, 2H), 7.78 (dd, J=4.29, 1.52 Hz, 1H), 7.74 (d, J=2.19 Hz, 1H), 7.24 (dd, J=8.66, 2.45 Hz, 1H), 7.18 (d, J=5.92 Hz, 2H), 7.12 (dd, J=8.56, 4.3 Hz, 1H), 7.03 (d, J=8.59 Hz, 1H), 6.77 (dd, J=8.58, 1.16 Hz, 1H), 4.42 (d, J=6.0 Hz, 2H); LCMS: 435 [M+H]⁺, RT 3.21 min.

### Example 17a

### 3-[(Pyridin-4-ylmethyl)amino]-N-(2,2,3,3-tetrafluoro-2,3-dihydro-1,4-benzodioxin-6-yl)isonicotinamide

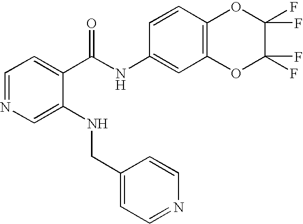

This compound can be prepared using the procedure of example 17 substituting 3-aminoisonicotinic acid for 3-aminopicolinic acid.

### Example 18

### 4-fluoro-2-[(pyridin-4-ylmethyl)amino]-N-(2,2,3,3-tetrafluoro-2,3-dihydro-1,4-benzodioxin-6-yl)benzamide

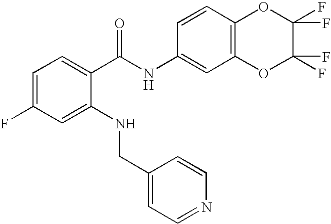

This compound was synthesized using the same synthetic route as Example 17 except that in Step 1,2-amino-4-fluorobenzoic acid was used in place of 3-aminopicolinic acid.

¹H NMR (DMSO-d₆) δ 10.46 (s, 1H) 8.75 (d, J=5.5 Hz, 2H), 8.31 (s, 2H), 7.91, (s, 1H), 7.82-7.76 (m, 3H), 7.59 (d, J=8 Hz, 1H), 7.50 (d, J=9.5 Hz, 1H), 6.51 (t, J=8 Hz, 1H), 6.37 (d, J=12 Hz, 1H), 4.72 (d, J=6 Hz, 2H); LCMS: 452 [M+H]⁺, RT 3.25 min.

### Example 19

### 5-fluoro-2-[(pyridin-4-ylmethyl)amino]-N-(2,2,3,3-tetrafluoro-2,3-dihydro-1,4-benzodioxin-6-yl)benzamide

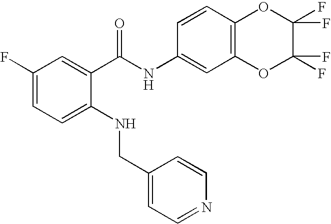

This compound was synthesized using the same synthetic route as Example 17 except that in Step 1,2-amino-5-fluorobenzoic acid was used in place of 3-aminopicolinic acid.

¹H NMR (DMSO-d₆) δ 10.46 (s, 1H) 8.75 (d, J=5.5 Hz, 2H), 8.31 (s, 2H), 7.91, (s, 1H), 7.82-7.76 (m, 3H), 7.59 (d, J=8 Hz, 1H), 7.50 (d, J=9.5 Hz, 1H), 6.51 (t, J=8 Hz, 1H), 6.37 (d, J=12 Hz, 1H), 4.72 (d, J=6 Hz, 2H); LCMS: 452 [M+H]⁺, RT 3.19 min.

### Example 20

### N-(2,2-difluoro-1,3-benzodioxol-5-yl)-5-fluoro-2-[(pyridin-4-ylmethyl)amino]benzamide

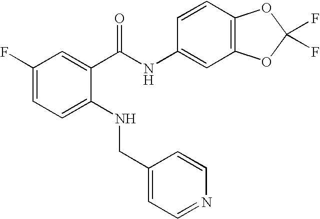

This compound was synthesized using the same synthetic route as Example 17 except that in Step 1,2-amino-5-fluorobenzoic acid was used in place of 3-aminopicolinic acid and 2,2-difluoro-1,3-benzodioxol-5-amine was used in place of 2,2,3,3-tetrafluoro-2,3-dihydro-1,4-benzodioxin-6-amine.

¹H NMR (DMSO-d₆) δ 10.33 (s, 1H) 8.44 (d, J=5 Hz, 2H), 7.80 (s, 1H), 7.70, (t, J=6 Hz, 1H), 7.53 (dd, J=3, 10, 1H), 7.43-7.33 (m, 2H), 7.27 (d, J=6 Hz, 2H), 7.11 (t, J=8 Hz, 1H), 6.49 (dd, J=5, 9 Hz, 1H), 4.42 (d, J=7 Hz, 2H); LCMS: 402 [M+H]⁺, RT 2.40 min.

### Example 21

### N-(2,2-difluoro-1,3-benzodioxol-5-yl)-4-fluoro-2-[(pyridin-4-ylmethyl)amino]benzamide

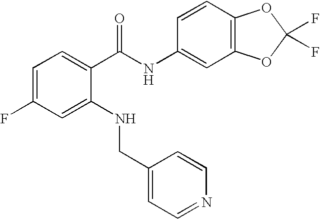

This compound was synthesized using the same synthetic route as Example 17 except that in Step 1,2-amino-4-fluorobenzoic acid was used in place of 3-aminopicolinic acid and 2,2-difluoro-1,3-benzodioxol-5-amine was used in place of 2,2,3,3-tetrafluoro-2,3-dihydro-1,4-benzodioxin-6-amine.

¹H NMR (DMSO-d₆) δ 10.33 (s, 1H) 8.68 (d, J=6 Hz, 2H), 8.28 (s, 1H), 7.79-7.70, (m, 4H), 7.42-7.34 (m, 2H), 6.46 (td, J=2.9, 1H), 6.32 (dd, J=2, 13 Hz, 1H), 4.66 (d, J=4 Hz, 2H); LCMS: 402 [M+H]⁺, RT 2.28 min.

### Example 22

### 2-{[(2-cyanopyridin-4-yl)methyl]amino}-N-(2,2,3,3-tetrafluoro-2,3-dihydro-1,4-benzodioxin-6-yl)benzamide

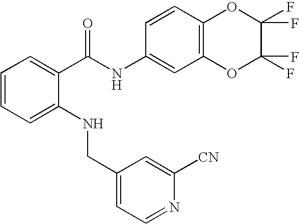

### Step 1: Preparation of 2-nitro-N-(2,2,3,3-tetrafluoro-2,3-dihydro-1,4-benzodioxin-6-yl)benzamide

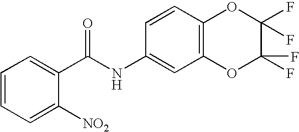 [0469] This material is prepared in Example 3 Step 1.

### Step 2: Preparation of 2-amino-N-(2,2,3,3-tetrafluoro-2,3-dihydro-1,4-benzodioxin-6-yl)benzamide

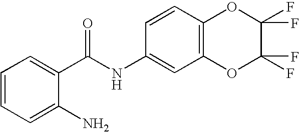

This material is prepared in Example 3 Step 2.

### Step 3: Preparation of 2-{[(2-cyanopyridin-4-yl)methyl]amino}-N-(2,2,3,3-tetrafluoro-2,3-dihydro-1,4-benzodioxin-6-yl)benzamide

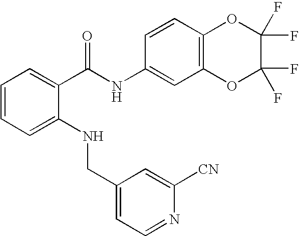

A solution of Intermediate B (148.8 mg, 0.7 mmol) in 2 mL THF was added over 8 h to a solution of 2-amino-N-(2,2,3,3-tetrafluoro-2,3-dihydro-1,4-benzodioxin-6-yl)benzamide (150 mg, 0.44 mmol) (Product from example 3 step 2), potassium carbonate (132 mg, 0.96 mmol) and sodium iodide (65.7 mg, 0.44 mmol) in THF (3 mL) at 45° C. After stirring an additional 10 h the reaction was diluted with EtOAc (30 mL) and washed with water and brine. The organic layer was dried with sodium sulfate, filtered and concentrated to provide a yellow solid. The solid was purified by silica chromatography (50% EtOAc/50% Hex) to provide 127 mg of the above compound as a white solid (0.277 mmol, 63%). ¹H NMR (DMSO-d₆) δ 10.47 (s, 1H), 8.67 (dd, J=5.1, 1 Hz, 1H), 7.97 (d, J=1 Hz, 1H), 7.94 (d, J=2.5 Hz, 1H), 7.84-7.88 (m, 1H), &.60 (d, J=2.5 Hz, 1H), 7.48 (d, J=9 Hz, 1H), 7.26 (at, J=7.5 Hz, 1H), 6.69 (at, J=7.5 Hz, 1H), 6.52 (d, J=8 Hz, 1H), 4.57 (d, J=6 Hz, 2H); LCMS: 458.9 [M+H]⁺, RT 3.78 min; TLC R_{f}=0.35 (40% EtOAc in Hex).

### Example 23

### 2-{[(2-cyanopyridin-4-yl)methyl]amino}-N-(2,2,4,4-tetrafluoro-4H-1,3-benzodioxin-6-yl)benzamide

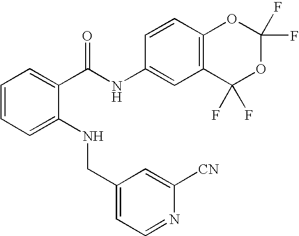

This compound was synthesized using the same synthetic route as Example 22 except that in Step 1,2,2,4,4-tetrafluoro-4H-1,3-benzodioxin-6-amine was used in place of 2,2,3,3-tetrafluoro-2,3-dihydro-1,4-benzodioxin-6-amine.

¹H NMR (DMSO-d₆) δ 10.55 (s, 1H), 8.67 (dd, J=5, 1 Hz, 1H), 8.41 (d, J=2.5 Hz, 1H), 7.91-8.01 (m, 3H), 7.71 (dd, J=8, 1.5 Hz, 1H), 7.66 (dd, J=5, 1.8 Hz, 1H), 7.22-7.28 (m, 1H), 6.66-6.72 (m, 1H), 4.58 (d, J=6 Hz, 2H); LCMS: 459 [M+H]⁺, RT 3.65 min; TLC R_{f}=0.40 (40% EtOAc in Hex).

### Example 24

### 4-{[(2-{[(2,2-difluoro-1,3-benzodioxol-5-yl)amino]carbonyl}phenyl)amino]methyl}-N-methylpyridine-2-carboxamide

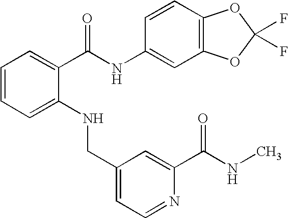

This compound was synthesized using the same synthetic route as Example 22 except that in Step 1,2,2-difluoro-1,3-benzodioxol-5-amine was used in place 2,2,3,3-tetrafluoro-2,3-dihydro-1,4-benzodioxin-6-amine, and in Step 3, Intermediate A is used in place of Intermediate B.

¹H NMR (DMSO-d₆) δ 10.34 (s, 1H) 8.72 (d, J=5 Hz, 1H), 8.52 (d, J=6 Hz, 1H), 7.98-7.93, (m, 2H), 7.85 (d, J=2 Hz, 1H), 7.66 (d, J=7 Hz, 1H), 7.50 (d, J=5 Hz, 1H), 7.45 (d, J=9 Hz, 1H), 7.36 (d, J=8 Hz, 1H), 7.21 (t, J=8 Hz, 1H), 6.63 (t, J=7 Hz, 1H), 6.49 (d, J=9 Hz, 1H), 4.56 (d, J=8 Hz, 2H), 2.78 (d, J=5 Hz, 3H); LCMS: 441 [M+H]⁺, RT 3.39 min.

### Example 25

### 2-{[(2-cyanopyridin-4-yl)methyl]amino}-N-(2,2-difluoro-1,3-benzodioxol-5-yl)benzamide

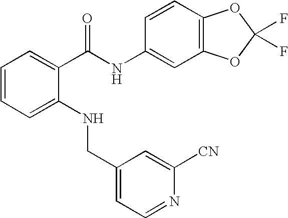

This compound was synthesized using the same synthetic route as Example 22 except that in Step 1,2,2-difluoro-1,3-benzodioxol-5-amine was used in place of 2,2,3,3-tetrafluoro-2,3-dihydro-1,4-benzodioxin-6-amine.

¹H NMR (DMSO-d₆) δ 10.35 (s, 1H) 8.65 (d, J=5 Hz, 1H), 7.94 (s, 1H), 7.88-7.85, (m, 2H), 7.66-7.62 (m, 2H), 7.44 (dd, J=2, 9 Hz, 1H), 7.37 (d, J=9 Hz, 1H), 7.22 (t, J=8 Hz, 1H), 6.65 (t, J=8 Hz, 1H), 6.48 (d, J=9 Hz, 1H), 4.55 (d, J=8 Hz, 2H); LCMS: 409 [M+H]⁺, RT 3.60 min.

### Example 26

### 2-{[(2-chloro-6-methylpyrimidin-4-yl)methyl]amino}-N-(2,2,3,3-tetrafluoro-2,3-dihydro-1,4-benzodioxin-6-yl)benzamide

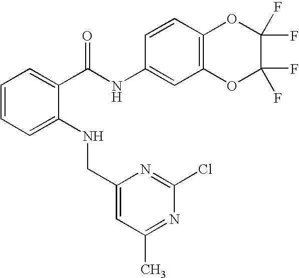

This compound was synthesized using the same synthetic route as Example 22 except that in Step 3, (2-chloro-6-methylpyrimidin-4-yl)methyl methanesulfonate (Intermediate X) was used in place of Intermediate B.

¹H NMR (CDCl₃-d) δ 7.82 (br s, 1H), 7.60 (d, J=2.39 Hz, 1H), 7.43 (dd, J=7.7, 1.51 Hz, 1H), 7.19 to 7.24 (m, 1H), 7.13 (dd, J=9.15, 2.44 Hz, 1H), 7.04 to 7.07 (m, 2H), 6.62 to 6.66 (m, 1H), 6.40 (dd, J=8.56, 0.8 Hz, 1H), 4.41 (s, 2H); LCMS: 483 [M+H]⁺, RT 4.02 min.

### Example 27

### 2-{[(2-cyanopyridin-4-yl)methyl]amino}-5-fluoro-N-(2,2,3,3-tetrafluoro-2,3-dihydro-1,4-benzodioxin-6-yl)benzamide

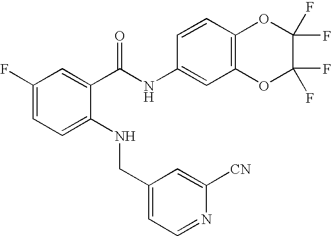

### Step 1: Preparation of 5-fluoro-2-nitro-N-(2,2,3,3-tetrafluoro-2,3-dihydro-1,4-benzodioxin-6-yl)benzamide

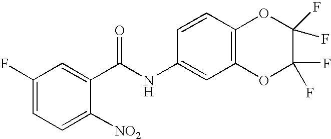

This compound was made using the procedure of Example 17 step 1 except that 2-nitro-5-fluorobenzoic acid was used in place of 3-amino-picolinic acid.

¹H NMR (DMSO-d₆) δ 11.03 (s, 1H) 8.27 (dd, J=5, 5 Hz, 1H), 7.79-7.75 (m, 2H), 7.65-7.59, (m, 1H), 7.50-7.42 (m, 2H); LCMS: 391 [M+H]⁺, RT 3.41 min.

### Step 2: Preparation of 2-amino-5-fluoro-N-(2,2,3,3-tetrafluoro-2,3-dihydro-1,4-benzodioxin-6-yl)benzamide

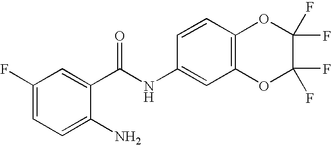

This compound was made using the procedure of Example 1, step 2 except that the product of step 1 above was used in place of 5-methoxy-2-nitro-N-(2,2,3,3-tetrafluoro-2,3-dihydro-1,4-benzodioxin-6-yl)benzamide.

¹H NMR (DMSO-d₆) δ 10.27 (s, 1H), 7.86 (d, J=2 Hz, 1H), 7.57 (d, J=10 Hz, 1H), 7.49-7.43, (m, 2H), 7.12 (t, J=7 Hz, 1H), 6.75 (dd, J=4, 8 Hz, 1H), 6.25 (s, 2H); LCMS: 361 [M+H]⁺, RT 3.90 min.

### Step 3: Preparation of 2-{[(2-cyanopyridin-4-yl)methyl]amino}-5-fluoro-N-(2,2,3,3-tetrafluoro-2,3-dihydro-1,4-benzodioxin-6-yl)benzamide

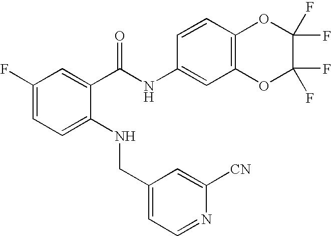

This compound was made using the procedure of Example 22, step 3 except that the product of step 2 above was used instead of 2-amino-N-(2,2,3,3-tetrafluoro-2,3-dihydro-1,4-benzodioxin-6-yl)benzamide.

¹H NMR (CD₂Cl₂-d₂) δ 8.64 (d, J=4.9 Hz, 1H), 7.91 (s, 1H), 7.75, (d, J=2.0 Hz, 1H), 7.70 (s, 1H), 7.53 (d, J=2.0 Hz, 1H), 7.34-7.18 (m, 3H), 7.10-7.03 (m, 1H), 6.41 (dd, J=5, 9 Hz, 1H), 4.52 (s, 2H); LCMS: 477 [M+H]⁺, RT 3.70 min.

### Example 28

### 2-{[(2-cyanopyridin-4-yl)methyl]amino}-4-fluoro-N-(2,2,3,3-tetrafluoro-2,3-dihydro-1,4-benzodioxin-6-yl)benzamide

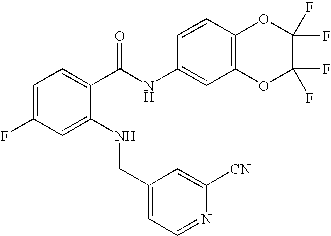

This compound was synthesized using the same synthetic route as Example 27 except in Step 1,4-fluoro-2-nitrobenzoic acid was used in place of 5-fluoro-2-nitrobenzoic acid.

¹H NMR (CD₂Cl₂-d₂) δ 8.64 (d, J=4.9 Hz, 1H), 8.47 (s, 1H), 7.87 (s, 1H), 7.73 (d, J=2.0 Hz, 1H), 7.69 (s, 1H), 7.59 (dd, J=7.0, 8.0 Hz, 1H), 7.52 (d, J=5 Hz, 1H), 6.46 (t, J=8 Hz, 1H), 6.13 (dd, J=2, 11 Hz, 1H), 4.53 (s, 2H); LCMS: 477 [M+H]⁺, RT 3.71 min.

### Example 29

### 2-{[(2-cyanopyridin-4-yl)methyl]amino}-N-(2,2-difluoro-1,3-benzodioxol-5-yl)-4-fluorobenzamide

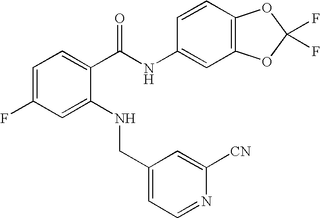

This compound was synthesized using the same synthetic route as Example 27 except in Step 1,4-fluoro-2-nitrobenzoic acid is used in place of 5-fluoro-2-nitrobenzoic acid and 2,2-difluoro-1,3-benzodioxol-5-amine is used in place of 2,2,3,3-tetrafluoro-2,3-dihydro-1,4-benzodioxin-6-amine.

¹H NMR (DMSO-d₆) δ 10.30 (s, 1H) 8.64 (d, J=7 Hz, 2H), 8.18 (t, J=6, 1H), 7.92, (s, 1H), 7.80 (d, J=3 Hz, 1H), 7.72 (dd, J=7, 9 Hz, 1H), 7.61 (d, J=6 Hz, 1H), 7.41-7.33 (m, 2H), 6.44 (td, J=2, 10 Hz, 1H), 6.31 (dd, J=2, 12 Hz, 1H), 4.35 (d, J=6 Hz, 2H); LCMS: 427 [M+H]⁺, RT 3.55 min.

### Example 30

### 2-{[(2-cyanopyridin-4-yl)methyl]amino}-N-(2,2-difluoro-1,3-benzodioxol-5-yl)-5-fluorobenzamide

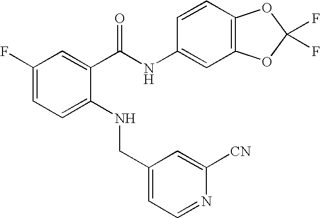

This compound was synthesized using the same synthetic route as Example 27 except in Step 1,2,2-difluoro-1,3-benzodioxol-5-amine is used in place of 2,2,3,3-tetrafluoro-2,3-dihydro-1,4-benzodioxin-6-amine.

¹H NMR (DMSO-d₆) δ 10.35 (s, 1H) 8.61 (d, J=5 Hz, 2H), 7.90 (s, 1H), 7.81, (s, 1H), 7.68 (t, J=6 Hz, 1H), 7.59 (d, J=5 Hz, 1H), 7.51 (dd, J=3, 11 Hz, 1H), 7.41-7.33 (m, 2H), 7.09 (td, J=3, 10 Hz, 1H), 6.45 (dd, J=4, 9 Hz, 1H), 4.50 (d, J=7 Hz, 2H); LCMS: 427 [M+H]⁺, RT 3.57 min.

### Example 31

### 4-{[(2-{[(2,2-difluoro-1,3-benzodioxol-5-yl)amino]carbonyl}-4-fluorophenyl)amino]methyl}-N-methylpyridine-2-carboxamide

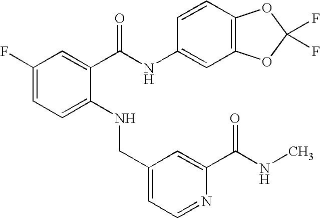

This compound was synthesized using the same synthetic route as Example 27 except in Step 1,2,2-difluoro-1,3-benzodioxol-5-amine is used in place of 2,2,3,3-tetrafluoro-2,3-dihydro-1,4-benzodioxin-6-amine, and in Step 3, Intermediate A was used in place of Intermediate B.

¹H NMR (DMSO-d₆) δ 10.38 (s, 1H) 8.75 (d, J=5 Hz, 1H), 8.54 (d, J=6 Hz, 1H), 7.96, (s, 1H), 7.85 (s, 1H), 7.80 (t, J=6 Hz, 1H), 7.55 (d, J=9 Hz, 1H), 7.50-7.38 (m, 3H), 7.13 (t, J=9 Hz, 1H), 6.49 (dd, J=6, 9 Hz, 1H), 4.56 (d, J=7 Hz, 2H), 2.78 (d, J=6 Hz, 3H); LCMS: 459 [M+H]⁺, RT 3.35 min.

### Example 32

### 4-{[(5-fluoro-2-{[(2,2,3,3-tetrafluoro-2,3-dihydro-1,4-benzodioxin-6-yl)amino]carbonyl}phenyl)amino]methyl}-N-methylpyridine-2-carboxamide

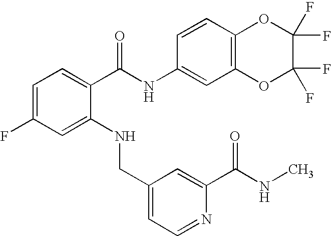

This compound was synthesized using the same synthetic route as Example 27 except in Step 1,4-fluoro-2-nitrobenzoic acid is used in place of 5-fluoro-2-nitrobenzoic acid, and in Step 3, {2-[(methylamino)carbonyl]pyridin-4-yl}methyl methanesulfonate (Intermediate A) was used in place of (2-cyanopyridin-4-yl)methyl methanesulfonate (Intermediate B).

¹H NMR (DMSO-d₆) δ 10.42 (s, 1H) 8.75 (d, J=5 Hz, 2H), 8.54 (d, J=5 Hz, 1H), 8.26, (t, J=6 Hz, 1H), 7.97 (s, 1H), 7.88 (d, J=2 Hz, 1H), 7.76 (dd, J=7, 9 Hz, 1H), 7.58 (dd, J=2, 9 Hz, 1H), 7.51 (d, J=5 Hz, 1H), 7.46 (d, J=10 Hz, 1H), 6.47 (td, J=3, 9 Hz, 1H), 6.33 (dd, J=2, 12 Hz, 1H), 4.58 (d, J=7 Hz, 2H2.78 (d, J=6 Hz, 3H); LCMS: 509 [M+H]⁺, RT 3.75 min.

### Example 33

### 4-{[(2-{[(2,2-difluoro-1,3-benzodioxol-5-yl)amino]carbonyl}-5-fluorophenyl)amino]methyl}-N-methylpyridine-2-carboxamide

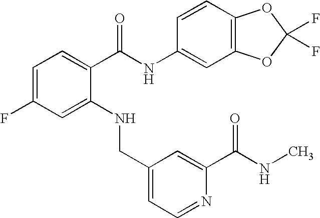

This compound was synthesized using the same synthetic route as Example 27 except in Step 1,4-fluoro-2-nitrobenzoic acid is used in place of 5-fluoro-2-nitrobenzoic acid and 2,2-difluoro-1,3-benzodioxol-5-amine is used in place of 2,2,3,3-tetrafluoro-2,3-dihydro-1,4-benzodioxin-6-amine, and in Step 3, Intermediate A was used in place of Intermediate B.

¹H NMR (DMSO-d₆) δ 10.30 (s, 1H) 8.75 (d, J=5 Hz, 2H), 8.54 (d, J=5 Hz, 1H), 8.29, (t, J=6 Hz, 1H), 7.97 (s, 1H), 7.83 (d, J=2 Hz, 1H), 7.79-7.74 (m, 1H), 7.50 (d, J=6 Hz, 1H), 7.42-7.37 (m, 2H), 6.45 (t, J=8 Hz, 1H), 6.34 (d, J=11 Hz, 1H), 4.58 (d, J=5 Hz, 2H), 2.78 (d, J=6 Hz, 3H); LCMS: 459 [M+H]⁺, RT 3.45 min.

### Example 34

### 4-{[(4-fluoro-2-{[(2,2,3,3-tetrafluoro-2,3-dihydro-1,4-benzodioxin-6-yl)amino]carbonyl}phenyl)amino]methyl}-N-methylpyridine-2-carboxamide

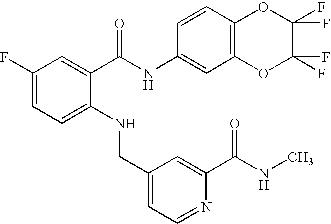

This compound was synthesized using the same synthetic route as Example 27 except in Step 3, Intermediate A was used in place of Intermediate B.

¹H NMR (DMSO-d₆) δ 10.45 (s, 1H) 8.75 (d, J=5 Hz, 1H), 8.52 (d, J=6 Hz, 1H), 7.96, (s, 1H), 7.90 (d, J=3 Hz, 1H), 7.78 (t, J=6 Hz, 1H), 7.61-7.54 (m, 2H), 7.50-7.45 (m, 2H), 7.13 (td, J=3, 9 Hz, 1H), 6.49 (dd, J=6, 8 Hz, 1H), 4.56 (d, J=6 Hz, 2H), 2.78 (d, J=4 Hz, 3H); LCMS: 509 [M+H]⁺, RT 3.73 min.

### Example 35

### 4-{[(2-{[(2,2-difluoro-1,3-benzodioxol-5-yl)amino]carbonyl}-4,5-difluorophenyl)amino]methyl}-N-methylpyridine-2-carboxamide

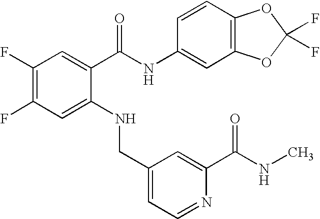

### Step 1: Preparation of 2-amino-N-(2,2-difluoro-1,3-benzodioxol-5-yl)-4,5-difluorobenzamide

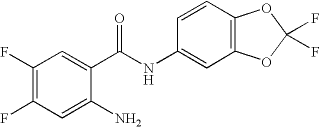

This compound was prepared as in Example 17, Step 1 except 2-amino-4,5-difluorobenzoic acid was used in place of 3-aminopicolinic acid and 2,2-difluoro-1,3-benzodioxol-5-amine was used in place of 2,2,3,3-tetrafluoro-2,3-dihydro-1,4-benzodioxin-6-amine.

### Step 2: Preparation of The Title Compound

This compound was prepared as in Example 22, Step 3 except that the product of step 1 above was used in place of 2-amino-N-(2,2,3,3-tetrafluoro-2,3-dihydro-1,4-benzodioxin-6-yl)benzamide and Intermediate A was used in place of Intermediate B.

¹H NMR (DMSO-d₆) δ 10.33 (s, 1H) 8.75 (d, J=4 Hz, 2H), 8.54 (d, J=5 Hz, 1H), 8.13, (t, J=6 Hz, 1H), 7.88-7.81 (m, 2H), 7.50 (d, J=5 Hz, 1H), 7.41 (s, 2H), 6.57 (dd, J=6, 7 Hz, 1H), 4.57 (d, J=6 Hz, 2H), 2.78 (d, J=6 Hz, 3H); LCMS: 477 [M+H]⁺, RT 3.53 min.

### Example 36

### 2-{[(2-cyanopyridin-4-yl)methyl]amino}-N-(2,2-difluoro-1,3-benzodioxol-5-yl)-4,5-difluorobenzamide

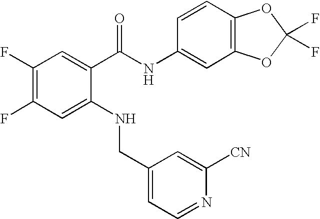

This compound was synthesized using the same synthetic route as Example 35 except in Step 2, Intermediate B was used in place of Intermediate A.

¹H NMR (DMSO-d₆) δ 10.33 (s, 1H) 8.66 (d, J=4 Hz, 2H), 8.07, (s, 1H), 7.94 (s, 1H), 7.87-7.79 (m, 1H), 7.62 (d, J=7 Hz, 1H), 7.40 (s, 2H), 6.57 (dd, J=6, 7 Hz, 1H), 4.55 (d, J=11 Hz, 2H); LCMS: 445 [M+H]⁺, RT 3.78 min.

### Example 37

### 4-{[(4,5-difluoro-2-{[(2,2,3,3-tetrafluoro-2,3-dihydro-1,4-benzodioxin-6-yl)amino]carbonyl}phenyl)amino]methyl}-N-methylpyridine-2-carboxamide

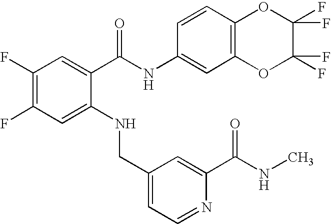

This compound was synthesized using the same synthetic route as Example 35 except in Step 1,2,2,3,3-tetrafluoro-2,3-dihydro-1,4-benzodioxin-6-amine was used in place 2,2-difluoro-1,3-benzodioxol-5-amine.

¹H NMR (DMSO-d₆) δ 10.42 (s, 1H) 8.75 (d, J=5 Hz, 2H), 8.55 (d, J=5 Hz, 1H), 8.13, (t, J=6 Hz, 1H), 7.98 (s, 1H), 7.89-7.81 (m, 2H), 7.59-7.46 (m, 3H), 6.59 (dd, J=6, 7 Hz, 1H), 4.57 (d, J=8 Hz, 2H), 2.78 (d, J=6 Hz, 3H); LCMS: 527 [M+H]⁺, RT 3.77 min.

### Example 38

### 2-{[(2-cyanopyridin-4-yl)methyl]amino}-4,5-difluoro-N-(2,2,3,3-tetrafluoro-2,3-dihydro-1,4-benzodioxin-6-yl)benzamide

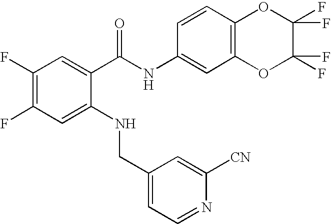

This compound was synthesized using the same synthetic route as Example 35 except in Step 1,2,2,3,3-tetrafluoro-2,3-dihydro-1,4-benzodioxin-6-amine was used in place 2,2-difluoro-1,3-benzodioxol-5-amine, and in Step 2, Intermediate B was used in place of Intermediate A.

¹H NMR (DMSO-d₆) δ 10.41 (s, 1H) 8.66 (d, J=5 Hz, 2H), 8.04 (s, 1H), 7.94, (s, 1H), 7.88-7.81 (m, 3H), 7.62 (d, J=6 Hz, 1H), 7.56 (d, J=9 Hz, 1H), 7.47 (d, J=9 Hz, 1H), 6.59 (dd, J=6, 7 Hz, 1H), 4.56 (d, J=7 Hz, 2H); LCMS: 495 [M+H]⁺, RT 4.26 min.

### Example 39

### 5-[(pyridin-4-ylmethyl)amino]-N-(2,2,3,3-tetrafluoro-2,3-dihydro-1,4-benzodioxin-6-yl)pyrimidine-4-carboxamide

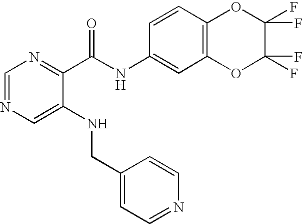

### Step 1: Preparation of 5-bromo-2-(methylthio)-N-(2,2,3,3-tetrafluoro-2,3-dihydro-1,4-benzodioxin-6-yl)pyrimidine-4-carboxamide

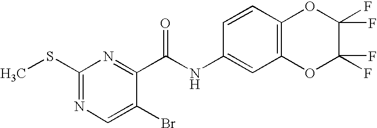

5-Bromo-2-(methylthio)pyrimidine-4-carboxylic acid (0.5 g, 2.01 mmol), 2,2,3,3-tetrafluoro-2,3-dihydro-1,4-benzodioxin-6-amine (0.45 g, 2.01 mmol), EDAC (0.48 g, 2.51 mmol), 1-hydroxybenzotriazole (0.27 g, 2.01 mmol) and diisopropylethylamine (0.32 g, 2.51 mmol) were combined in a mixture of CH₂Cl₂ (10 mL) and THF (10 mL). This solution was allowed to stir at RT for 5 days. The reaction was diluted with EtOAc and washed with sat. aq. NaHCO₃ and then brine. The organic layer was dried over MgSO₄, filtered and concentrated to afford a crude solid. The solid was purified by flash silica chromatography (EtOAc/hexanes 4:6) to produce the title compound as a solid (0.7 g, 77%). ¹H NMR (CD₂Cl₂-d₂) δ 9.70 (s, 1H), 8.85 (s, 1H), 7.87 (s, 1H), 7.43 (d, J=8.0 Hz, 2H), 7.20 (d, J=8.0 Hz, 2H), 2.65 (s, 3H); LCMS: 455 [M+H]⁺, RT 4.21 min.

### Step 2: Preparation of 2-(methylthio)-5-[(pyridin-4-ylmethyl)amino]-N-(2,2,3,3-tetrafluoro-2,3-dihydro-1,4-benzodioxin-6-yl)pyrimidine-4-carboxamide

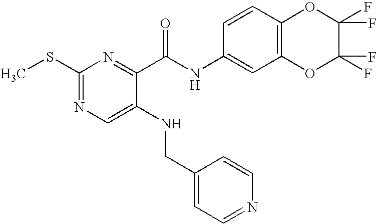

A mixture of 5-bromo-2-(methylthio)-N-(2,2,3,3-tetrafluoro-2,3-dihydro-1,4-benzodioxin-6-yl)pyrimidine-4-carboxamide (0.1 g, 0.22 mmol), 4-(methylamino)pyridine (0.14 mL, 1.32 mmol), CuO (88.0 mg, 0.01 mmol) and K₂CO₃ (37.0 mg, 0.26 mmol) was heated to 90° C. in DMF (1.0 mL) until the reaction was complete by TLC. The reaction was cooled down to RT and evaporated to dryness, taken up in CH₂Cl₂ and purified via flash silica chromatography (Acetone/CH₂Cl₂ 1:3) to produce the title compound as a solid (36.0 mg, 34%). ¹H NMR (CD₂Cl₂-d₂) δ 9.88 (s, 1H), 8.45 (s, 2H), 8.20 (s, 1H), 8.02 (s, 1H), 7.85 (s, 1H), 7.72 (s, 1H), 7.30 (s, 2H), 7.10 (s, 1H), 4.48 (d, J=4.0 Hz, 2H), 2.62 (s, 3H); LCMS: 482 [M+H]⁺, RT 2.87 min.

### Step 3: Preparation of 5-[(pyridin-4-ylmethyl)amino]-N-(2,2,3,3-tetrafluoro-2,3-dihydro-1,4-benzodioxin-6-yl)pyrimidine-4-carboxamide

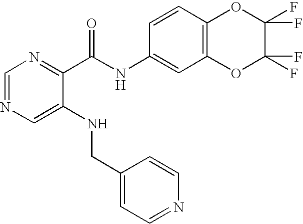

A small amount of wet Raney Nickel was added to a flask and this was triturated with absolute EtOH (3×). A solution of 2-(methylthio)-5-[(pyridin-4-ylmethyl)amino]-N-(2,2,3,3-tetrafluoro-2,3-dihydro-1,4-benzodioxin-6-yl)pyrimidine-4-carboxamide (80 mg, 0.17 mmol) in EtOH (1 mL) was added to the flask and the mixture was allowed to stir at reflux overnight under an atmosphere of nitrogen. Upon completion, the reaction was filtered through a Celite pad that was rinsed well with EtOH. Volatiles were evaporated and the crude oil was purified via flash column chromatography using 5% MeOH/95% CH₂Cl₂ to give the product (25 mg, 35%). ¹H NMR (CD₂Cl₂-d₂) δ 10.1 (s, 1H), 8.47 to 8.42 (m, 4H), 8.14 (s, 1H), 7.77 (s, 1H), 7.29-7.22 (m, 3H), 7.10 (d, J=8.0 Hz, 1H), 4.51 (d, J=8.0 Hz, 2H); LCMS: 436 [M+H]⁺, RT 2.50 min.

### Example 40

### 2-({[2-(4,5-dihydro-1H-imidazol-2-yl)pyridin-4-yl]methyl}amino)-N-(2,2,4,4-tetrafluoro-4H-1,3-benzodioxin-6-yl)benzamide

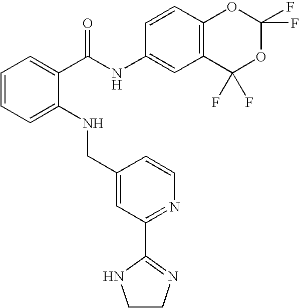

To a solution of 2-{[(2-cyanopyridin-4-yl)methyl]amino}-N-(2,2,4,4-tetrafluoro-4H-1,3-benzodioxin-6-yl)benzamide (Example 23, 0.23 g, 0.51 mmol) and ethylene diamine (0.10 mL, 1.54 mmol) in DMF (5.0 mL) was added pieces of elemental sulfur (49.3 mg, 1.54 mmol) and the reaction was heated at 110° C. until completion as observed by TLC. The reaction was evaporated to dryness and taken up in DCM and satd. NaHCO₃ and poured into a separatory funnel. The layers were separated and the organic layer was washed with satd. NaHCO₃, H₂O and brine. The organic layer was dried over MgSO₄, filtered and concentrated to afford a crude oil that is purified via HPLC (10-90% ACN/water) to give 45 as a yellow oil (65 mg, 25%). ¹H NMR (CD₂Cl₂-d₂) δ 9.70 (s, 1H), 8.35 (d, J=4.0 Hz, 1H), 8.05-7.94 (m, 3H), 7.65 (d, J=8.0 Hz, 1H), 7.46 (d, J=8 Hz, 1H), 7.23 (d, J=4.0 Hz, 1H), 7.10-7.00 (m, 2H), 6.51-6.45 (m, 1H), 6.33 (d, J=8.0 Hz, 1H), 4.37 (d, J=4.0 Hz, 2H), 3.62 (s, 4H); LCMS: 502 [M+H]⁺, RT 2.85 min.

### Example 41

### N-(2,2-difluoro-1,3-benzodioxol-5-yl)-2-({[2-(4,5-dihydro-1H-imidazol-2-yl)pyridin-4-yl]methyl}amino)-5-fluorobenzamide

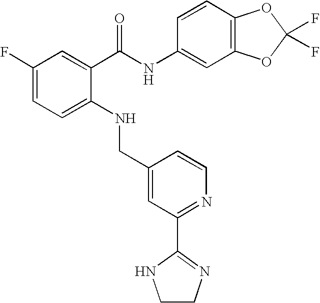

This compound was synthesized using the same synthetic route as exemplified in Example 40 except Example 30 was used as starting material in place of Example 23.

¹H NMR (CD₂Cl₂-d₂) δ 9.10 (s, 1H), 8.38 (d, J=4.0 Hz, 1H), 8.03 (s, 1H), 7.71 (s, 1H), 7.60 (s, 1H), 7.26-7.10 (m, 3H), 6.91 (d, J=8.0 Hz, 1H), 6.75 (s, 1H), 6.24-6.20 (m, 1H), 4.35 (d, J=4.0 Hz, 2H), 3.66 (s, 4H); LCMS: 470 [M+H]⁺, RT 2.69 min.

### Example 42

### 4-{[(2-{[(2,2-difluoro-1,3-benzodioxol-5-yl)amino]carbonyl}-4-fluorophenyl)amino]methyl}-N,N-dimethylpyridine-2-carboxamide

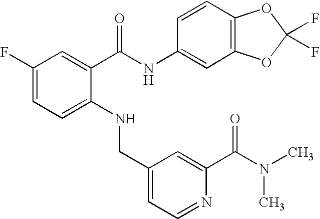

### Step 1: Preparation of methyl 2-[(dimethylamino)carbonyl]isonicotinate

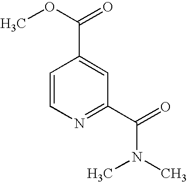

Intermediate D (560 mg, 2.87 mmol) was taken up with dichloromethane (6 mL) and allowed to stir for 15 min until all of the diester was in solution. The solution was then cooled to 0° C. and magnesium chloride (174.84 mg, 1.84 mmol) was added. This was allowed to stir for 30 min at 0° C. To the reaction vessel was then added dimethylamine (2M, 2.15 mL) over the course of 3 h. The reaction mixture was allowed to stir 16 h at rt. The reaction was then quenched with water (5 mL) and aqueous monobasic potassium phosphate (1M, 5 mL). The solution was extracted using CH₂Cl₂ (3×10 mL) and the organic fractions were combined, dried and concentrated to a white solid (525 mg, 88%). ¹H NMR showed the solid to be the correct compound in roughly 90% purity. The crude product was used without further purification. ¹H NMR (DMSO-d₆) δ 8.77 (d, J=5 Hz, 1H) 7.91 (s, 1H), 7.27, (d, J=5 Hz, 1H), 3.88 (s, 3H), 3.99 (s, 3H), 2.91 (s, 3H); LCMS: 209 [M+H]⁺, RT 1.13 min.

### Step 2: Preparation of 4-(hydroxymethyl)-N,N-dimethylpyridine-2-carboxamide

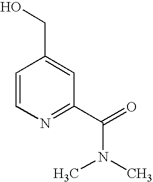

2-Dimethylcarbamoyl-isonicotinic acid methyl ester (140.00 mg, 0.67 mmol) was dissolved in 1,4-dioxane (1.16 mL). MeOH (0.18 mL) and water (0.01 mL) were then added and the solution was allowed to stir for 15 minutes. The solution was then cooled to 0° C. and sodium borohydride (31.80 mg, 0.84 mmol) was added portion-wise over the course of 1 h. The mixture was allowed to stir for 16 h. The crude reaction mixture was then added directly to a Biotage® silica samplet cartridge and dried under vacuum for 3 h. The sample was then flashed (5% MeOH in EtOAc) to yield 79.2 mg (64.8%) of the product as an oil. ¹H NMR (CD₂Cl₂-d₂) δ 8.49 (d, J=7 Hz, 1H) 7.41 (d, J=7 Hz, 2H), 7.79, (d, J=5 Hz, 2H), 3.09 (s, 3H), 2.76 (s, 3H); LCMS: 181 [M+H]⁺, RT 0.95 min.

### Step 3: Preparation of {2-[(dimethylamino)carbonyl]pyridin-4-yl}methyl methanesulfonate

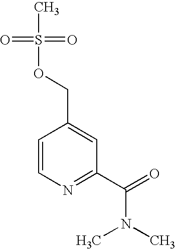

The title compound was synthesized using the same procedure as Intermediate A, substituting 4-(hydroxymethyl)-N,N-dimethylpyridine-2-carboxamide for 4-(hydroxymethyl)-N-methylpyridine-2-carboxamide. The crude reaction mixture was taken directly to the next step without purification.

### Step 4: Preparation of 4-{[(2-{[(2,2-difluoro-1,3-benzodioxol-5-yl)amino]carbonyl}-4-fluorophenyl)amino]methyl}-N,N-dimethylpyridine-2-carboxamide

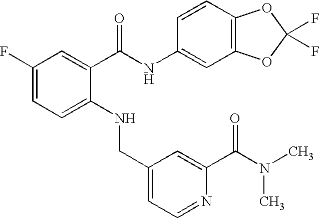

This compound was prepared using the 2-amino-N-(2,2-difluoro-1,3-benzodioxol-5-yl)-5-fluorobenzamide from the preparation of Example 30 rather than 2-amino-N-(2,2,3,3-tetrafluoro-2,3-dihydro-1,4-benzodioxin-6-yl)benzamide and the procedure from Example 22 Step 3 except {2-[(dimethylamino)carbonyl]pyridin-4-yl}methyl methanesulfonate (from Step 3 above) was used in place of Intermediate B.

¹H NMR (CD₂Cl₂-d₂) δ 8.41 (s, 1H), 8.28 (s, 1H) 7.58 (d, J=3 Hz, 1H), 7.23 (d, J=4 Hz, 1H), 7.20, (d, J=4 Hz, 1H), 7.04 (dd, J=3, 1 Hz, 1H), 6.97-6.87 (m, 2H), 6.35 (dd, J=5, 5 Hz, 1H), 4.36 (s, 2H), 2.98 (s, 3H), 2.92 (s, 3H); LCMS: 473 [M+H]⁺, RT 2.59 min.

### Example 43

### 4-{[(4,5-difluoro-2-{[(2,2,3,3-tetrafluoro-2,3-dihydro-1,4-benzodioxin-6-yl)amino]carbonyl}phenyl)amino]methyl}-N-ethylpyridine-2-carboxamide

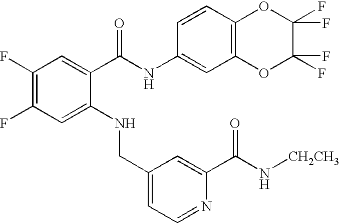

### Step 1: Preparation of {2-[(ethylamino)carbonyl]pyridin-4-yl}methyl methanesulfonate

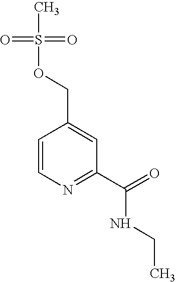

The title compound was synthesized using the same procedure as described in example 42 (Steps 1-3) substituting ethylamine for dimethylamine in Step 1. The crude reaction mixture was taken directly to the next step without purification.

### Step 2: Preparation of 4-{[(4,5-difluoro-2-{[(2,2,3,3-tetrafluoro-2,3-dihydro-1,4-benzodioxin-6-yl)amino]carbonyl}phenyl)amino]methyl}-N-ethylpyridine-2-carboxamide

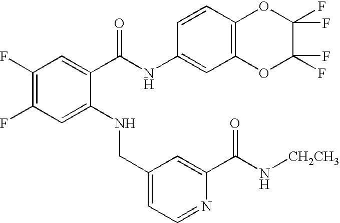

This compound was made using the procedure from example 42 except 2-amino-4,5-difluoro-N-(2,2,3,3-tetrafluoro-2,3-dihydro-1,4-benzodioxin-6-yl)benzamide was used instead of the 2-amino-N-(2,2-difluoro-1,3-benzodioxol-5-yl)-5-fluorobenzamide and {2-[(ethylamino)carbonyl]pyridin-4-yl}methyl methanesulfonate (from Step 1 above) was used in place of {2-[(dimethylamino)carbonyl]pyridin-4-yl}methyl methanesulfonate.

¹H NMR (DMSO-d₆) δ 10.42 (s, 1H), 8.78 (t, J=6 Hz, 1H), 8.54 (d, J=5 Hz, 2H), 8.13 (t, J=6 Hz, 1H), 7.98 (s, 1H), 7.89-7.81 (m, 3H), 7.59-7.46 (m, 3H), 6.59 (dd, J=6, 7 Hz, 1H), 4.57 (d, J=8 Hz, 2H), 3.30-3.25 (m, 2H), 1.07 (t, J=8 Hz, 3H); LCMS: 541 [M+H]⁺, RT 3.95 mm.

### Example 44

### N-ethyl-4-{[(2-{[(2,2,3,3-tetrafluoro-2,3-dihydro-1,4-benzodioxin-6-yl)amino]carbonyl}phenyl)amino]methyl}pyridine-2-carboxamide

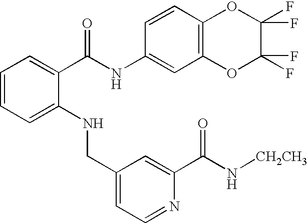

This compound was synthesized using the same synthetic route as Example 22 except {2-[(ethylamino)carbonyl]pyridin-4-yl}methyl methanesulfonate (Example 43, Step 1) was used in place of Intermediate B in Step 3.

¹H NMR (DMSO-d₆) δ 10.44 (s, 1H) 8.77 (t, J=8 Hz, 1H), 8.52 (d, J=8 Hz, 1H), 7.97-7.92, (m, 3H), 7.67 (d, J=8 Hz, 1H), 7.61 (d, J=10 Hz, 1H), 7.51-7.44 (m, 2H), 7.21 (t, J=8 Hz, 1H), 6.66 (t, J=7 Hz, 1H), 6.50 (d, J=9 Hz, 1H), 4.56 (d, J=8 Hz, 2H), 3.30-3.25 (m, 2H), 1.07 (t, J=8 Hz, 3H); LCMS: 505 [M+H]⁺, RT 3.81 min.

### Example 45

### 4-{[(2-{[(2,2-difluoro-1,3-benzodioxol-5-yl)amino]carbonyl}phenyl)amino]methyl}-N-ethylpyridine-2-carboxamide

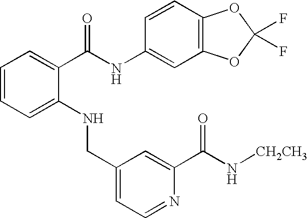

This compound was synthesized using the same synthetic route as Example 22 except {2-[(ethylamino)carbonyl]pyridin-4-yl}methyl methanesulfonate (Example 43, Step 1) was used in place of {2-[(dimethylamino)carbonyl]pyridin-4-yl}methyl methanesulfonate in step 3 and 2,2-difluoro-1,3-benzodioxol-5-amine was used in place of 2,2,3,3-tetrafluoro-2,3-dihydro-1,4-benzodioxin-6-amine in step 1.

¹H NMR (DMSO-d₆) δ 10.34 (s, 1H), 8.77 (t, J=8 Hz, 1H), 8.52 (d, J=6 Hz, 1H), 7.98-7.93, (m, 2H), 7.85 (s, 1H), 7.66 (d, J=10 Hz, 1H), 7.52-7.37 (m, 3H), 7.21 (t, J=8 Hz, 1H), 6.63 (t, J=7 Hz, 1H), 6.49 (d, J=9 Hz, 1H), 4.56 (d, J=6 Hz, 2H), 3.30-3.25 (m, 2H), 1.07 (t, J=8 Hz, 3H); LCMS: 455 [M+H]⁺, RT 3.53 min.

### Example 46

### 4-{[(2-{[(2,2-difluoro-1,3-benzodioxol-5-yl)amino]carbonyl}-4-fluorophenyl)amino]methyl}-N-ethylpyridine-2-carboxamide

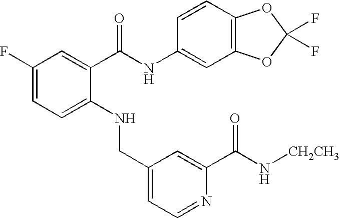

This compound was synthesized using the same synthetic route as Example 42 except in Step 3, {2-[(ethylamino)carbonyl]pyridin-4-yl}methyl methanesulfonate (Example 43, Step 1) was used in place of {2-[(dimethylamino)carbonyl]pyridin-4-yl}methyl methanesulfonate and the anthranilamide starting material is from Example 30.

¹H NMR (DMSO-d₆) δ 10.37 (s, 1H) 8.77 (t, J=8 Hz, 1H), 8.53 (d, J=4 Hz, 1H), 7.96 (s, 1H), 7.85-7.80, (m, 2H), 7.56 (dd, J=4, 10 Hz, 1H), 7.50 (d, J=5 Hz, 1H), 7.44 (dd, J=3, 11 Hz, 1H), 7.39 (d, J=9 Hz, 1H), 7.12 (td, J=3, 10 Hz, 1H), 6.48 (dd, J=4, 9 Hz, 1H), 4.55 (d, J=7 Hz, 2H), 3.30-3.25 (m, 2H), 1.07 (t, J=8 Hz, 3H); LCMS: 473 [M+H]⁺, RT 3.61 min.

### Example 47

### 4-{[(2-{[(2,2-difluoro-1,3-benzodioxol-5-yl)amino]carbonyl}-5-fluorophenyl)amino]methyl}-N-ethylpyridine-2-carboxamide

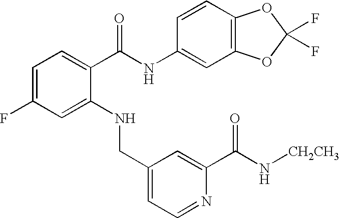

This compound was synthesized using the same synthetic route as Example 22 except in {2-[(ethylamino)carbonyl]pyridin-4-yl}methyl methanesulfonate (Example 43, Step 1) was used in place of {2-[(dimethylamino)carbonyl]pyridin-4-yl}methyl mathane sulfonate and 2-amino-N-(2,2-difluoro-1,3-benzodioxol-5-yl)-4-fluorobenzamide was used in place of 2-amino-N-(2,2,3,3-tetrafluoro-2,3-dihydro-1,4-benzodioxin-6-yl)benzamide in step 3.

¹H NMR (DMSO-d₆) δ 10.33 (s, 1H) 8.79 (t, J=8 Hz, 1H), 8.55 (d, J=4 Hz, 1H), 8.30 (t, J=6 Hz, 1H), 7.96 (s, 1H), 7.83 (s, 1H), 7.76 (t, J=8 Hz, 1H), 7.50 (d, J=5 Hz, 1H), 7.45-7.36 (m, 3H), 6.46 (td, J=2, 8 Hz, 1H), 6.32 (dd, J=2, 9 Hz, 1H), 4.58 (d, J=7 Hz, 2H), 3.30-3.23 (m, 2H), 1.07 (t, J=8 Hz, 3H); LCMS: 473 [M+H]⁺, RT 3.61 min.

### Example 48

### N-(2,2-difluoro-1,3-benzodioxol-5-yl)-2-({[2-(dimethylamino)pyrimidin-4-yl]methyl}amino)benzamide

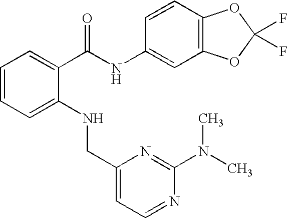

This compound was prepared using the same procedure as in Example 5 except in Step 1, 2-(dimethylamino)pyrimidine-4-carbaldehyde (Reference for preparation is in Table 1) was used in place of 4-pyridinecarboxaldehyde.

¹H NMR (CD₂Cl₂-d₂) δ 8.31 (s, 1H), 8.20 (d, J=2.7 Hz, 1H), 7.77 (s, 1H), 7.53 (d, J=8.0 Hz, 1H), 7.34 (t, J=8.0 Hz, 1H), 7.07 (s, 2H), 6.70 (s, 2H), 6.47 (d, J=4.0 Hz, 1H), 4.32 (d, J=4.0 Hz, 2H), 3.23 (s, 6H); LCMS: 428 [M+H]⁺, RT 2.92 min.

### Example 49

### N-(2,2-difluoro-1,3-benzodioxol-5-yl)-2-({[2-(methylamino)pyrimidin-4-yl]methyl}amino)benzamide

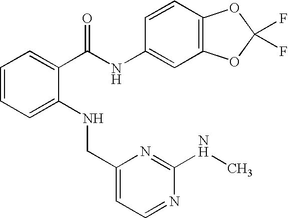

This compound was synthesized using the same synthetic route as Example 5 except 2-(methylamino)pyrimidine-4-carbaldehyde (Reference for preparation is in Table 1) was used in place of 4-pyridinecarboxaldehyde.

¹H NMR (DMSO-d₆) δ 10.3 (s, 1H), 8.15 (s, 1H), 7.87 (s, 1H), 7.66 (d, J=8.0 Hz, 1H), 7.46-7.36 (m, 3H), 7.05 (s, 1H), 6.66-6.55 (m, 2H), 6.48 (d, J=8.0 Hz, 1H), 4.27 (d, J=8.0 Hz, 2H), 2.81 (s, 3H); LCMS: 414 [M+H]⁺, RT 3.13 min.

### Example 50

### 2-({[2-(dimethylamino)pyrimidin-4-yl]methyl}amino)-N-(2,2,3,3-tetrafluoro-2,3-dihydro-1,4-benzodioxin-6-yl)benzamide

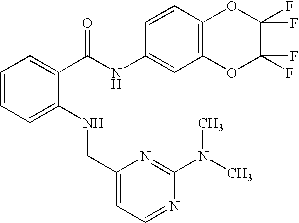

This compound was synthesized using the same synthetic route as Example 5 except 2,2,3,3-tetrafluoro-2,3-dihydro-1,4-benzodioxin-6-amine is used in place 2,2-difluoro-1,3-benzodioxol-5-amine in step 2 and 2-(methylamino)pyrimidine-4-carbaldehyde (Reference for preparation is in Table 1) was used in place of 4-pyridinecarboxaldehyde in step 1.

¹H NMR (DMSO-d₆) δ 10.4 (s, 1H), 8.22 (d, J=8.0 Hz, 2H), 7.92 (d, J=4.0 Hz, 1H), 7.66-7.58 (m, 2H), 7.44 (d, J=8.0 Hz, 1H), 7.31 (t, J=4.0 Hz, 1H), 6.68-6.62 (m, 2H), 6.51 (d, J=4.0 Hz, 1H), 4.30 (d, J=4.0 Hz, 2H), 3.15 (s, 6H); LCMS: 478 [M+H]⁺, RT 3.25 min.

### Example 51

### 2-({[2-(methylamino)pyrimidin-4-yl]methyl}amino)-N-(2,2,3,3-tetrafluoro-2,3-dihydro-1,4-benzodioxin-6-yl)benzamide

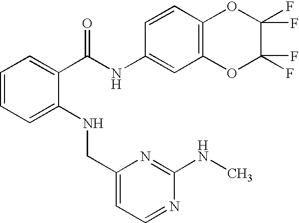

This compound was synthesized using the same synthetic route as Example 5 except 2-(methylamino)pyrimidine-4-carbaldehyde (Reference for preparation is in Table 1) was used in place of 4-pyridinecarboxaldehyde in step 1, and in Step 2,2,2,3,3-tetrafluoro-2,3-dihydro-1,4-benzodioxin-6-amine was used in place 2,2-difluoro-1,3-benzodioxol-5-amine.

¹H NMR (DMSO-d₆) δ 8.12 (d, J=8.0 Hz, 1H), 7.81 (s, 1H), 7.66 (d, J=8.0 Hz, 1H), 7.49-7.47 (m, 1H), 7.32-7.22 (m, 2H), 6.72-6.66 (m, 3H), 4.34 (s, 2H), 2.96 (s, 3H); LCMS: 464 [M+H]⁺, RT 3.38 min.

### Example 52

### 4-{[(2-{[(2,2-difluoro-1,3-benzodioxol-5-yl)amino]carbonyl}-phenyl)amino]methyl}-N-(2-methoxyethyl)pyridine-2-carboxamide

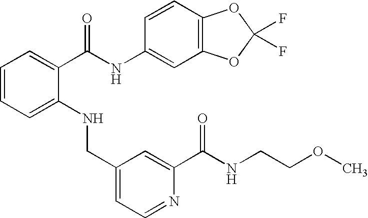

### Step 1: Preparation of (2-{[(2-methoxyethyl)amino]carbonyl}pyridin-4-yl)methyl methanesulfonate

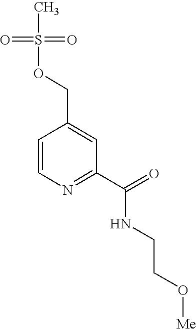

The title compound was synthesized using the same procedure as described in example 42 Steps 1-3 substituting 2-methoxyethylamine for dimethylamine. The crude reaction mixture was taken directly to the next step without purification.

### Step 2: Preparation of 4-{[(2-{[(2,2-difluoro-1,3-benzodioxol-5-yl)amino]-carbonyl}phenyl)amino]methyl}-N-(2-methoxyethyl)pyridine-2-carboxamide

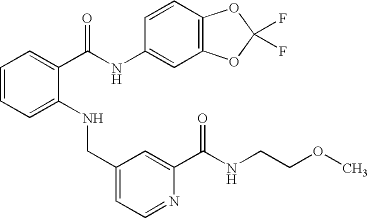

This compound was made using the procedure from example 22 step 3 except (2-{[(2-methoxyethyl)amino]carbonyl}pyridin-4-yl)methylmethanesulfonate from step 1 was used instead of intermediate B and 2-amino-N-(2,2-difluoro-1,3-benzodioxol-5-yl)benzamide instead of 2-amino-N-(2,2,3,3-tetrafluoro-2,3-dihydro-1,4-benzodioxin-6-yl)benzamide in step 3.

¹H NMR (CD₂Cl₂) δ8.39 (d, 1H, J=5.0 Hz), 8.16 (s, 2H), 8.03 (s, 1H), 8.00, (s, 1H), 7.62 (s, 1H), 7.46 (d, 1H, 9 Hz), 7.34 (d, 1H, 6 Hz), 7.15-7.04 (m, 2H), 6.96 (d, 1H, 8 Hz), 6.54 (t, 1H, 8 Hz), 7.34 (d, 1H, 6 Hz), 4.42 (s, 2H), 3.53-3.42 (m, 4H), 3.27 (s, 3H); MS [M+H]⁺=485; LC-MS RT=3.25 min.

### Example 53

### 4-{[(2-{[(2,2-difluoro-1,3-benzodioxol-5-yl)amino]carbonyl}-4-fluoro-phenyl)amino]methyl}-N-(2-methoxyethyl)pyridine-2-carboxamide

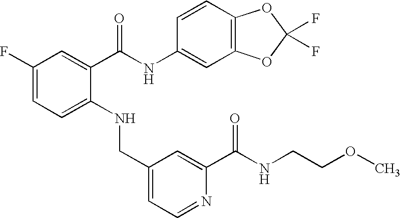

This compound was made using the procedure from example 22 step 3 except (2-{[(2-methoxyethyl)amino]carbonyl}pyridin-4-yl)methylmethanesulfonate from step 1 of example 52 was used instead of intermediate B and 2-amino-N-(2,2-difluoro-1,3-benzodioxol-5-yl)-5-fluorobenzamide was used instead of 2-amino-N-(2,2,3,3-tetrafluoro-2,3-dihydro-1,4-benzodioxin-6-yl)benzamide in step 3.

¹H NMR (CD₂Cl₂) δ8.41 (d, 1H, J=5.0 Hz), 8.19 (s, 2H), 8.02 (s, 1H), 7.78, (bs, 1H), 7.60 (s, 1H), 7.34 (d, 1H, 5 Hz), 7.20 (d, 1H, 10 Hz), 7.06 (d, 1H, 8 Hz), 6.96 (d, 1H, 8 Hz), 6.87 (m, 1H), 6.33 (q, 1H, 5 Hz), 4.42 (s, 2H), 3.53-3.42 (m, 4H), 3.27 (s, 3H); MS [M+H]⁺=503; LC-MS RT=3.39 min.

### Example 54

### N-(2,2,3,3,7-pentafluoro-2,3-dihydro-1,4-benzodioxin-6-yl)-2-[(pyridin-4-ylmethyl)amino]benzamide

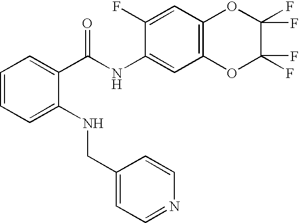

The compound can be synthesized using the same synthetic route as in Example 5 except in Step 2,2,2,3,3,7-pentafluoro-2,3-dihydro-1,4-benzodioxin-6-amine is used in place of 2,2-difluoro-1,3-benzodioxol-5-amine.

### Example 55

### N-(3-chloro-2,2,3-trifluoro-2,3-dihydro-1,4-benzodioxin-6-yl)-2-[(pyridin-4-ylmethyl)amino]benzamide

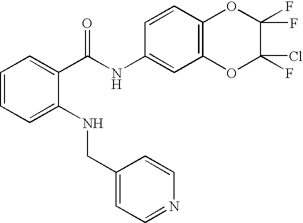

The compound can be synthesized using the same synthetic route as in Example 5 except in Step 2,3-chloro-2,2,3-trifluoro-2,3-dihydro-1,4-benzodioxin-6-amine is used in place of 2,2-difluoro-1,3-benzodioxol-5-amine.

### Example 56

### 2-[(pyridin-4-ylmethyl)amino]-N-(2,3,3,7-tetrafluoro-2,3-dihydro-1,4-benzodioxin-6-yl)benzamide

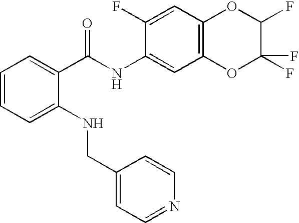

The compound can be synthesized using the same synthetic route as in Example 5 except in Step 2,2,3,3,7-tetrafluoro-2,3-dihydro-1,4-benzodioxin-6-amine is used in place of 2,2-difluoro-1,3-benzodioxol-5-amine.

### Example 57

### 2-[(pyridin-4-ylmethyl)amino]-N-(2,2,3-trichloro-3-fluoro-2,3-dihydro-1,4-benzodioxin-6-yl)benzamide

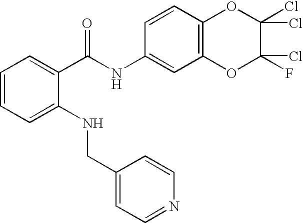

The compound can be synthesized using the same synthetic route as in Example 5 except in Step 2,2,2,3-trichloro-3-fluoro-2,3-dihydro-1,4-benzodioxin-6-amine is used in place of 2,2-difluoro-1,3-benzodioxol-5-amine.

### Example 58

### 2-[(pyridin-4-ylmethyl)amino]-N-(2,2,3-trifluoro-7-methyl-2,3-dihydro-1,4-benzodioxin-6-yl)benzamide

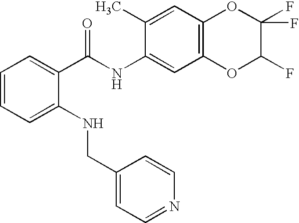

The compound can be synthesized using the same synthetic route as in Example 5 except in Step 2,2,2,3-trifluoro-7-methyl-2,3-dihydro-1,4-benzodioxin-6-amine is used in place of 2,2-difluoro-1,3-benzodioxol-5-amine.

### Example 59

### N-(7-chloro-2,2,4,4-tetrafluoro-4H-1,3-benzodioxin-6-yl)-2-[(pyridin-4-ylmethyl)amino]benzamide

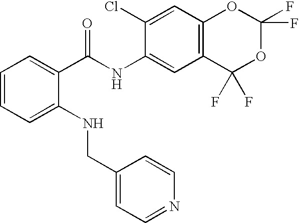

The compound can be synthesized using the same synthetic route as in Example 5 except in Step 2,7-chloro-2,2,4,4-tetrafluoro-4H-1,3-benzodioxin-6-amine is used in place of 2,2-difluoro-1,3-benzodioxol-5-amine.

### Example 60

### 2-[(pyridin-4-ylmethyl)amino]-N-(2,2,3-trifluoro-2,3-dihydro-1,4-benzodioxin-6-yl)benzamide

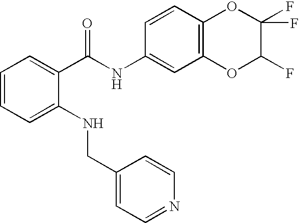

The compound can be synthesized using the same synthetic route as in Example 5 except in Step 2,2,2,3-trifluoro-2,3-dihydro-1,4-benzodioxin-6-amine is used in place of 2,2-difluoro-1,3-benzodioxol-5-amine.

### Example 61

### N-(6-chloro-2,2-difluoro-1,3-benzodioxol-5-yl)-2-[(pyridin-4-ylmethyl)amino]benzamide

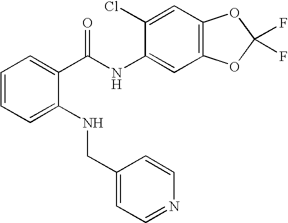

The compound can be synthesized using the same synthetic route as in Example 5 except in Step 2,6-chloro-2,2-difluoro-1,3-benzodioxol-5-amine is used in place of 2,2-difluoro-1,3-benzodioxol-5-amine.

### Example 62

### N-(2,2-difluoro-4-methyl-1,3-benzodioxol-5-yl)-2-[(pyridin-4-ylmethyl)amino]benzamide

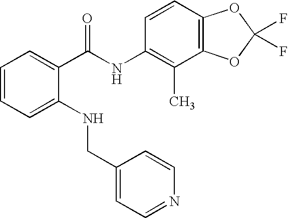

The compound can be synthesized using the same synthetic route as in Example 5 except in Step 2,2,2-difluoro-4-methyl-1,3-benzodioxol-5-amine is used in place of 2,2-difluoro-1,3-benzodioxol-5-amine.

### Example 63

### N-methyl-4-{[(2-{[(2,2,3,3,7-pentafluoro-2,3-dihydro-1,4-benzodioxin-6-yl)amino]carbonyl}phenyl)amino]methyl}pyridine-2-carboxamide

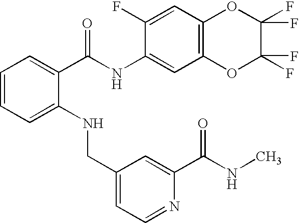

This compound is synthesized using the same synthetic route as in Example 3 except in Step 1,2,2,3,3,7-pentafluoro-2,3-dihydro-1,4-benzodioxin-6-amine is used in place of 2,2,3,3-tetrafluoro-2,3-dihydro-1,4-benzodioxin-6-amine.

### Example 64

### 4-{[(2-{[(3-chloro-2,2,3-trifluoro-2,3-dihydro-1,4-benzodioxin-6-yl)amino]carbonyl}phenyl)amino]methyl}-N-methylpyridine-2-carboxamide

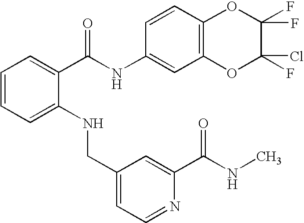

This compound is synthesized using the same synthetic route as in Example 3 except in Step 1,3-chloro-2,2,3-trifluoro-2,3-dihydro-1,4-benzodioxin-6-amine is used in place of 2,2,3,3-tetrafluoro-2,3-dihydro-1,4-benzodioxin-6-amine.

### Example 65

### N-methyl-4-{[(2-{[(2,3,3,7-tetrafluoro-2,3-dihydro-1,4-benzodioxin-6-yl)amino]carbonyl}phenyl)amino]methyl}pyridine-2-carboxamide

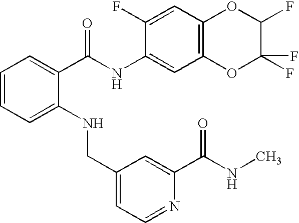

This compound is synthesized using the same synthetic route as in Example 3 except in Step 1,2,3,3,7-tetrafluoro-2,3-dihydro-1,4-benzodioxin-6-amine is used in place of 2,2,3,3-tetrafluoro-2,3-dihydro-1,4-benzodioxin-6-amine.

### Example 66

### N-methyl-4-{[(2-{[(2,2,3-trichloro-3-fluoro-2,3-dihydro-1,4-benzodioxin-6-yl)amino]carbonyl}phenyl)amino]methyl}pyridine-2-carboxamide

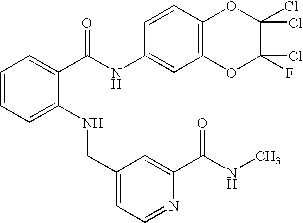

This compound is synthesized using the same synthetic route as in Example 3 except in Step 1,2,2,3-trichloro-3-fluoro-2,3-dihydro-1,4-benzodioxin-6-amine is used in place of 2,2,3,3-tetrafluoro-2,3-dihydro-1,4-benzodioxin-6-amine.

### Example 67

### N-methyl-4-{[(2-{[(2,2,3-trifluoro-7-methyl-2,3-dihydro-1,4-benzodioxin-6-yl)amino]carbonyl}phenyl)amino]methyl}pyridine-2-carboxamide

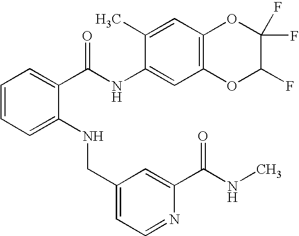

This compound is synthesized using the same synthetic route as in Example 3 except in Step 1,2,2,3-trifluoro-7-methyl-2,3-dihydro-1,4-benzodioxin-6-amine is used in place of 2,2,3,3-tetrafluoro-2,3-dihydro-1,4-benzodioxin-6-amine.

### Example 68

### 4-{[(2-{[(7-chloro-2,2,4,4-tetrafluoro-4H-1,3-benzodioxin-6-yl)amino]carbonyl}phenyl)amino]methyl}-N-methylpyridine-2-carboxamide

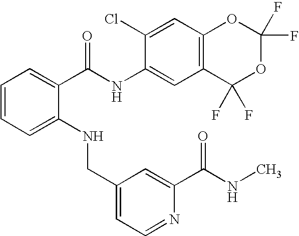

This compound is synthesized using the same synthetic route as in Example 3 except in Step 1,7-chloro-2,2,4,4-tetrafluoro-4H-1,3-benzodioxin-6-amine is used in place of 2,2,3,3-tetrafluoro-2,3-dihydro-1,4-benzodioxin-6-amine.

### Example 69

### N-methyl-4-{[(2-{[(2,2,3-trifluoro-2,3-dihydro-1,4-benzodioxin-6-yl)amino]carbonyl}phenyl)amino]methyl}pyridine-2-carboxamide

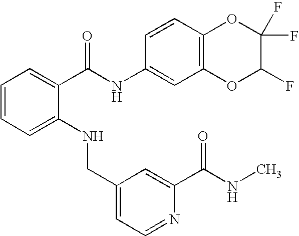

This compound is synthesized using the same synthetic route as in Example 3 except in Step 1,2,2,3-trifluoro-2,3-dihydro-1,4-benzodioxin-6-amine is used in place of 2,2,3,3-tetrafluoro-2,3-dihydro-1,4-benzodioxin-6-amine.

### Example 70

### 4-{[(2-{[(6-chloro-2,2-difluoro-1,3-benzodioxol-5-yl)amino]carbonyl}phenyl)amino]methyl}-N-methylpyridine-2-carboxamide

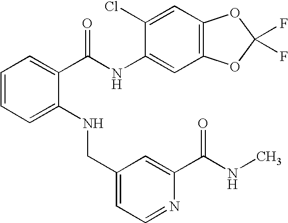

This compound is synthesized using the same synthetic route as in Example 3 except in Step 1,6-chloro-2,2-difluoro-1,3-benzodioxol-5-amine is used in place of 2,2,3,3-tetrafluoro-2,3-dihydro-1,4-benzodioxin-6-amine.

### Example 71

### 4-{[(2-{[(2,2-difluoro-4-methyl-1,3-benzodioxol-5-yl)amino]carbonyl}phenyl)amino]methyl}-N-methylpyridine-2-carboxamide

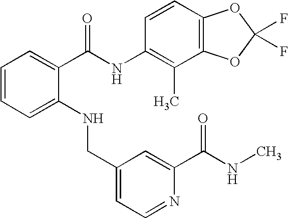

This compound is synthesized using the same synthetic route as in Example 3 except in Step 1,2,2-difluoro-4-methyl-1,3-benzodioxol-5-amine is used in place of 2,2,3,3-tetrafluoro-2,3-dihydro-1,4-benzodioxin-6-amine.

### Example 72

### 2-({[2-(methylamino)pyrimidin-4-yl]methyl}amino)-N-(2,2,6-trifluoro-1,3-benzodioxol-5-yl)benzamide

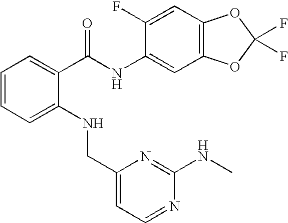

This compound can be synthesized using the same synthetic route as in Example 5 except in Step 1, 2-(methylamino)pyrimidine-4-carbaldehyde is used in place of 2-(dimethylamino)pyrimidine-4-carbaldehyde, and in Step 2,2,2,6-trifluoro-1,3-benzodioxol-5-amine is used in place of 2,2-difluoro-1,3-benzodioxol-5-amine.

### Example 73

### N-(3,3-difluoro-2,3-dihydro-1,4-benzodioxin-6-yl)-2-({[2 (methylamino)pyrimidin-4-yl]methyl}amino)benzamide

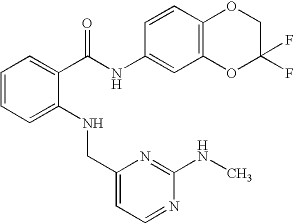

This compound can be synthesized using the same synthetic route as in Example 5 except in Step 1, 2-(methylamino)pyrimidine-4-carbaldehyde is used in place of 2-(dimethylamino)pyrimidine-4-carbaldehyde, and in Step 2, 3,3-difluoro-2,3-dihydro-1,4-benzodioxin-6-amine is used in place of 2,2-difluoro-1,3-benzodioxol-5-amine.

### Example 74

### N-cyclopropyl-4-{[(2-{[(2,2-difluoro-1,3-benzodioxol-5-yl)amino]carbonyl}phenyl)amino]methyl}pyridine-2-carboxamide

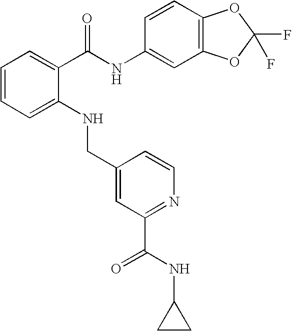

### Step 1: Preparation of methyl 2-[(cyclopropylamino)carbonyl]isonicotinate

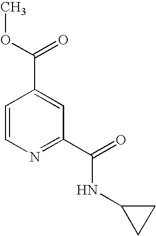

Pyridine-2,4-dicarboxylic acid dimethyl ester (1.0 g, 0.051 mol) was taken up in DCM (9.0 ml) and stirred at room temperature until it all dissolved. The solution was cooled to 0° C. and MgCl₂ (312 mg, 3.27 mmol) was added and allowed to stir for 30 minutes. Cyclopropylamine (438 mg, 7.68 mmol as 2M solution in DCM) was added dropwise over the course of 3 hrs. The solution was stirred for 12 hours. The crude reaction mixture was quenched with water (50 ml) and pH 4 buffer (50 ml) was added to neutralize the solution. The aqueous layer was extracted with DCM (3×150 ml). The organic fractions were combined, dried with sodium sulfate and concentrated under vacuum. The white residue was determined to be the product in about 90% purity. It was taken directly to the next step without further purification. LCMS: 221.1 [M+H]+, RT 1.97 min.

### Step 2: Preparation of N-cyclopropyl-4-(hydroxymethyl)pyridine-2-carboxamide

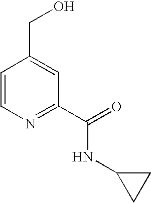

Methyl 2-[(cyclopropylamino)carbonyl]isonicotinate (11.20 g, 0.051 mol) was dissolved in MeOH (30 ml) and allowed to stir for 15 minutes. The solution was cooled to 0° C. and NaBH₄ (384 mg, 10.17 mmol) was added in portions over the course of 1 hr. Additional NaBH₄ (576 mg, 15.25 mmol) was added over the course of 3 hrs. The solution was allowed to stir for 12 hours at rt. The crude mixture was directly added to a silica plug without working it up and eluted with MeOH (200 ml). The eluant was concentrated and allowed to dry under vacuum for 3 hours. A white residue (850 mg, 86.95%) was recovered and determined to be N-cyclopropyl-4-(hydroxymethyl)pyridine-2-carboxamide. The crude product was taken to the next step. LCMS: 193.0 [M+H]+, RT 1.19 min.

### Step 3: Preparation of {2-[(cyclopropylamino)carbonyl]pyridin-4-yl}methyl methanesulfonate

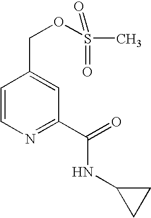

To a solution of N-cyclopropyl-4-(hydroxymethyl)pyridine-2-carboxamide (9.78 g, 58.9 mmol) in THF (250 mL) was added triethyl amine (12.3 mL, 88.3 mmol). The reaction was cooled to 0° C. and methanesulfonyl chloride (5.5 mL, 70.6 mmol) was added dropwise over 15 min. The reaction was allowed to slowly come to room temperature with stirring for 3 h. The resulting solution was concentrated, re-dissolved in EtOAc (200 mL), transferred into a separatory funnel and the organic layer was washed with cold satd. NaHCO₃ (2×200 mL). The organic layer was dried (MgSO₄), filtered and concentrated to afford 1.16 g of the above crude compound as a solid which was taken to the next step without further purification. (4.75 mmol, yield 81%). LCMS: 271 [M+H]+, RT 1.24 min.

### Step 4: Preparation of N-cyclopropyl-4-{[(2-{[(2,2-difluoro-1,3-benzodioxol-5-yl)amino]carbonyl}phenyl)amino]methyl}pyridine-2-carboxamide

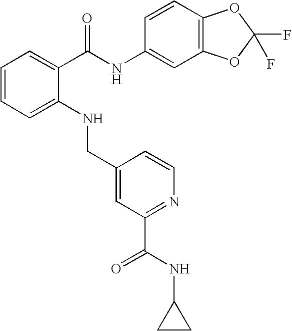

{2-[(Cyclopropylamino)carbonyl]pyridin-4-yl}methyl methanesulfonate (60.0 mg, 0.22 mmol) was dissolved in DMF (1 ml). To the solution was added NaI (49.9 mg, 0.33 mmol) and the mixture was allowed to stir for 10 minutes at room temperature. A solution of 2-Amino-N-(2,2-difluoro-benzo[1,3]dioxol-5-yl)-benzamide (Intermediate W) (194.6 mg, 0.67 mmol) in DMF (1 ml) was added via syringe to the reaction mixture and allowed to stir for 12 hrs at 60° C. The reaction mixture was taken up in EtOAc (50 ml) and washed sequentially with water (2×50 ml) followed by brine (50 ml). The organic fraction was dried with sodium sulfate and concentrated to half its volume in vacuo. The remaining liquid was allowed to sit for 12 hrs until a white solid had precipitated out. The white solid was collected by filtration and 17.6 mg (17%) of the title compound was recovered. ¹HNMR (DMSO-d6) δ 10.36 (s, 1H) 8.69 (d, 1H, J=4.8 Hz), 8.51 (d, 1H, J=5.0 Hz), 7.96, (s, 2H), 7.86 (s, 1H), 7.67 (d, 1H, J=7.5 Hz), 7.51 (d, 1H, J=7.5 Hz), 7.46 (d, 1H, J=9.8), 7.38 (d, 1H, J=8.7 Hz), 7.22 (t, 1H, J=7.3 Hz), 6.64 (t, 1H, J=7.3 Hz), 6.49 (d, 1H, J=7.5 Hz), 4.57 (d, 2H, J=5.1 Hz), 2.85 (m, 1H), 1.22 (s, 2H), 0.65 (s, 2H); LCMS: 467 [M+H]+, RT 3.85 min.

### Example 75

### 4-{[(2-{[(2,2-Difluoro-1,3-benzodioxol-5-yl)amino]carbonyl}phenyl)amino]methyl}pyridine-2-carboxamide

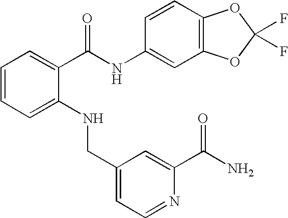

A solution of 2-amino-N-(2,2-difluoro-1,3-benzodioxol-5-yl)benzamide (1.50 g, 5.13 mmol) (Intermediate W) and [2-(aminocarbonyl)pyridin-4-yl]methyl methanesulfonate (Intermediate G) (1.18 g, 5.13 mmol) in anhydrous DMF (10 ml) was stirred under nitrogen as sodium iodide (769 mg, 5.13 mmol) was added. The reaction flask was wrapped in foil to exclude light and then heated with stirring under nitrogen at 60° C. for 2.25 hr. The resultant dark solution was diluted into a mixture of 50% saturated brine (100 ml) and ethyl acetate (250 ml). After shaking, the phases were separated and the organic product extract was washed twice with water and then with brine. It was then dried (Na₂SO₄) and concentrated under reduced pressure to give the title compound as a dark oil which was then dissolved in dichloromethane (30 ml). After a few minutes, crystals formed which were removed by filtration and washed with dichloromethane to yield semipure (ca. 87% pure) product. The combined filtrate and wash were chromatographed on silica gel using a gradient from 30-100% ethyl acetate in hexane to give additional semipure (ca. 80% pure) material. Both portions of semipure material was dissolved as much as possible in hot dichloromethane (50 ml) and the resultant slurry was cooled in a refrigerator overnight and then the pure title compound was collected by filtration, washed with dichloromethane and dried in vacuo to give pure 4-{[(2-{[(2,2-difluoro-1,3-benzodioxol-5-yl)amino]carbonyl}phenyl)amino]-methyl}pyridine-2-carboxamide (1.10 g, 50%, 95% pure): ¹H NMR (300 MHz, DMSO-d6) δ 10.36 (s, 1H), 8.53 (d, 1H), 8.08 (s, 1H), 7.95 (m, 2H), 7.88 (s, 1H), 7.68 (d, 1H), 7.62 (s, 1H), 7.50 (d, 1H), 7.46 (d, 1H), 7.39 (d, 1H), 7.23 (t, 1H), 6.66 (t, 1H), 6.51 (d, 1H), and 4.59 ppm (d, 2H); ES-MS m/z 427.0 [M+H]+ and 449.1 [M+Na]+, HPLC RT (min) 3.18.

### Example 76

### Methyl 4-{[(2-{[(2,2-difluoro-1,3-benzodioxol-5-yl)amino]carbonyl}phenyl)amino]methyl}pyridine-2-carboxylate

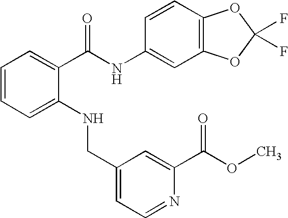

A slurry of 4-{[(2-{[(2,2-difluoro-1,3-benzodioxol-5-yl)amino]carbonyl}phenyl)amino]methyl}pyridine-2-carboxamide (3.39 g, 7.95 mmol) and 1,1-dimethoxy-N,N-dimethylmethanamine (3.38 ml, 25.4 mmol) in methanol (33.9 ml) was stirred under nitrogen in a sealed glass pressure reactor (150 ml) fitted with a SS pressure gauge at 50° C. for 2.5 hr. All materials had dissolved after 35 min heating and only a slight pressure (5 psi) was observed as the reaction proceeded. The reaction was cooled, opened and the contents were then evaporated in vacuo. The residue was dissolved in dichloromethane and purified by MPLC (Isco®, Flash 120 g cartridge, gradient from 0-100% EtOAc/hexane) to afford Methyl 4-{[(2-{[(2,2-difluoro-1,3-benzodioxol-5-yl)amino]carbonyl}phenyl)amino]methyl}pyridine-2-carboxylate as a white solid (2.08 g, 59%): ¹H NMR (300 MHz, DMSO-d6) δ 10.33 (s, 1H), 8.60 (d, 1H), 8.01 (s, 1H), 7.93 (t, 1H), 7.87 (s, 1H), 7.67 (d, 1H), 7.56 (d, 1H), 7.44 (d, 1H), 7.39 (d, 1H), 7.22 (t, 1H), 6.66 (t, 1H), 6.51 (d, 1H), 4.58 (d, 2H), and 4.85 ppm (s, 3H); ES-MS m/z 442.1 [M+H]+ and 464.1 [M+Na]+, HPLC RT (min) 3.23.

### Example 77

### 4-{[(2-{[(2,2-Difluoro-1,3-benzodioxol-5-yl)amino]carbonyl}phenyl)amino]methyl}-N-(2-furylmethyl)pyridine-2-carboxamide

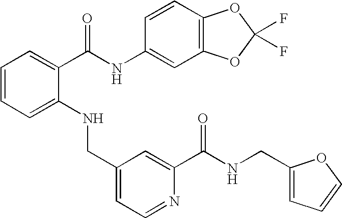

A slurry of 4-{[(2-{[(2,2-difluoro-1,3-benzodioxol-5-yl)amino]carbonyl}phenyl)amino]methyl}pyridine-2-carboxamide (50 mg, 0.12 mmol) and 1,1-dimethoxy-N,N-dimethylmethanamine (0.05 ml, 0.35 mmol) in methanol (0.5 ml) was stirred under nitrogen in a septum sealed vial at 50° C. for 2.5 hr to convert all of the starting material to Methyl 4-{[(2-{[(2,2-difluoro-1,3-benzodioxol-5-yl)amino]carbonyl}phenyl)amino]methyl}pyridine-2-carboxylate. A portion of 1-(2-furyl)methanamine (0.09 ml, (1.06 mmol) was injected via microliter syringe and the resultant solution was heated at 50° C. for 7.5 hr and then at 65° C. for 1.75 hr. The resultant product solution was purified directly by HPLC on a YMC-Pack Pro 18® (150×20 mm I.D.) column using a gradient from 10-50% acetonitrile in water (plus 0.05% TFA). The best fractions were combined, partially evaporated, mixed with aqueous saturated sodium bicarbonate and extracted 3× with dichloromethane. Combined extracts were dried (Na₂SO₄) and concentrated under reduced pressure to give the title compound as a fine white solid (33 mg, 56%): ¹H NMR (300 MHz, DMSO-d6) δ 10.34 (s, 1H), 9.11 (t, 1H), 8.54 (d, 1H), 7.99 (s, 1H), 7.94 (t, 1H), 7.87 (s, 1H), 7.67 (d, 1H), 7.52 (d, 1H), 7.44 (d, 1H), 7.39 (d, 1H), 7.22 (t, 1H), 6.65 (t, 1H), 6.51 (d, 1H), 6.34 (d, 1H), 6.21 (d, 1H), 4.58 (d, 2H), and 4.44 ppm (d, 2H); ES-MS m/z 507.6 [M+H]+, HPLC RT (min) 3.79.

### Example 78

### 4-{[(2-{[(2,2-Difluoro-1,3-benzodioxol-5-yl)amino]carbonyl}phenyl)amino]methyl}-N-[(2,2-dimethyl-1,3-dioxolan-4-yl)methyl]pyridine-2-carboxamide

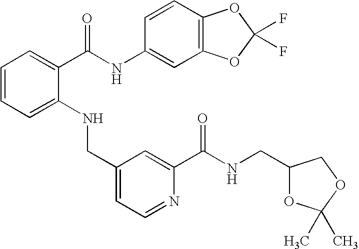

A solution of Methyl 4-{[(2-{[(2,2-difluoro-1,3-benzodioxol-5-yl)amino]carbonyl}phenyl)amino]methyl}pyridine-2-carboxylate (80 mg, 0.18 mmol) and 1-(2,2-dimethyl-1,3-dioxolan-4-yl)methanamine (0.05 ml, 0.36 mmol) in 1 ml methanol was heated in a sealed tube for 16 hr and the resultant product solution then purified by preparative HPLC as in the case of Example 77 to yield pure 4-{[(2-{[(2,2-difluoro-1,3-benzodioxol-5-yl)amino]carbonyl}phenyl)amino]methyl}-N-[(2,2-dimethyl-1,3-dioxolan-4-yl)methyl]pyridine-2-carboxamide (68 mg, 69%): ¹H NMR (300 MHz, CD₃OD) δ 8.52 (d, 1H), 8.10 (s, 1H), 7.76 (s, 1H), 7.67 (d, 1H), 7.54 (d, 1H), 7.33 (d, 1H), 7.25 (t, 1H), 7.16 (d, 1H), 6.70 (t, 1H), 6.54 (d, 1H), 4.60 (s, 2H), 4.31 (pent., 1H), 4.06 (dd, 1H), 3.71 (dd, 1H), 3.54 (d, 2H), 1.41 (s, 3H), and 1.32 ppm (s, 3H); ES-MS m/z 541.3 [M+H]+ and 563.3 [M+Na]+, HPLC RT (min) 3.27.

### Example 79

### 4-{[(2-{[(2,2-Difluoro-1,3-benzodioxol-5-yl)amino]carbonyl}phenyl)amino]methyl}-N-(2,3-dihydroxypropyl)pyridine-2-carboxamide

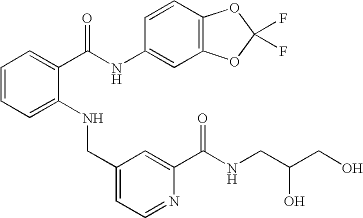

A solution of 4-{[(2-{[(2,2-difluoro-1,3-benzodioxol-5-yl)amino]carbonyl}-phenyl)amino]-methyl}-N-[(2,2-dimethyl-1,3-dioxolan-4-yl)methyl]pyridine-2-carboxamide (50 mg, 0.09 mmol) in 1 ml acetone was stirred with aqueous hydrochloric acid (2N, 1 ml) for 16 hr. The product solution was evaporated in vacuo and the resultant residue was purified by preparative HPLC as in the case of Example 77 to yield pure 4-{[(2-{[(2,2-difluoro-1,3-benzodioxol-5-yl)amino]carbonyl}phenyl)amino]-methyl}-N-(2,3-dihydroxypropyl)pyridine-2-carboxamide (45 mg, 97%): ¹H NMR (300 MHz, CD₃OD) δ 8.78 (d, 1H), 8.54 (s, 1H), 8.09 (d, 1H), 7.85 (s, 1H), 7.74 (d, 1H), 7.40 (d, 1H), 7.30 (t, 1H), 7.22 (d, 1H), 6.81 (t, 1H), 6.59 (d, 1H), 4.86 (s, 2H), 3.86 (pent., 1H), 3.64 (dd, 1H), 3.55 (d, 2H), and 3.46 ppm (dd, 1H); ES-MS m/z 501.1 [M+H]+ and 423.2 [M+Na]+, HPLC RT (min) 3.00.

### Example 80

### 4-{[(2-{[(2,2-Difluoro-1,3-benzodioxol-5-yl)amino]carbonyl}phenyl)amino]methyl}-N,N-dimethylpyridine-2-carboxamide

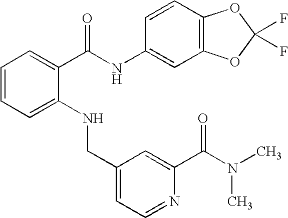

Careful chromatography of the side products from one preparation of Methyl 4-{[(2-{[(2,2-difluoro-1,3-benzodioxol-5-yl)amino]carbonyl}phenyl)amino]methyl}pyridine-2-carboxylate (Example 76) yielded a small amount (2.3% yield) of pure 4-{[(2-{[(2,2-difluoro-1,3-benzodioxol-5-yl)amino]carbonyl}phenyl)amino]methyl}-N,N-dimethylpyridine-2-carboxamide as a side product: ¹H NMR (300 MHz, CD₃OD) δ 8.52 (bs, 1H), 7.78 (s, 1H), 7.68 (d, 1H), 7.62 (bs, 1H), 7.58 (d, 1H), 7.33 (d, 1H), 7.23 (t, 1H), 7.16 (d, 1H), 6.70 (t, 1H), 6.54 (d, 1H), 4.60 (s, 2H), 3.08 (s, 3H) and 2.93 ppm (s, 3H); ES-MS m/z 455.2 [M+H]+ and 477.2 [M+Na]+, HPLC RT (min) 3.47.

### Example 81

### N-(tert-butyl)-4-{[(2-{[(2,2-difluoro-1,3-benzodioxol-5-yl)amino]carbonyl}phenyl)amino]methyl}pyridine-2-carboxamide

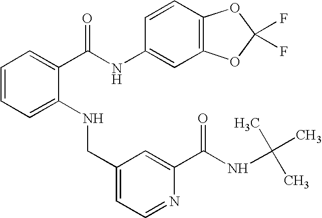

A slurry of 2-{[(2-cyanopyridin-4-yl)methyl]amino}-N-(2,2-difluoro-1,3-benzodioxol-5-yl)benzamide (200 mg, 0.49 mmol) in 2-methylpropan-2-ol (2 ml) was stirred under nitrogen at 70° C. as sulfuric acid (conc., 0.5 ml) was added dropwise. After stirring another 45 min at this temperature, the reaction mixture was diluted with water and ethyl acetate was added followed by aqueous potassium carbonate (10%) added dropwise to adjust the water phase to pH 10. The organic phase was dried (Na₂SO₄) and concentrated under reduced pressure to give a residue of crude product which was chromatographed on silica gel using a gradient from 5-10% EtOAc in hexane elution to give pure N-(tert-butyl)-4-{[(2-{[(2,2-difluoro-1,3-benzodioxol-5-yl)amino]carbonyl}phenyl)amino]methyl}pyridine-2-carboxamide (230 mg, 97%): ¹H NMR (300 MHz, DMSO-d6) δ 10.39 (s, 1H), 8.51 (d, 1H), 8.00 (s, 1H), 7.96 (m, 2H), 7.88 (s, 1H), 7.70 (d, 1H), 7.50 (d, 1H), 7.47 (d, 1H), 7.37 (d, 1H), 7.21 (t, 1H), 6.63 (t, 1H), 6.49 (d, 1H), 4.57 (d, 2H), and 1.34 ppm (s, 9H); ES-MS m/z 483.5 [M+H]+, HPLC RT (min) 3.97.

### Example 82

### Ethyl 4-{[(2-{[(2,2-difluoro-1,3-benzodioxol-5-yl)amino]carbonyl}phenyl)amino]methyl}pyridine-2-carboxylate

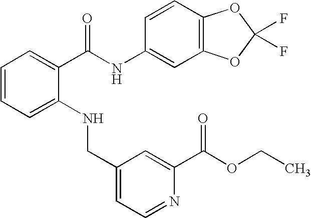

The procedures of Hadri, A. E.; Leclerc, G. J. Heterocyclic Chem. 1993, 30, 631-635 were used to prepare ethyl 4-(bromomethyl)pyridine-2-carboxylate. A solution of this material (450 mg, 1.84 mmol) and NaI (276 mg, 1.84 mmol) in DMF (5 mL) was stirred for 2 minutes and then a DMF (5 mL) solution of 2-amino-N-(2,2-difluoro-1,3-benzodioxol-5-yl)benzamide (414 mg, 1.42 mmol) was added quickly via syringe. The reaction was then heated with stirring under nitrogen at 90° C. for 16 hr. It was poured into water and extracted with EtOAc several times. The combined organic layer was dried (Na₂SO₄) and concentrated under reduced pressure to give ethyl 4-{[(2-{[(2,2-difluoro-1,3-benzodioxol-5-yl)amino]carbonyl}phenyl)amino]methyl}pyridine-2-carboxylate (200 mg, 31% yield): NMR (300 MHz, CD2Cl2) δ 8.60 (d, 1H), 8.36 (s, 1H), 8.15 (s, 1H), 8.05 (s, 1H), 7.74 (s, 1H), 7.55 (d, 1H), 7.48 (d, 1H), 7.26 (t, 1H), 7.18 (d, 1H), 7.06 (d, 1H), 6.64 (t, 1H), 6.48 (d, 1H), 4.57 (s, 2H), 4.40 (q, 2H), and 1.41 ppm (t, 3H); ES-MS m/z 456 [M+H]+, HPLC RT (min) 3.32.

### Example 83

### 4-{[(2-{[(2,2-Difluoro-1,3-benzodioxol-5-yl)amino]carbonyl}phenyl)amino]methyl}-N-[2-(methylsulfonyl)ethyl]pyridine-2-carboxamide

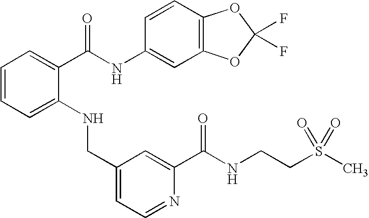

### Step 1: Preparation of 4-{[(2-{[(2,2-difluoro-1,3-benzodioxol-5-yl)amino]carbonyl}phenyl)amino]methyl}pyridine-2-carboxylic acid

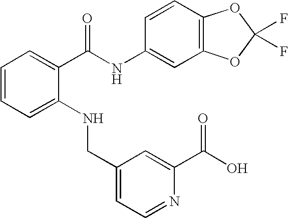

A solution of ethyl 4-{[(2-{[(2,2-difluoro-1,3-benzodioxol-5-yl)amino]carbonyl}phenyl)-amino]methyl}pyridine-2-carboxylate (455 mg, 1 mmol) in MeOH/H₂O/1N LiOH(aq) (7:2:1) (30 mL) was stirred 30 min at ambient temperature at which time LCMS analysis showed no starting material left. Hydrochloric acid (2N) was added dropwise until pH 7 was reached. The resultant solution was evaporated in vacuo to yield a white solid which was extracted with MeOH. The MeOH solution was evaporated in vacuo to give 4-{[(2-{[(2,2-difluoro-1,3-benzodioxol-5-yl)amino]carbonyl}phenyl)amino]methyl}pyridine-2-carboxylic acid. This material was used quickly in the next step.

### Step 2: Preparation of 4-{[(2-{[(2,2-difluoro-1,3-benzodioxol-5-yl)amino]carbonyl}phenyl)amino]methyl}-N-[2-(methylsulfonyl)ethyl]pyridine-2-carboxamide

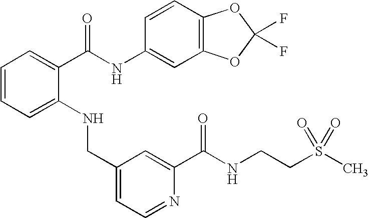

A solution of 4-{[(2-{[(2,2-difluoro-1,3-benzodioxol-5-yl)amino]carbonyl}phenyl)amino]methyl}pyridine-2-carboxylic acid (15 mg, 0.04 mmol), 2-(methylsulfonyl)-ethanamine (8.68 mg, 0.07 mmol) and PyBOP (36 mg, 0.07 mmol) in THF (5 mL) was stirred overnight. The resultant solution was diluted with water and extracted several times with dichloromethane. The combined organic layer was dried (Na₂SO₄) and concentrated under reduced pressure to give crude product which was purified by HPLC using the method of Example 77 to afford pure 4-{[(2-{[(2,2-difluoro-1,3-benzodioxol-5-yl)amino]carbonyl}phenyl)amino]methyl}-N-[2-(methylsulfonyl)ethyl]pyridine-2-carboxamide (8 mg, 43% yield): ¹H NMR (300 MHz, CD2Cl2) δ 8.58-8.62 (1H, m), 8.48 (1H, d), 8.09 (1H, s), 7.92 (1H, s), 7.63 (1H, s), 7.45-7.54 (2H, m), 7.21 (1H, t), 7.02-7.10 (2H, m), 6.68 (1H, t), 6.43 (1H, d), 4.57 (2H, s), 3.93 (2H, t), 3.30 (2H, t), 2.91 (3H, s); ES-MS m/z 533 [M+H]+, HPLC RT (min) 3.28.

### Example 84

### General Procedures for Preparation of N-(Substituted)-4-{[(2-{[(2,2-difluoro-1,3-benzodioxol-5-yl)amino]carbonyl}phenyl)amino]methyl}pyridine-2-carboxamides

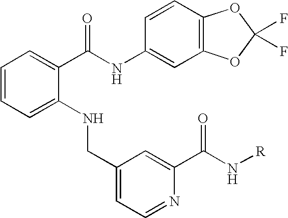

### Procedure 84-1:

Examples 89 through 91 in Table 2 were prepared by using the general procedure of Example 77 but substituting the appropriate amine of structure R—NH₂ for 1-(2-furyl)methanamine.

### Procedure 84-2:

Examples 92 through 101 in Table 2 were prepared by using the general procedure of Example 78 but substituting the appropriate amine of structure R—NH₂ for 1-(2,2-dimethyl-1,3-dioxolan-4-yl)methanamine.

### Procedure 84-3:

Examples 84 through 88 in Table 2 were prepared by using the general procedure of Example 83 but substituting the appropriate amine of structure R—NH₂ for 2-(methylsulfonyl)ethanamine.

### Procedure 84-4:

A stock solution of Methyl 4-{[(2-{[(2,2-difluoro-1,3-benzodioxol-5-yl)amino]carbonyl}phenyl)amino]methyl}pyridine-2-carboxylate in methanol (1.995 g, 4.51 mmol, in 90.2 mL MeOH, 0.05 M) was prepared. A portion (1960 μL, 0.098 mmol) of this stock solution was pipeted into an EPA vial containing a weighed amount of the amine of structure R—NH₂ (0.40 mmol). The reaction mixture was heated to 65° C. and shaken in a J-Kem block. The reaction mixtures were cooled, filtered, reformated into a 96-well MTP, and purified by Preparative LC/MS (Symmetry 5 um 30×75; ACN-Water with 0.1% TFA; 10% ACN to 90% ACN gradient). The fractions were evaporated in the Mega, reconsitituted into 1.5 mL DMSO, and like fractions were pooled using the Tecan. After drying in the speedvac, the vials were weighed and the products were characterized by LC/MS and NMR. The structures, names and LC/MS data of Examples 102 through 133 which were prepared by this method are shown in Table 2.

TABLE 2

| | | | | LCMS |
|---|---|---|---|---|
| Entry | | | LC-MS | RT |
| No. | Structure | Chemical Name | [M + H]+ | (min) |
| 84 | 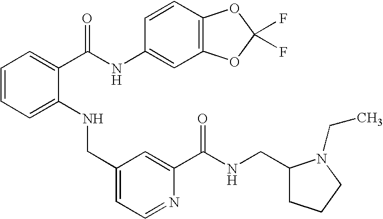 | 4-{[(2-{[(2,2-difluoro-1,3-benzodioxol-5-yl)amino]carbonyl}phenyl)amino]methyl}-N-[(1-ethylpyrrolidin-2-yl)methyl]pyridine-2-carboxamide; | 538 | 2.85 |
| 85 | 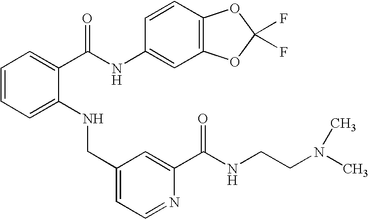 | 4-{[(2-{[(2,2-difluoro-1,3-benzodioxol-5-yl)amino]carbonyl}phenyl)amino]methyl}-N-[2-(dimethylamino)ethyl]pyridine-2-carboxamide; | 498 | 2.74 |
| 86 | 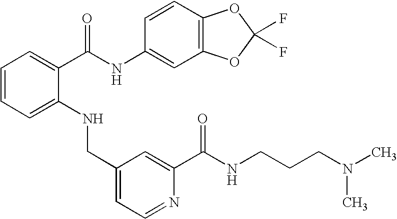 | 4-{[(2-{[(2,2-difluoro-1,3-benzodioxol-5-yl)amino]carbonyl}phenyl)amino]methyl}-N-[3-(dimethylamino)propyl]pyridine2-carboxamide; | 512 | 2.76 |
| 87 | 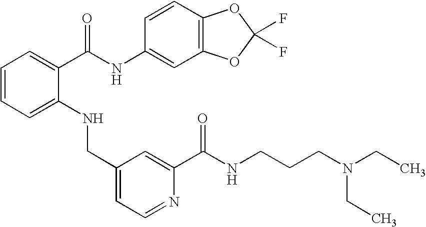 | N-[3-(diethylamino)propyl]-4-{[(2-{[(2,2-difluoro-1,3-benzodioxol-5-yl)amino]carbonyl}phenyl)amino]methyl}pyridine-2-carboxamide; | 540 | 2.83 |
| 88 | 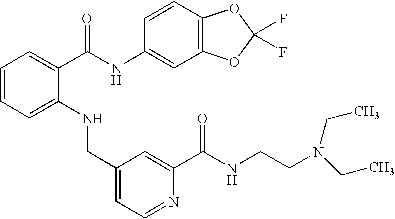 | N-[2-(diethylamino)ethyl]-4-{[(2{[(2,2-difluoro-1,3-benzodioxol-5-yl)amino]carbonyl}phenyl)amino]methyl}pyridine-2-carboxamide; | 526 | 2.83 |
| 89 | 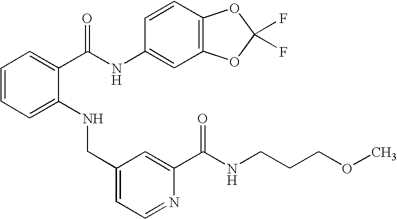 | 4-{[(2-{[(2,2-difluoro-1,3-benzodioxol-5-yl)amino]carbonyl}phenyl)amino]methyl}-N-(3-methoxypropyl)pyridine-2-carboxamide; | 499.2 | 3.8 |
| 90 | 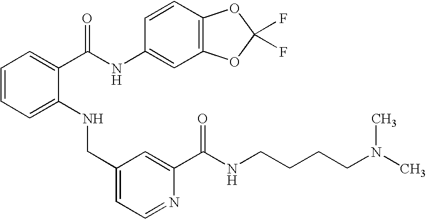 | 4-{[(2-{[(2,2-difluoro-1,3-benzodioxol-5-yl)amino]carbonyl}phenyl)amino]methyl}-N-[4-(dimethylamino)butyl]pyridine-2-carboxamide; | 526.3 | 2.73 |
| 91 | 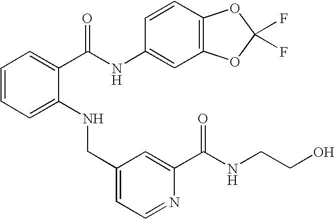 | 4-{[(2-{[(2,2-difluoro-1,3-benzodioxol-5-yl)amino]carbonyl}phenyl)amino]methyl}-N-(2-hydroxyethyl)pyridine-2-carboxamide | 271 | 3.09 |
| 92 | 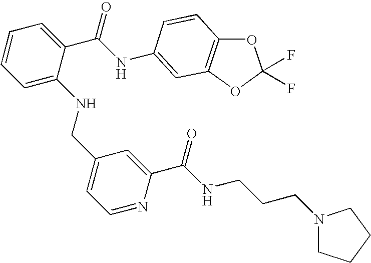 | 4-{[(2-{[(2,2-difluoro-1,3-benzodioxol-5-yl)amino]carbonyl}phenyl)amino]methyl}-N-(3-pyrrolidin-1-ylpropyl)pyridine-2-carboxamide; | 538.4 | 2.78 |
| 93 | 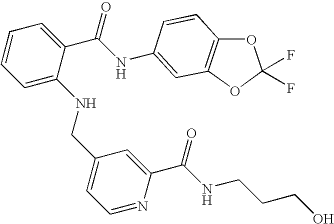 | 4-{[(2-{[(2,2-difluoro-1,3-benzodioxol-5-yl)amino]carbonyl}phenyl)amino]methyl}-N-(3-hydroxypropyl)pyridine-2-carboxamide; | 485.4 | 3.05 |
| 94 | 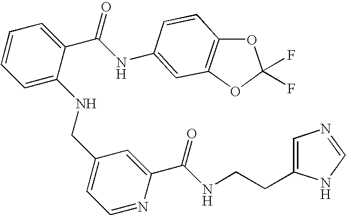 | 4-{[(2-{[(2,2-difluoro-1,3-benzodioxol-5-yl)amino]carbonyl}phenyl)amino]methyl}-N-[2-(1H-imidazol-5-yl)ethyl]pyridine-2-carboxamide; | 521.3 | 2.77 |
| 95 | 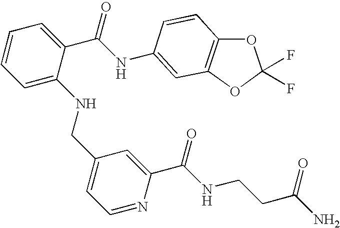 | N-(3-amino-3-oxopropyl)-4-{[(2{[(2,2-difluoro-1,3-benzodioxol-5-yl)amino]carbonyl}phenyl)amino]methyl}pyridine-2-carboxamide; | 498.2 | 3 |
| 96 | 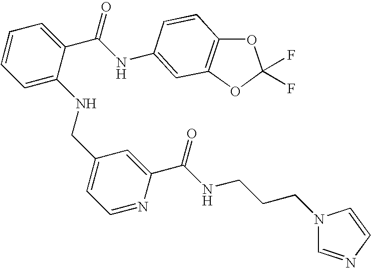 | 4-{[(2-{[(2,2-difluoro-1,3-benzodioxol-5-yl)amino]carbonyl}phenyl)amino]methyl}-N-[3-(1H-imidazol-1-yl)propyl]pyridine-2-carboxamide; | 535.3 | 2.77 |
| 97 | 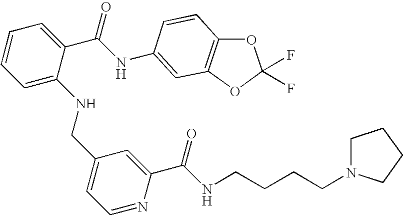 | 4-{[(2-{[(2,2-difluoro-1,3-benzodioxol-5-yl)amino]carbonyl}phenyl)amino]methyl}-N-(4-pyrrolidin-1-ylbutyl)pyridine-2-carboxamide; | 552.5 | 2.8 |
| 98 | 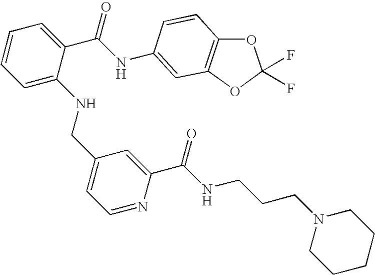 | 4-{[(2-{[(2,2-difluoro-1,3-benzodioxol-5-yl)amino]carbonyl}phenyl)amino]methyl}-N-(3-piperidin-1-ylpropyl)pyridine-2-carboxamide; | 552.4 | 2.8 |
| 99 | 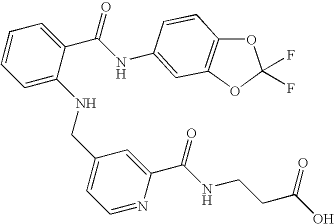 | N-[(4-{[(2-{[(2,2-difluoro-1,3-benzodioxol-5-yl)amino]carbonyl}phenyl)amino]methyl}pyridin-2-yl)carbonyl]beta-alanine; | 499.2 | 3.04 |
| 100 | 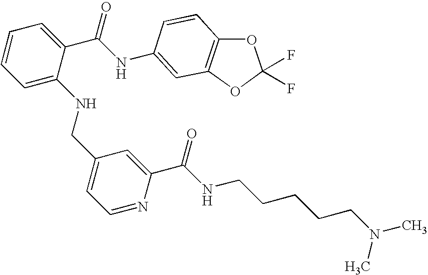 | 4-{[(2-{[(2,2-difluoro-1,3-benzodioxol-5-yl)amino]carbonyl}phenyl)amino]methyl)-N-[5-(dimethylamino)pentyl]pyridine2-carboxamide; | 540.5 | 2.79 |
| 101 | 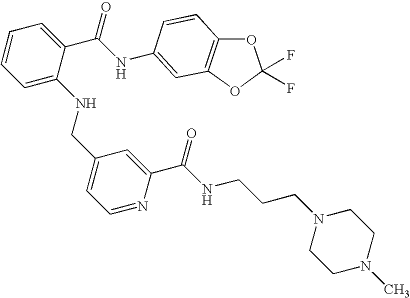 | 4-{[(2-{[(2,2-difluoro-1,3-benzodioxol-5-yl)amino]carbonyl}phenyl)amino]methyl}-N-[3-(4-methylpiperazin-1-yl)propyl]pyridine-2-carboxamide; | 567.4 | 2.56 |
| 102 | 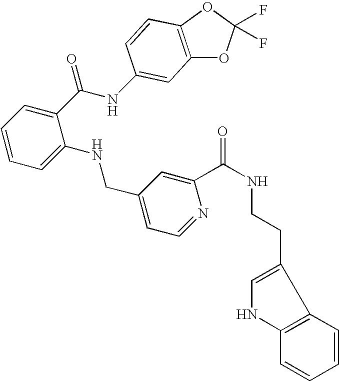 | 4-{[(2-{[(2,2-difluoro-1,3-benzodioxol-5-yl)amino]carbonyl}phenyl)amino]methyl}-N-[2-(1H-indol-3-yl)ethyl]pyridine-2-carboxamide; | 569.19 | 4.2 |
| 103 | 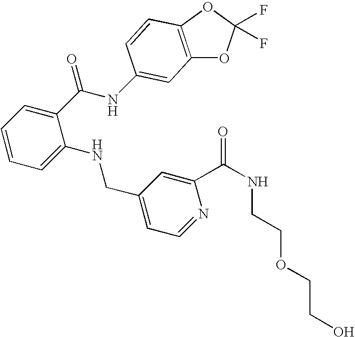 | 4-{[(2-{[(2,2-difluoro-1,3-benzodioxol-5-yl)amino]carbonyl}phenyl)amino]methyl}-N-[2-(2-hydroxyethoxy)ethyl]pyridine-2carboxamide; | 514.17 | 3.57 |
| 104 | 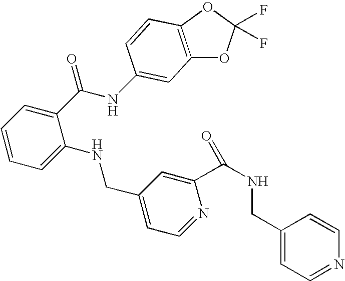 | 4-{[(2-{[(2,2-difluoro-1,3-benzodioxol-5-yl)amino]carbonyl}phenyl)amino]methyl}-N-(pyridin-4-ylmethyl)pyridine-2-carboxamide; | 517.16 | 3.1 |
| 105 | 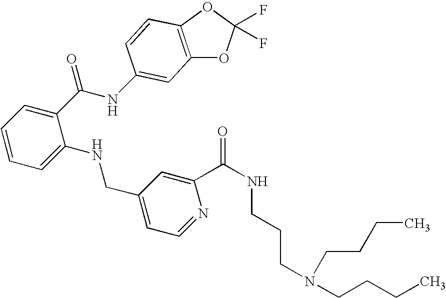 | N-[3-(dibutylamino)propyl]-4-{[(2-{[(2,2-difluoro-1,3-benzodioxol-5-yl)amino]carbonyl}phenyl)amino]methyl}pyridine-2-carboxamide; | 595.3 | 3.36 |
| 106 | 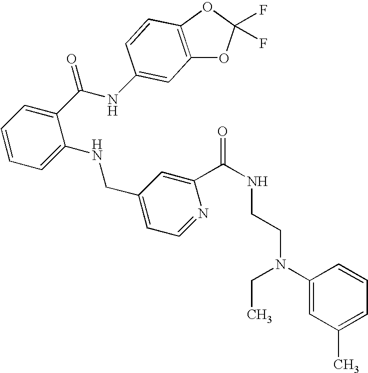 | 4-{[(2-{[(2,2-difluoro-1,3-benzodioxol-5-yl)amino]carbonyl}phenyl)amino]methyl}-N-{2-[ethyl(3-methylphenyl)amino]ethyl}pyridine-2-carboxamide; | 587.23 | 3.64 |
| 107 | 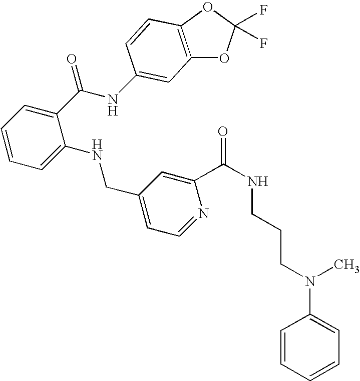 | 4-{[(2-{[(2,2-difluoro-1,3-benzodioxol-5-yl)amino]carbonyl}phenyl)amino]methyl}-N-{3-[methyl(phenyl)amino]propyl}pyridine-2-carboxamide; | 573.22 | 3.52 |
| 108 | 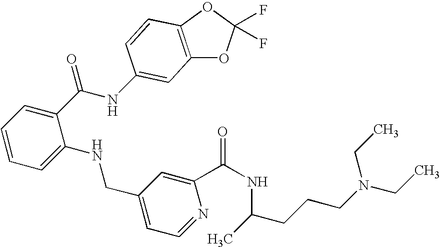 | N-[4-(diethylamino)-1-methylbutyl]-4-{[(2-{[(2,2-difluoro-1,3-benzodioxol-5-yl)amino]carbonyl}phenyl)amino]methyl}pyridine-2-carboxamide; | 567.27 | 3.19 |
| 109 | 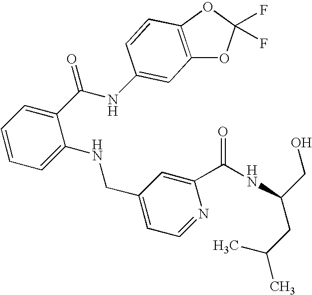 | 4-{[(2-{[(2,2-difluoro-1,3-benzodioxol-5-yl)amino]carbonyl}phenyl)amino]methyl}-N-[1-(hydroxymethyl)-3-methylbutyl]pyridine-2-carboxamide; | 526.2 | 4.02 |
| 110 | 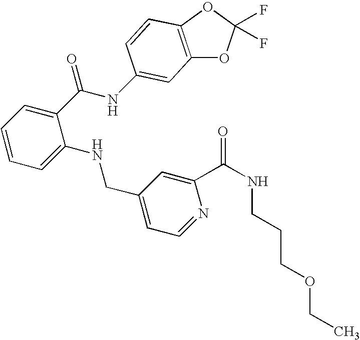 | 4-{[(2-{[(2,2-difluoro-1,3-benzodioxol-5-yl)amino]carbonyl}phenyl)amino]methyl}-N-(3-ethoxypropyl)pyridine-2-carboxamide | 512.19 | 4.03 |
| 111 | 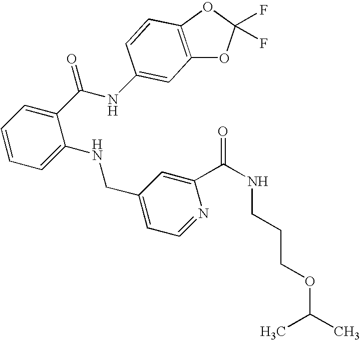 | 4-{[(2-{[(2,2-difluoro-1,3-benzodioxol-5-yl)amino]carbonyl}phenyl)amino]methyl}-N-(3-isopropoxypropyl)pyridine-2-carboxamide; | 526.2 | 4.19 |
| 112 | 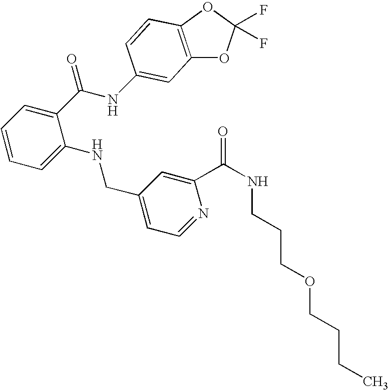 | N-(3-butoxypropyl)-4-{[(2-{[(2,2difluoro-1,3-benzodioxol-5-yl)amino]carbonyl}phenyl)amino]methyl}pyridine-2-carboxamide; | 540.22 | 4.35 |
| 113 | 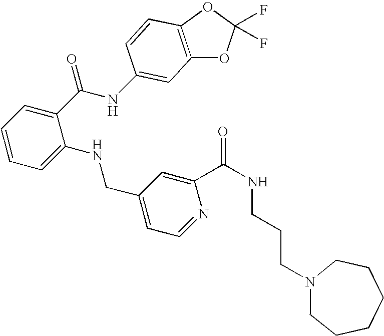 | N-(3-azepan-1-ylpropyl)-4-{[(2-{[(2,2-difluoro-1,3-benzodioxol-5-yl)amino]carbonyl}phenyl)amino]methyl}pyridine-2-carboxamide; | 565.25 | 3.18 |
| 114 | 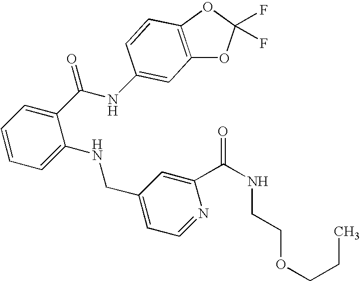 | 4-{[(2-{[(2,2-difluoro-1,3-benzodioxol-5-yl)amino]carbonyl}phenyl)amino]methyl}-N-(2-propoxyethyl)pyridine-2-carboxamide | 512.19 | 4.16 |
| 115 | 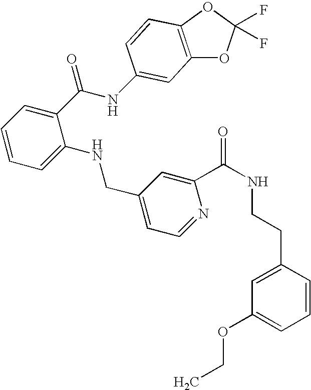 | 4-{[(2-{[(2,2-difluoro-1,3-benzodioxol-5-yl)amino]carbonyl}phenyl)amino]methyl}-N-[2-(3-ethoxyphenyl)ethyl]pyridine-2-carboxamide; | 574.2 | 4.38 |
| 116 | 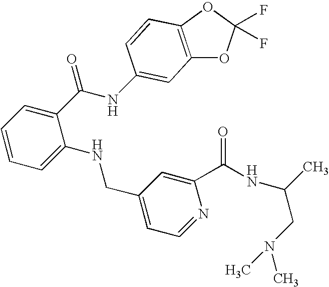 | 4-{[(2-{[(2,2-difluoro-1,3-benzodioxol-5-yl)amino]carbonyl}phenyl)amino]methyl}-N-[2-(dimethylamino)-1-methylethyl]pyridine-2-carboxamide | 511.2 | 3.08 |
| 117 | 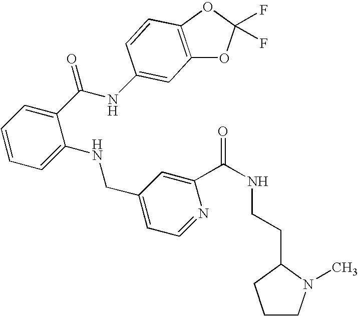 | 4-{[(2-{[(2,2-difluoro-1,3-benzodioxol-5-yl)amino]carbonyl}phenyl)amino]methyl}-N-[2-(1-methylpyrrolidin-2-yl)ethyl]pyridine-2-carboxamide; | 537.22 | 3.09 |
| 118 | 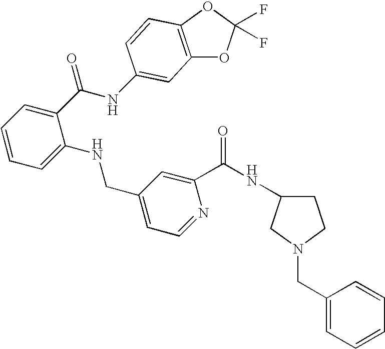 | N-(1-benzylpyrrolidin-3-yl)-4-{[(2-{[(2,2-difluoro-1,3-benzodioxol-5-yl)amino]carbonyl}phenyl)amino]methyl}pyridine-2-carboxamide | 585.22 | 3.25 |
| 119 | 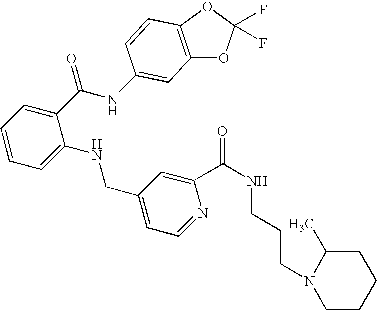 | 4-{[(2-{[(2,2-difluoro-1,3-benzodioxol-5-yl)amino]carbonyl}phenyl)amino]methyl}-N-[3-(2-methylpiperidin-1-yl)propyl]pyridine-2-carboxamide; | 565.25 | 3.16 |
| 120 | 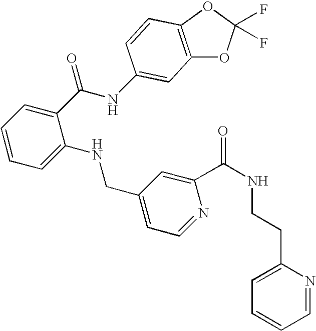 | 4-{[(2-{[(2,2-difluoro-1,3-benzodioxol-5-yl)amino]carbonyl}phenyl)amino]methyl}-N-(2-pyridin-2-ylethyl)pyridine-2-carboxamide; | 531.17 | 3.12 |
| 121 | 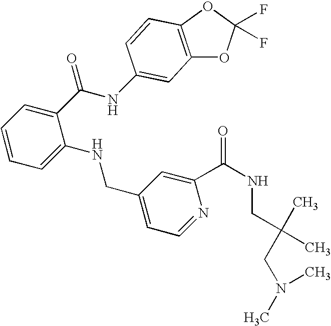 | 4-{[(2-{[(2,2-difluoro-1,3-benzodioxol-5-yl)amino]carbonyl}phenyl)amino]methyl}-N-[3-(dimethylamino)-2,2-dimethylpropyl]pyridine-2-carboxamide; | 539.23 | 3.13 |
| 122 | 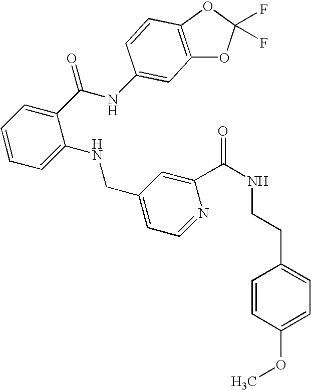 | 4-{[(2-{[(2,2-difluoro-1,3-benzodioxol-5-yl)amino]carbonyl}phenyl)amino]methyl}-N-[2-(4-methoxyphenyl)ethyl]pyridine-2-carboxamide; | 560.19 | 4.25 |
| 123 | 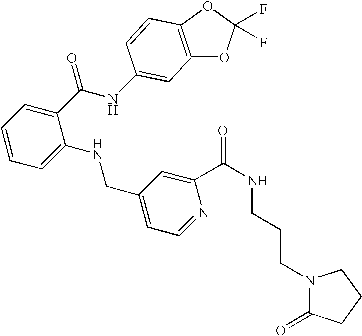 | 4-{[(2-{[(2,2-difluoro-1,3-benzodioxol-5-yl)amino]carbonyl}phenyl)amino]methyl}-N-[3-(2-oxopyrrolodin-1-yl)propyl]pyridine-2-carboxamide; | 551.2 | 3.65 |
| 124 | 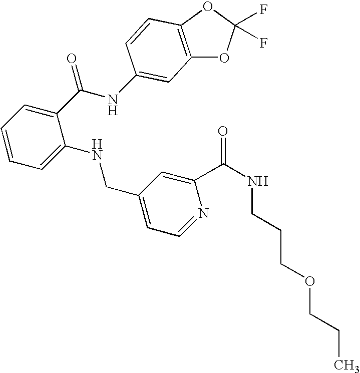 | 4-{[(2-{[(2,2-difluoro-1,3-benzodioxol-5-yl)amino]carbonyl}phenyl)amino]methyl}-N-(3-propoxypropyl)pyridine-2-carboxamide; | 526.2 | 4.23 |
| 125 | 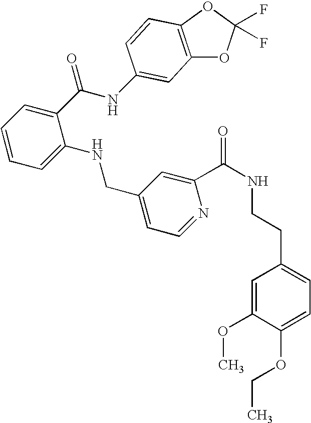 | 4-{[(2-{[(2,2-difluoro-1,3-benzodioxol-5-yl)amino]carbonyl}phenyl)amino]methyl}-N-[2-(4-ethoxy-3-methoxyphenyl)ethyl]pyridine-2-carboxamide; | 604.21 | 4.22 |
| 126 | 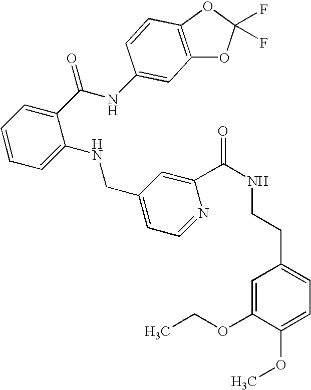 | 4-{[(2-{[(2,2-difluoro-1,3-benzodioxol-5-yl)amino]carbonyl}phenyl)amino]methyl}-N-[2-(3-ethoxy-4-methoxyphenyl)ethyl]pyridine-2-carboxamide; | 604.21 | 4.2 |
| 127 | 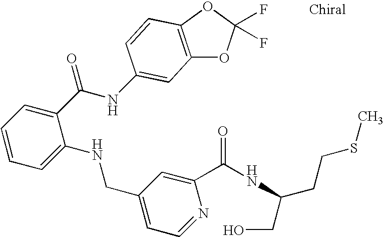 | 4-{[(2-{[(2,2-difluoro-1,3-benzodioxol-5-yl)amino]carbonyl}phenyl)amino]methyl}-N-[(1S)-1-(hydroxymethyl)-3-(methylthio)propyl]pyridine-2-carboxamide; | 544.16 | 3.83 |
| 128 | 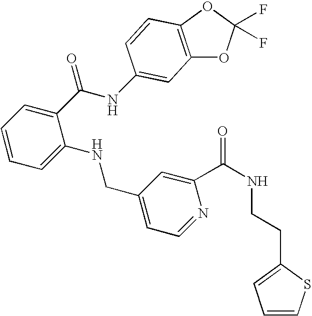 | 4-{[(2-{[(2,2-difluoro-1,3-benzodioxol-5-yl)amino]carbonyl}phenyl)amino]methyl}-N-[2-(2-thienyl)ethyl]pyridine-2-carboxamide; | 536.13 | 4.22 |
| 129 | 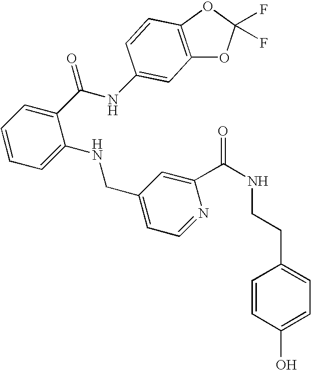 | 4-{[(2-{[(2,2-difluoro-1,3-benzodioxol-5-yl)amino]carbonyl}phenyl)amino]methyl}-N-[2-(4-hydroxyphenyl)ethyl]pyridine-2-carboxamide; | 546.17 | 3.92 |
| 130 | 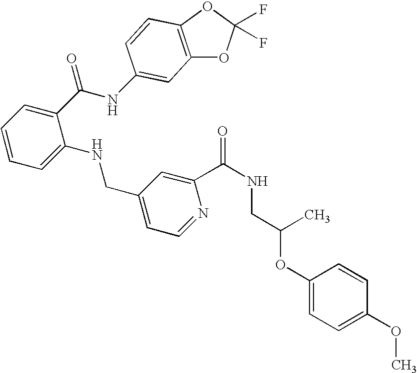 | 4-{[(2-{[(2,2-difluoro-1,3-benzodioxol-5-yl)amino]carbonyl)phenyl)amino]methyl}-N-[2-(4-methoxyphenoxy)propyl]pyridine-2-carboxamide; | 590.2 | 4.29 |
| 131 | 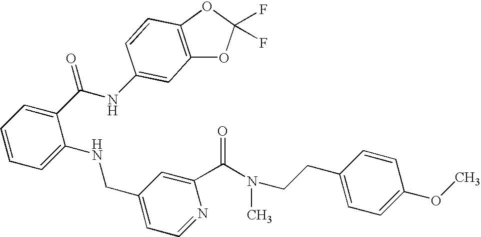 | 4-{[(2-{[(2,2-difluoro-1,3-benzodioxol-5-yl)amino]carbonyl}phenyl)amino]methyl}-N-[2-(4-methoxyphenyl)ethyl]-N-methylpyridine-2-carboxamide | 588.22 | 4.34 |
| 132 | 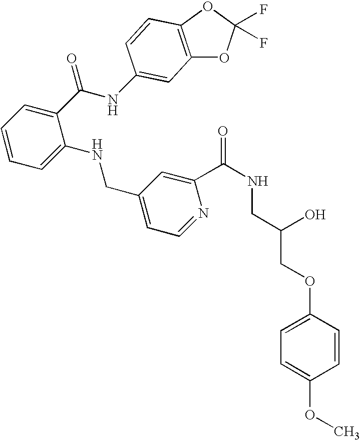 | 4-{[(2-{[(2,2-difluoro-1,3-benzodioxol-5-yl)amino]carbonyl}phenyl)amino]methyl}-N-[2-hydroxy-3-(4-methoxyphenoxy)propyl]pyridine-2-carboxamide; | 606.19 | 3.91 |
| 133 | 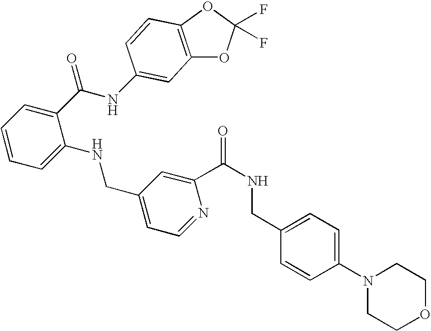 | 4-{[(2-{[(2,2-difluoro-1,3-benzodioxol-5-yl)amino]carbonyl}phenyl)amino]methyl}-N-(4-morpholin-4-ylbenzyl)pyridine-2-carboxamide; | 601.21 | 3.89 |

### Example 134

### Preparation of N-(2,2-difluoro-1,3-benzodioxol-5-yl)-2-[({2-[(2-hydroxyethyl)amino]pyridin-4-yl}methyl)amino]benzamide trifluoroacetate (salt)

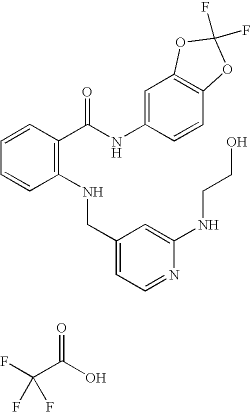

### Step 1: Preparation of 2-{[(2-chloropyridin-4-yl)methyl]amino}-N-(2,2-difluoro-1,3-benzodioxol-5-yl)benzamide

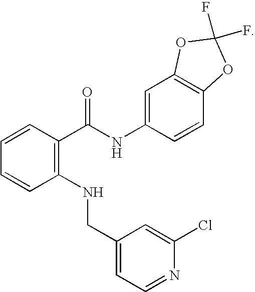

A solution of 2-amino-N-(2,2-difluoro-1,3-benzodioxol-5-yl)benzamide intermediate W (22.85 g, 9.77 mmol) and 2-chloro-4-(chloromethyl)pyridine intermediate T (3.25 g, 14.66 mmol) in DMF (10 mL) was treated with N,N-diisopropylethylamine (2.04 ml, 11.73 mmol) and sodium iodide (1.47 g, 9.77 mmol), and the reaction mixture was heated to 60° C. for 48 h. The reaction mixture was diluted with ethyl acetate and washed with saturated NaHCO₃, followed by water, then brine. The organic layer was dried over Na₂SO₄ and concentrated in vacuo. The crude residue was crystallized from EtOH/water to yield 3.64 g (89.1%) of product as a light brown powder.

¹H NMR (300 MHz, CD₃CN) δ 8.80 (bs, 1H), 8.29 (d, 1H), 8.03 (bs, 1H), 7.77 (s, 1H), 7.69 (d, 1H), 7.23-7.40 (m, 4H), 7.20 (d, 1H), 6.73 (t, 1H), 6.58 (d, 1H), 4.52 (s, 2H); ES-MS m/z 417.9 [M+H]⁺, HPLC RT (min) 3.77.

### Step 2: Preparation of N-(2,2-difluoro-1,3-benzodioxol-5-yl)-2-[({2-[(2-hydroxyethyl)amino]pyridin-4-yl}methyl)amino]benzamide trifluoroacetate (salt)

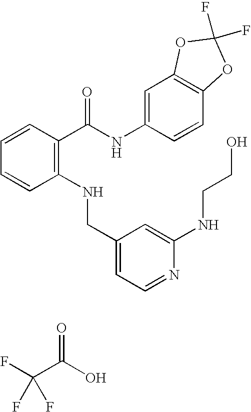

A solution of 2-{[(2-chloropyridin-4-yl)methyl]amino}-N-(2,2-difluoro-1,3-benzodioxol-5-yl)benzamide (step 1) (200.0 mg, 0.48 mmol) in pyridine (3 mL) was treated with ethanolamine (11.0 mL, 16.56 mmol) and heated to 200° C. in a sealed tube for 12 hrs. The reaction mixture was then allowed to cool to room temperature. It was then diluted with water and extracted with EtOAc. The organic extracts were washed with water, dried over Na₂SO₄, and concentrated in vacuo. Purification of the crude residue by HPLC (10-90% MeCN in water containing 0.1% TFA gradient) gave 68.0 mg (25.5%) of the title compound as the TFA salt.

¹H NMR (300 MHz, CD₃CN) δ 8.85 (bs, 1H), 7.68-7.80 (m, 3H), 7.28-7.38 (m, 2H), 7.20 (d, 1H), 6.97 (s, 1H), 6.72-6.82 (m, 2H), 6.57 (d, 1H), 4.51 (s, 2H), 3.69 (t, 2H), 3.40 (m, 2H); ES-MS m/z 443.2 [M+H]⁺, HPLC RT (min) 2.83.

Examples 135-150 were prepared using the same method as Example 134 using the appropriate commercially available amine starting material instead of ethanolamine.

| | | | | LCMS |
|---|---|---|---|---|
| Entry | | | LC-MS | RT |
| No. | Structure | Chemical Name | [M + H]+ | (min) |
| 135 | 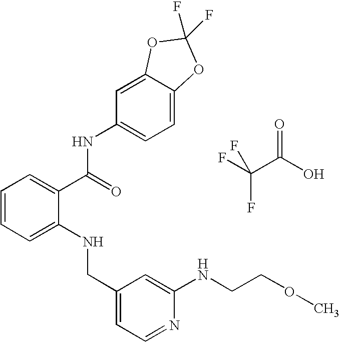 | N-(2,2-difluoro-1,3-benzodioxol-5-yl)-2-[({2-[(2-methoxyethyl)amino]pyridin-4-yl}methyl)amino]benzamide trifluoroacetate; | 457.2 | 2.67 |
| 136 | 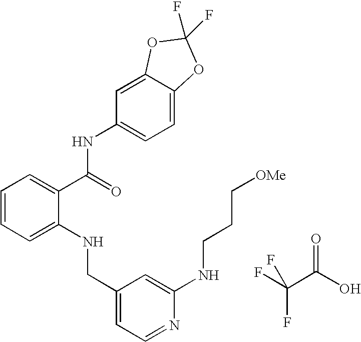 | N-(2,2-difluoro-1,3-benzodioxol-5-yl)-2-[({2-[(3-methoxypropyl)amino]pyridin-4-yl}methyl)amino]benzamide trifluoroacetate; | 471.3 | 2.98 |
| 137 | 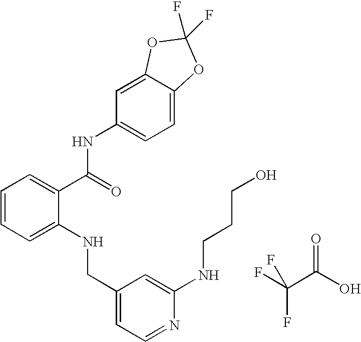 | N-(2,2-difluoro-1,3-benzodioxol-5-yl)-2-[({2-[(3-hydroxypropyl)amino]pyridin-4-yl}methyl)amino]benzamide trifluoroacetate(salt); | 457.1 | 2.52 |
| 138 | 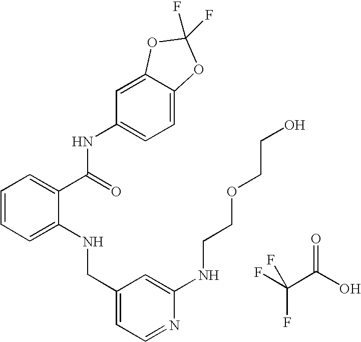 | N-(2,2-difluoro-1,3-benzodioxol-5-yl)-2-{[(2-{[2-(2-hydroxyethoxy)ethyl]amino}pyridin-4-yl)methyl]amino}benzamide trifluoroacetate(salt); | 487.3 | 2.81 |
| 139 | 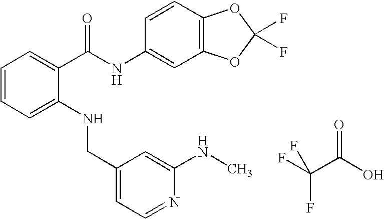 | N-(2,2-difluoro-1,3-benzodioxol-5-yl)-2-({[2-(methylamino)pyridin-4-yl]methyl}amino)benzamide trifluoroacetate; | 413.2 | 2.67 |
| 140 | 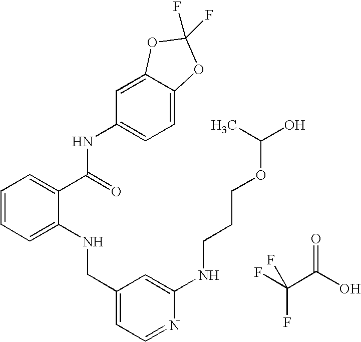 | N-(2,2-difluoro-1,3-benzodioxol-5-yl)-2-[({2-[(3-isopropoxypropyl)amino]pyridin-4-yl}methyl)amino]benzamide trifluoroacetate; | 499.2 | 2.78 |
| 141 | 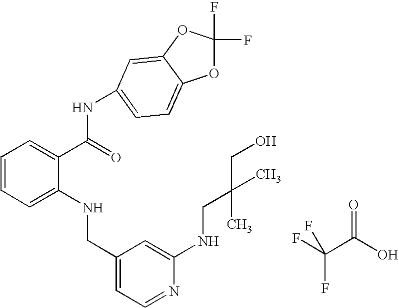 | N-(2,2-difluoro-1,3-benzodioxol-5-yl)-2-[({2-[(3-hydroxy-2,2-dimethylpropyl)amino]pyridin-4-yl}methyl)amino]benzamide trifluoroacetate(salt); | 485.2 | 2.65 |
| 142 | 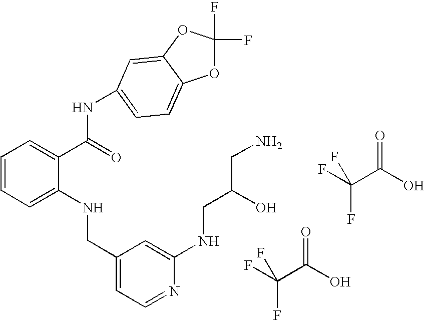 | 2-[({2-[(3-amino-2-hydroxypropyl)amino]pyridin-4-yl}methyl)amino]-N-(2,2-difluoro-1,3-benzodioxol-5-yl)benzamidebis(trifluoroacetate)(salt); | 472.2 | 2.15 |
| 143 | 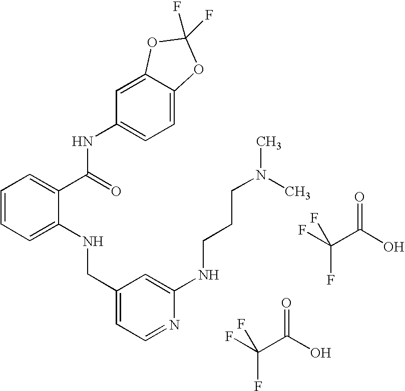 | N-(2,2-difluoro-1,3-benzodioxol-5-yl)-2-{[(2-{[3-(dimethylamino)propyl]amino}pyridin-4-yl)methyl]amino}benzamidebis(trifluoroacetate); | 484.1 | 2.19 |
| 144 | 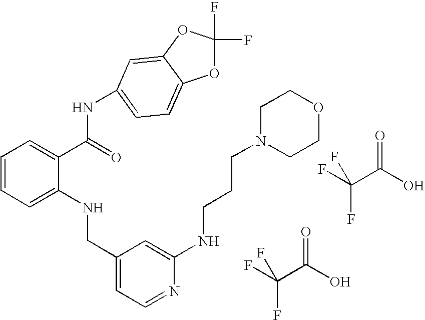 | N-(2,2-difluoro-1,3-benzodioxol-5-yl)-2-[({2-[(3-morpholin-4-ylpropyl)amino]pyridin-4-yl}methyl)amino]benzamidebis(trifluoroacetate); | 526.1 | 2.18 |
| 145 | 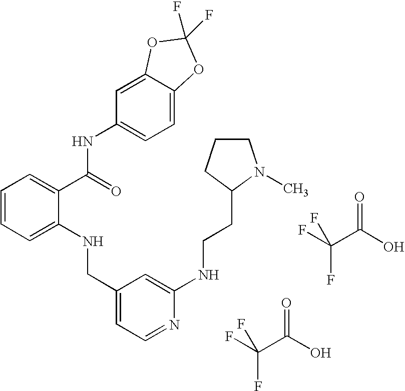 | N-(2,2-difluoro-1,3-benzodioxol-5-yl)-2-{[(2-{[2-(1-methylpyrrolidin-2-yl)ethyl]amino}pyridin-4-yl)methyl]amino}benzamidebis(trifluoroacetate); | 510.2 | 2.22 |
| 146 | 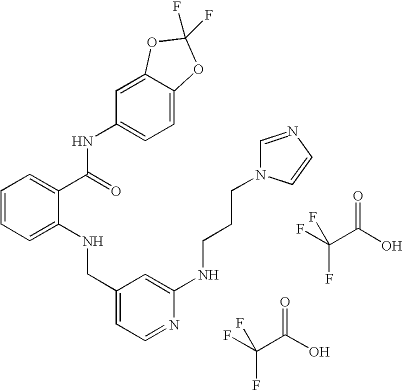 | N-(2,2-difluoro-1,3-benzodioxol-5-yl)-2-{[(2-{[3-(1H-imidazol-1-yl)propyl]amino}pyridin-4-yl)methyl]amino}benzamidebis(trifluoroacetate); | 507.1 | 2.2 |
| 147 | 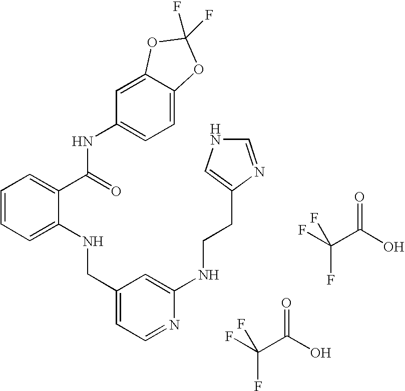 | N-(2,2-difluoro-1,3-benzodioxol-5-yl)-2-{[(2-{[2-(1H-imidazol-4-yl)ethyl]amino}pyridin-4-yl)methyl]amino}benzamidebis(trifluoroacetate); | 493.2 | 2.22 |
| 148 | 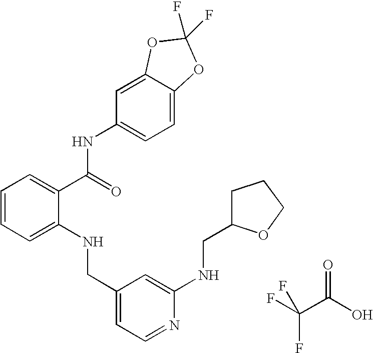 | N-(2,2-difluoro-1,3-benzodioxol-5-yl)-2-[({2-[(tetrahydrofuran-2-ylmethyl)amino]pyridin-4-yl}methyl)amino]benzamide trifluoroacetate; | 483.2 | 2.65 |
| 149 | 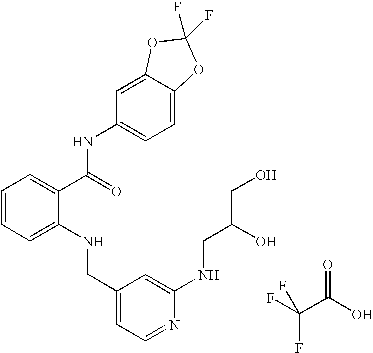 | N-(2,2-difluoro-1,3-benzodioxol-5-yl)-2-[({2-[(2,3-dihydroxypropyl)amino]pyridin-4-yl}methyl)amino]benzamidetrifluoroacetate (salt); | 473.3 | 2.48 |
| 150 | 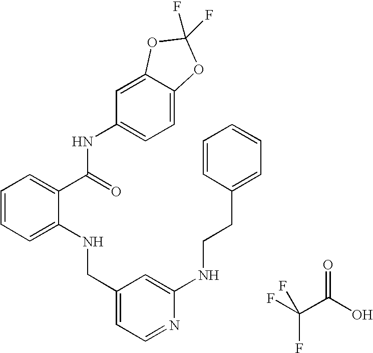 | N-(2,2-difluoro-1,3-benzodioxol-5-yl)-2-[({2-[(2-phenylethyl)amino]pyridin-4-yl}methyl)amino]benzamidetrifluoroacetate; | 503.2 | 3.22 |

### Example 151

### Preparation of N-(2,2-difluoro-1,3-benzodioxol-5-yl)-2-[({2-[(2-methoxypropanoyl)amino]pyridin-4yl}methyl)amino]benzamide

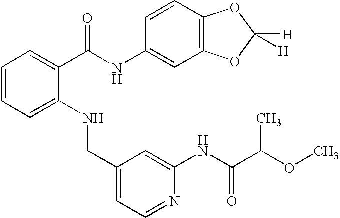

To a solution of 2-amino-N-(2,2-difluoro-1,3-benzodioxol-5-yl)benzamide (180 mg, 0.618 mmol) (intermediate W) in DMF (1 mL) was added N-[4-(chloromethyl)pyridin-2-yl]-2-methoxypropanamide (1.55 mg, 0.68 mmol) (intermediate M) followed by triethylamine (125 mg, 124 mmol). The reaction was degassed under high vacuum. The flask was then wrapped in foil to minimize the amount of light entering the reaction, then placed under a nitrogen atmosphere. Di(tert)butyl-4-methyphenol (BHT) (6.79 mg, 0.031 mmol) was added followed by sodium iodide (111 mg, 0.742 mmol). The reaction was again degassed under high vacuum then blanketed with nitrogen. The reaction was heated at 60° C. for 2 hours, and then cooled to room temperature. The reaction mixture was partitioned between EtOAc and saturated aqueous sodium bicarbonate. The aqueous layer was extracted with EtOAc two times. The combined organics were washed with saturated aqueous sodium bicarbonate 5 times to remove DMF. The organic layer is dried with sodium sulfate the concentrated under reduced pressure. The residue was chromatographed with 30% EtOAc/hexanes to yield pure N-(2,2-difluoro-1,3-benzodioxol-5-yl)-2-[({2-[(2-methoxypropanoyl)amino]-pyridin-4yl}methyl)amino]benzamide as a solid. (127 mg, 42%) ¹H NMR (DMSO-δ₆) δ 8.22 (d, 1H), 8.11 (s, 1H), 7.92 (t, 1H), 7.81 (s, 1H), 7.68 (d, 1H), 7.47 (m, 1H), 7.44 (m, 1H), 7.38 (s, 1H), 7.36 (s, 1H), 7.23 (t, 1H), 7.07 (d, 1H), 6.65 (t, 1H), 6.57 (d, 1H), 4.47 (d, 2H), 3.96 (q, 1H), 3.26 (s, 3H), 1.25 (d, 3H). LCMS: 485.2 [M+H]⁺ RT 3.24 min.

Examples 152-160 were prepared using the procedure for example 151 and using intermediate W as one of the starting materials and the corresponding intermediate selected from intermediates I-N as the other starting material.

| | | | | LCMS |
|---|---|---|---|---|
| Entry | | | LC-MS | RT |
| No. | Structure | Chemical Name | [M + H]+ | (min) |
| 152 | 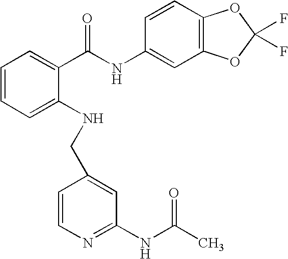 | 2-({[2-(acetylamino)pyridin-4-yl]methyl}amino)-N-(2,2-difluoro-1,3-benzodioxol-5-yl)benzamide; | 441.2 | 2.79 |
| 153 | 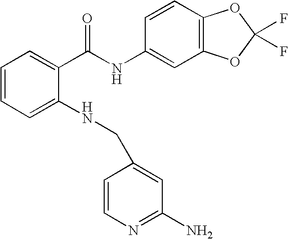 | 2-{[(2-aminopyridin-4-yl)methyl]amino}-N-(2,2-difluoro-1,3-benzodioxol-5-yl)benzamide; | 399.2 | 2.63 |
| 154 | 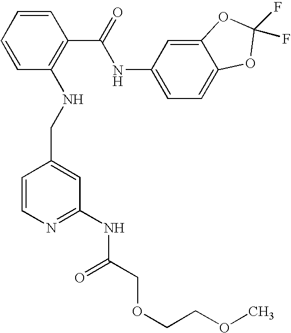 | N-(2,2-difluoro-1,3-benzodioxol-5-yl)-2-{[(2{[(2-methoxyethoxy)acetyl]amino}pyridin-4-yl)methyl]amino}benzamide; | 515.2 | 3.35 |
| 155 | 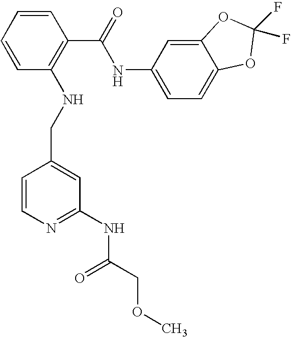 | N-(2,2-difluoro-1,3-benzodioxol-5-yl)-2-[({2-[(methoxyacetyl)amino]pyridin-4-yl}methyl)amino]benzamide; | 471.5 | 3.04 |
| 156 | 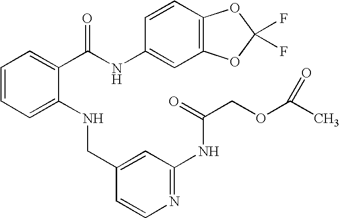 | 2-[(4-{[(2-{[(2,2-difluoro-1,3-benzodioxol-5-yl)amino]carbonyl}phenyl)amino]methyl}pyridin-2-yl)aminol-2-oxoethyl acetate; | 499.1 | 3.06 |
| 157 | 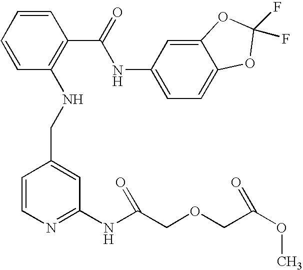 | methyl {2-[(4-{[(2-{[(2,2-difluoro-1,3-benzodioxol-5-yl)amino]carbonyl}phenyl)amino]methyl}pyridin-2-yl)amino[-2-oxoethoxy}acetate; | 529.2 | 3.07 |
| 158 | 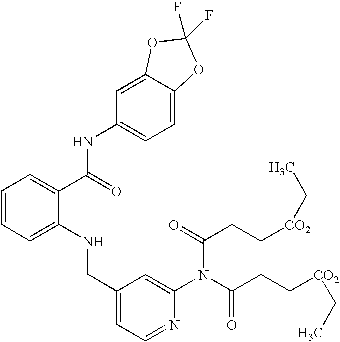 | diethyl 4,4′-[(4-{[(2-{[(2,2-difluoro-1,3-benzodioxol-5-yl)amino]carbonyl}phenyl)amino]methyl}pyridin-2-yl)imino]bis(4-oxobutanoate); | 655 | 3.76 |
| 159 | 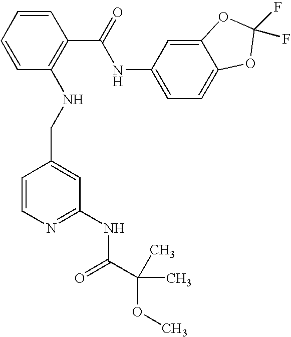 | N-(2,2-difluoro-1,3-benzodioxol-5-yl)-2-[({2-[(2-methoxy-2-methylpropanoyl)amino]pyridin-4-yl}methyl)amino]benzamide; | 499.1 | 3.55 |
| 160 | 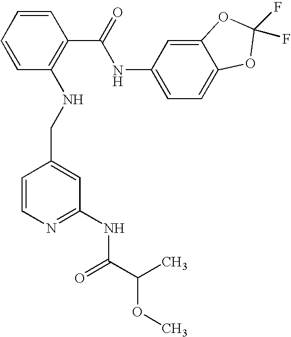 | N-(2,2-difluoro-1,3-benzodioxol-5-yl)-2-[({2-[(2-methoxypropanoyl)amino]pyridin-4-yl}methyl)amino]benzamide; | 485.2 | 3.24 |

### Example 161

### Preparation of 2-{[(2-aminopyridin-4-yl)methyl]amino}-N-(2,2-difluoro-1,3-benzodioxol-5-yl)benzamide

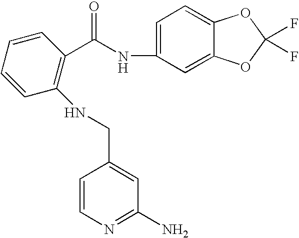

To a solution of 2-amino-N-(2,2-difluoro-1,3-benzodioxol-5-yl)benzamide (2.5 g, 8.56 mmol) (Intermediate W) and 4-(chloromethyl)pyridin-2-amine (1.70 g, 10.26 mmol) (Intermediate I step 1) in anhydrous DMF was added 2,6-di-tert-butyl-4-methylphenol (0.09 g, 0.42 mmol). The reaction mixture was degassed to remove oxygen and NaI (1.67 g, 11.12 mmol) was added. The reaction mixture was covered with aluminum foil and stirred at 60° C. for 18 h and cooled to room temperature. The reaction mixture was diluted with ethyl acetate (120 ml) and was washed with H₂O two times. The aqueous phase was back extracted with EtOAc. The combined organic layer was dried over Na₂SO₄ and concentrated to give a yellow crude oil. The crude was dissolved in CH₂Cl₂ (10 ml) and the product crashed out as a yellow solid. The solid product was collected by filtration and washed with minimal CH₂Cl₂. The filtrate was purified by column chromatography with MeOH in CH₂Cl₂ using a gradient from 0 to 12%. A total of 1.74 g (51%) of the title compound was obtained.

¹H NMR (DMSO-d₆) δ 10.31 (s, 1H), 7.81-7.93 (m, 4H), 7.68-7.70 (m, 1H), 7.39-7.47 (m, 2H), 7.23-7.29 (m, 1H), 6.49-6.68 (m, 5H) and 4.36 ppm (d, 2H); LC-MS 399.1[M+H]⁺, RT 2.61 min.

### Example 162

### Preparation of N-(2,2-difluoro-1,3-benzodioxol-5-yl)-2-{[(2-{[(ethylamino)carbonyl]amino}pyridin-4-yl)methyl]amino}benzamide

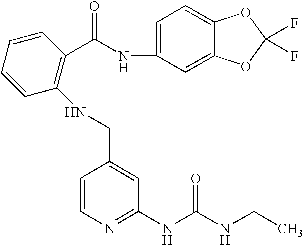

To a solution of 2-amino-N-(2,2-difluoro-1,3-benzodioxol-5-yl)benzamide (0.11 g, 0.36 mmol) (intermediate W) in dry DMF was added N-[4-(chloromethyl)pyridin-2-yl]-N′-ethylurea (0.10 g, 0.48 mmol) (intermediate P) and 2,6-Di-tert-butyl-4-methylphenol (0.004 g, 0.019 mmol). The reaction mixture was degassed to remove oxygen. Then NaI (0.073 g, 0.49 mmol) was added under N₂ and the flask was covered with aluminum foil and stirred for 5 min, The mixture was stirred at 60° C. for 18 h. The reaction was cooled to rt, diluted with EtOAc, washed with H₂O and brine and then dried over Na₂SO₄. The solvent was evaporated to give a yellow solid. The product that crushed out of CH₂Cl₂ was filtered and washed with CH₂Cl₂ three times to give N-(2,2-difluoro-1,3-benzodioxol-5-yl)-2-{[(2{[(ethylamino)carbonyl]amino}pyridin-4-yl)methyl]amino}benzamide as a solid (0.042 g, 23%).

¹HNMR (DMSO-d₆) δ 10.31 (s, 1H), 9.16 (s, 1H), 8.16 (m, 1H), 8.08 (d, 1H), 7.92 (m, 1H), 7.88 (d, 1H), 7.68 (m, 1H), 7.24-7.42 (m, 4H), 6.83 (d, 1H), 6.64 (m, 1H), 6.55 (d, 1H), 4.39 (m, 2H), 3.17 (m, 2H) and 1.05 ppm (m, 3H); LCMS: 470 [M+H]⁺, RT 2.77 min.

Examples 164 (using intermediate Q instead of intermediate P) and 165 (using intermediate R instead of intermediate P) were made using the method of example 162.

### Example 163

### Preparation of N-(2,2-difluoro-1,3-benzodioxol-5-yl)-2-({[2-({[(4-methoxyphenyl)amino]carbonyl}amino)pyridin-4-yl]methyl}amino)benzamide

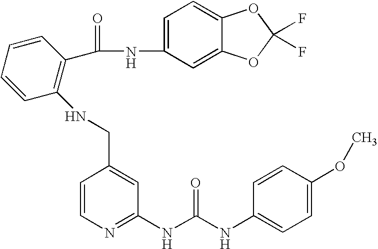

To a solution of 2-{[(2-aminopyridin-4-yl)methyl]amino}-N-(2,2-difluoro-1,3-benzodioxol-5-yl)benzamide (0.070 g, 0.176 mmol) (example 161) in CH₂Cl₂ (2 mL) was added 4-methoxyphenylisocyanate (0.027 g, 0.176 mmol) and diisopropylethylamine (0.2 mL). The resulting mixture was stirred at rt for 16 h under N₂. The white solid was collected by filtration and washed with CH₂Cl₂ and sequentially with diethyl ether. The resulting solid was dried under high vacuum to give N-(2,2-difluoro-1,3-benzodioxol-5-yl)-2-({[2-({[(4-methoxyphenyl)amino]carbonyl}amino)pyridin-4-yl]methyl}amino)benzamide as white solid (0.2 g, 21%).

¹H NMR (DMSO-d₆) δ 10.42 (s, 1H), 10.36 (s, 1H), 9.36 (s, 1H), 8.20 (d, 1H), 7.94 (m, 1H), 7.88 (d, 1H), 7.70 (dd, 1H), 7.24-7.48 (m, 4H), 6.98 (d, 1H), 6.92 (m, 2H), 6.62 (m, 1H), 6.58 (d, 1H), 4.42 (m, 2H) and 3.70 ppm (s, 3H); LC/MS 548.1[M+H]⁺, RT 3.28 min.

Examples 166-173 were made using the method of example 163 using the corresponding commercially available isocyanates rather than 4-methoxyphenylisocyanate.

| | | | | LCMS |
|---|---|---|---|---|
| Entry | | | LC-MS | RT |
| No. | Structure | Chemical Name | [M + H]+ | (min) |
| 164 | 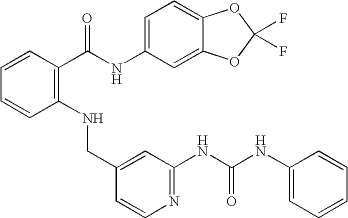 | 2-[({2-[(anilinocarbonyl)amino]pyridin-4-yl}methyl)amino]-N-(2,2-difluoro-1,3-benzodioxol-5-yl)benzamide; | 518.2 | 3.13 |
| 165 | 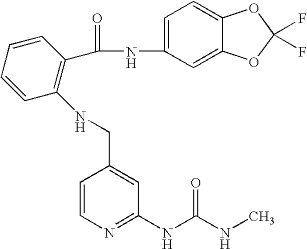 | N-(2,2-difluoro-1,3-benzodioxol-5-yl)-2-{[(2-{[(methylamino)carbonyl]amino}pyridin-4-yl)methyl]amino}benzamide; | 456.1 | 2.67 |
| 166 | 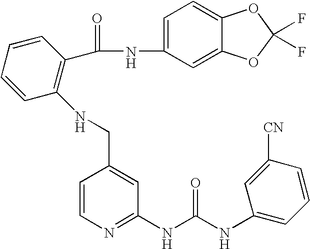 | 2-({[2-({[(3-cyanophenyl)amino]carbonyl}amino)pyridin-4-yl]methyl}amino)-N-(2,2-difluoro-1,3-benzodioxol-5-yl)benzamide; | 543 | 3.5 |
| 167 | 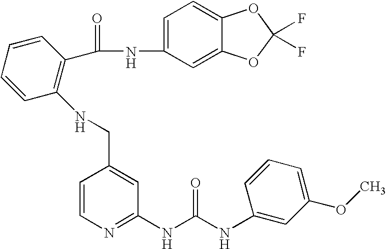 | N-(2,2-difluoro-1,3-benzodioxol-5-yl)-2-({[2-({[(3-methoxyphenyl)amino]carbonyl}amino)pyridin-4-yl]methyl}amino)benzamide; | 548.2 | 3.74 |
| 168 | 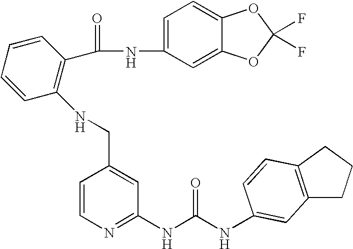 | N-(2,2-difluoro-1,3-benzodioxol-5-yl)-2-{[(2-{[(2,3-dihydro-1H-inden-5ylamino)carbonyl]amino}pyridin-4-yl)methyl]amino}benzamide; | 558.2 | 3.92 |
| 169 | 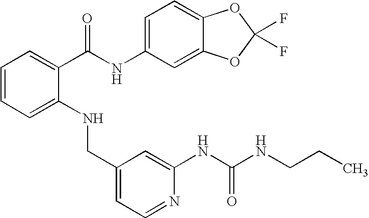 | N-(2,2-difluoro-1,3-benzodioxol-5-yl)-2-{[(2-{[(propylamino)carbonyl]amino}pyridin-4-yl)methyl]amino}benzamide; | 484.2 | 3.26 |
| 170 | 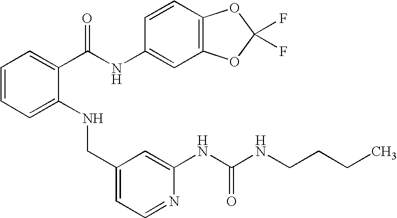 | 2-{[(2-{[(butylamino)carbonyl]amino}pyridin-4-yl)methyl]amino}-N-(2,2-difluoro-1,3-benzodioxol-5-yl)benzamide; | 498.2 | 3.38 |
| 171 | 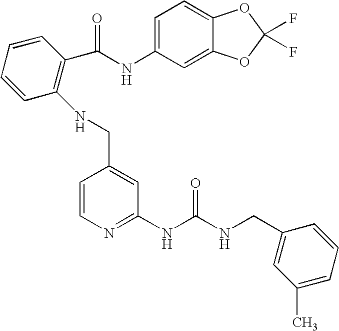 | N-(2,2-difluoro-1,3-benzodioxol-5-yl)-2-({[2-({[(3-methylbenzyl)amino]carbonyl}amino)pyridin-4-yl]methyl}amino)benzamide; | 546.2 | 3.58 |
| 172 | 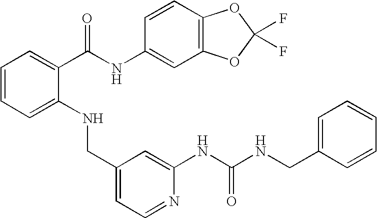 | 2-{[(2-{[(benzylamino)carbonyl]amino}pyridin-4-yl)methyl]amino]-N-(2,2-difluoro-1,3-benzodioxol-5-yl)benzamide; | 484.2 | 3.26 |
| 173 | 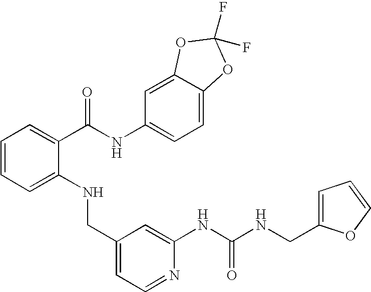 | N-(2,2-difluoro-1,3-benzodioxol-5-yl)-2-({[2-({[(2-furylmethyl)amino]carbonyl}amino)pyridin-4-yl]methyl}amino)benzamide; | 522.3 | 3.06 |

### Example 174

### Preparation of N-(2,2-difluoro-1,3-benzodioxol-5-yl)-2-{[(2-{[(dimethylamino)carbonyl]amino}pyridin-4-yl)methyl]amino}benzamide

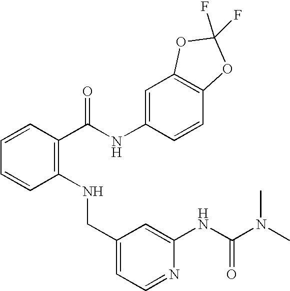

A solution of 2-{[(2-aminopyridin-4-yl)methyl]amino}-N-(2,2-difluoro-1,3-benzodioxol-5-yl)benzamide (example 161) (40.0 mg, 0.10 mmol) in 1,2-dichloroethane (1 mL) was treated with N,N-dimethylcarbamoyl chloride (0.010 mL, 0.10 mmol) and allowed to stir overnight at room temperature. The reaction mixture was quenched with methanol and saturated NaOH, then concentrated in vacuo. Purification of the crude residue by HPLC (10-90% MeCN in water containing 0.1% TFA gradient) gave 16.0 mg (34.1%) of the title compound. N,N-dimethylurea protons do not show up in ¹H NMR.

¹H NMR (300 MHz, CD₃CN) δ 8.85 (bs, 1H), 7.68-7.80 (m, 3H), 7.28-7.38 (m, 2H), 7.20 (d, 1H), 6.89 (s, 1H), 6.70-6.82 (m, 2H), 6.55 (d, 1H), 4.48 (s, 2H), 2 CH₃'s are not seen in NMR; ES-MS m/z 470.1 [M+H]⁺, HPLC RT (min) 2.57.

### Example 175

### Preparation of N-(2,2-difluoro-1,3-benzodioxol-5-yl)-2-[({2-[(methylsulfonyl)amino]pyridin-4-yl}methyl)amino]benzamide

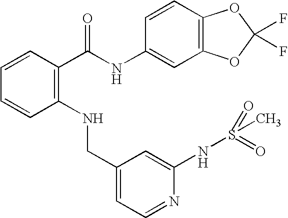

### Step 1: Preparation of 2-[({2-[bis(methylsulfonyl)amino]pyridin-4-yl}methyl)amino]-N-(2,2-difluoro-1,3-benzodioxol-5-yl)benzamide

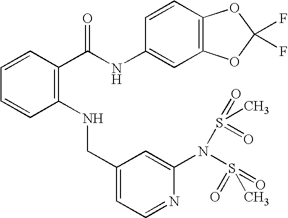

A solution of 2-amino-N-(2,2-difluoro-1,3-benzodioxol-5-yl)benzamide (Intermediate W) (100 mg, 0.34 mmol) and N-[4-(chloromethyl)pyridin-2-yl]-N-(methylsulfonyl)methanesulfonamide (benzamide (Intermediate 0 step 1) (112 g, 0.38 mmol) in dry DMF (1.5 mL) added sodium iodide (77 mg, 0.51 mmol). The resulting mixture was heated with stirring at 60° C. for 16 h. The reaction was cooled and diluted with ethylacetate. The organic layer was extracted with water (3×), dried with sodium sulfate and evaporated under vacuum. The residue was purified by HPLC to obtain 2-[({2-[bis(methylsulfonyl)amino]pyridin-4-yl}methyl)amino]-N-(2,2-difluoro-1,3-benzodioxol-5-yl)benzamide (105 mg, 55%).

¹H NMR (300 MHz, CD₃OD-d₄) δ 8.45 (d, 1H), 7.75 (bs, 1H), 7.69 (d, 1H), 7.53 (m, 2H), 7.38-7.24 (m, 2H), 7.18 (d, 1H), 6.74 (t, 1H), 6.58 (d, 1H), 4.60 (s, 2H), 3.52 (s, 6H); ES-MS m/z 577.0 [M+Na]⁺, HPLC RT (min) 3.15.

### Step 2: Preparation of N-(2,2-difluoro-1,3-benzodioxol-5-yl)-2-[({2-[(methylsulfonyl)amino]pyridin-4-yl}methyl)amino]benzamide

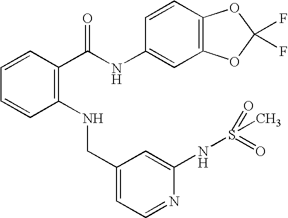

A solution of 2-[({2-[bis(methylsulfonyl)amino]pyridin-4-yl}methyl)amino]-N-(2,2-difluoro-1,3-benzodioxol-5-yl)benzamide (70 mg, 0.13 mmol) in MeOH (1 mL) was added 1N aqueous sodium hydroxide (0.63 mL, 0.63 mmol). The resulting mixture was stirred at rt for 1 h. 2N HCl was added until the pH was between 6 and 3. The white solid that crashed out of the solution was filtered and washed with MeOH to obtain N-(2,2-difluoro-1,3-benzodioxol-5-yl)-2-[({2-[(methylsulfonyl)amino]pyridin-4-yl}methyl)amino]benzamide (35 mg, 58%).

¹H NMR (300 MHz, DMSO-d₆) δ 10.32 (s, 1H), 8.12 (bm, 1H), 7.93-7.86 (m, 2H), 7.72 (d 1H), 7.46-7.39 (m, 2H), 7.18 (t, 1H), 6.92 (bm, 2H), 6.65 (t, 1H); 6.52 (d, 1H); 4.42 (d, 2H) and 3.20 ppm (bs, 3H); ES-MS m/z 477.0 [M+H]⁺, HPLC RT (min) 2.97.

### Example 176

### Preparation of N-(2,2-difluoro-1,3-benzodioxol-5-yl)-2-[({2-[(4-methyl-1,3-thiazol-2-yl)amino]pyridin-4-yl}methyl)amino]benzamide

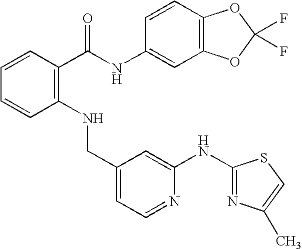

A solution of 2-amino-N-(2,2-difluoro-1,3-benzodioxol-5-yl)benzamide (Intermediate W) (356 mg, 1.22 mmol) and 4-(chloromethyl)-N-(4-methyl-1,3-thiazol-2-yl)pyridin-2-amine (Intermediate U) (225 g, 0.94 mmol) in dry DMF (1 mL) added sodium iodide (211 mg, 1.41 mmol). The resulting mixture was heated with stirring at 60 C for 16 h. The reaction was cooled and evaporated under vacuum. The residue was purified by chromatography on silica gel using 0-100% ethyl acetate in hexane to obtain N-(2,2-difluoro-1,3-benzodioxol-5-yl)-2-[({2-[(4-methyl-1,3-thiazol-2-yl)amino]pyridin-4-yl}methyl)amino]benzamide (130 mg, 28%) as a white solid.

¹H NMR (300 MHz, DMSO-d₆) δ 11.13 (s, 1H), 8.20 (d, 1H), 7.97 (m, 1H), 7.91 (m, 1H), 7.74 (m, 1H), 7.50 (m, 1H), 7.40 (m, 1H), 7.29 (m, 1H), 7.00 (s, 1H), 6.87 (m, 1H), 6.68 (m, 1H), 6.54 (m, 2H), 4.45 (bs, 2H), 2.22 (s, 3H); ES-MS m/z 496.1 [M+H]⁺, HPLC RT (min) 3.07.

### Example 177

### Preparation of N-(2,2-difluoro-1,3-benzodioxol-5-yl)-2-({[2-(hydroxymethyl)pyridin-4-yl]methyl}amino)benzamide

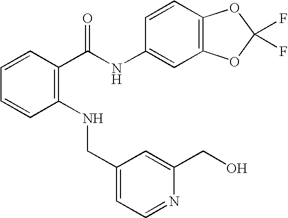

To a flask containing methyl 4-{[(2-{[(2,2-difluoro-1,3-benzodioxol-5-yl)amino]-carbonyl}phenyl)amino]methyl}pyridine-2-carboxylate (Example 76) (90 mg, 0.20 mmol) in THF (3 mL) slowly added LiBH₄ (0.15 mL, 0.31 mmol) and drop of MeOH and the reaction was stirred overnight at room temperature. The reaction was quenched with water and adjusted the pH to 6-7 by addition of 2N HCl. The aqueous layer was extracted with ethylacetate. The organic layer was separated and washed with Sat. sodium bicarbonate, dried over sodium sulfate and concentrated under vacuum. The residue was purified by flash chromatography to afford N-(2,2-difluoro-1,3-benzodioxol-5-yl)-2-({[2-(hydroxymethyl)-pyridin-4-yl]methyl}amino)benzamide (45 mg, 53%).

¹H NMR (300 MHz, CD₃OD-d₄) δ 8.53 (d, 1H), 7.94 (m, 1H), 7.76 (m, 1H), 7.67 (m, 1H), 7.44 (m, 1H), 7.34-7.31 (m, 1H), 7.24 (t, 1H), 7.17-7.14 (m, 1H), 6.70 (t, 1H), 6.50 (m, 1H), 4.89 (s, 2H), 4.64 (s, 2H); ES-MS m/z 414.2 [M+H]⁺, HPLC RT (min) 2.89.

### Example 177a

### Preparation of N-(4-{[(2-{[(2,2-difluoro-1,3-benzodioxol-5-yl)amino]carbonyl}phenyl)amino]methyl}pyridin-2-yl)morpholine-4-carboxamide

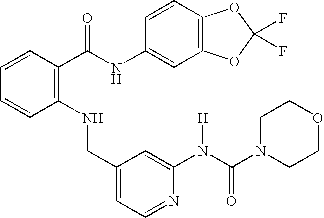

### Step 1: Preparation of N-[4-(chloromethyl)pyridin-2-yl]morpholine-4-carboxamide

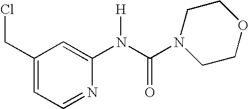

N-[4-(chloromethyl)pyridin-2-yl]morpholine-4-carboxamide can be prepared using the following method. A suspension of 4-(chloromethyl)pyridin-2-amine (from step 1 of intermediate I) and triethylamine in dichloroethane can be stirred under nitrogen with ice bath cooling as 4-morpholinocarbonyl chloride is added slowly over 10 min. After stirring for ˜2 h, following disappearance of starting material using TLC. The mixture can be diluted with dichloromethane and washed with water and then brine. The solution could be dried (Na₂SO₄) and evaporated in vacuo. The residue can be purified by chromatography on silica gel using a gradient from ˜0-3% methanol in dichloromethane to yield the pure title compound.

### Step 2: Preparation of the Title Compound

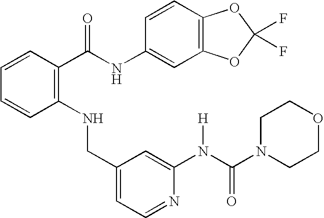

The title compound can be prepared using the following method. Sodium Iodide can be added to a solution of 2-amino-N-(2,2-difluoro-1,3-benzodioxol-5-yl)benzamide (Intermediate W) and N-[4-(chloromethyl)pyridin-2-yl]morpholine-4-carboxamide (step 1 above) in dry DMF. The resulting mixture can be heated with stirring at 60° C. for 16 h. The reaction can be cooled and diluted with ethylacetate. The organic layer extracted with water, dried with sodium sulfate and evaporated under vacuum. The residue can be purified by HPLC to obtain the title compound.

### Example 178

### Preparation of N-(4-{[(2-{[(2,2-difluoro-1,3-benzodioxol-5-yl)amino]carbonyl}phenyl)amino]methyl}pyridin-2-yl)pyrrolidine-1-carboxamide

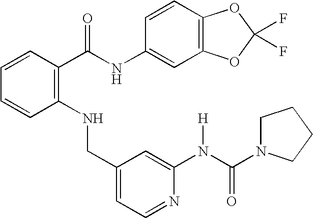

The title compound can be prepared using the same procedure as example 177 except using 1-pyrrolidinecarbonyl chloride as a substitute for 4-morpholinocarbonyl chloride.

Generally, a desired salt of a compound of this invention can be prepared in situ during the final isolation and purification of a compound by means well known in the art. Or, a desired salt can be prepared by separately reacting the purified compound in its free base form with a suitable organic or inorganic acid and isolating the salt thus formed. These methods are conventional and would be readily apparent to one skilled in the art.

Additionally, sensitive or reactive groups on the compound of this invention may need to be protected and deprotected during any of the above methods. Protecting groups in general may be added and removed by conventional methods well known in the art (see, for example, T. W. Greene and P. G. M. Wuts, Protective Groups in Organic Synthesis; Wiley: New York, (1999).

Compositions of the Compounds of this Invention

The compounds of this invention can be utilized to achieve the desired pharmacological effect by administration to a patient in need thereof in an appropriately formulated pharmaceutical composition. The present invention includes pharmaceutical compositions that are comprised of a pharmaceutically acceptable carrier and a pharmaceutically effective amount of a compound, or salt, solvate or solvate of the salt thereof, of the present invention. A pharmaceutically acceptable carrier is any carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, otically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quaternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

It is believed that one skilled in the art, using the preceding information, can utilize the present invention to its fullest extent. Nevertheless, the following are examples of pharmaceutical formulations that can be used in the composition of the present invention. They are for illustrative purposes only, and are not to be construed as limiting the invention in any way.

Pharmaceutical compositions according to the present invention can be illustrated as follows:

Sterile IV Solution: A 2 mg/mL solution of the desired compound of this invention is made using sterile, injectable water, and the pH is adjusted if necessary. The solution is diluted for administration to 0.2-1 mg/mL with sterile 5% dextrose and is administered as an IV infusion over 120 minutes.

Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-120 min.

Intramuscular suspension: The following solution or suspension can be prepared, for intramuscular injection:
50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Method of Treating

Another embodiment of the present invention relates to a method of using the compounds described above, including salts and pro-drugs thereof and corresponding compositions thereof, as cancer chemotherapeutic agents. This method comprises administering to a patient an amount of a compound of this invention, or a pharmaceutically acceptable salt thereof, which is effective to treat the patient's cancer. A patient, for the purpose of this invention, is a mammal, including a human, in need of treatment for a particular cancer. Cancers include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The utility of the compounds of the present invention can be illustrated, for example, by their activity in the PAKT/PKB Cytoblot Assay described below.

The involvement of the AKT/PKB[PI3K/AKt] pathway as a target for cancer chemotherapy has been recognized in the art. For example, see F. Chang et al, Involvement of PI3K/Akt pathway in cell cycle progression, apoptosis, and neoplastic transformation: a target for cancer chemotherapy, Leukemia, 2003, 17: p. 590-603; K. A. West et al, Activation of the PI3K/Akt pathway and chemotherapeutic resistance, Drug Resistance Updates, 2002, 5: p. 234-248; and P. Sen et al, Involvement of the Akt/PKB signaling pathway with disease processes, Molecular and Cellular Biochemistry, 2003, 253: p. 241-246.

The following assay is one of the methods by which compound activity relating to treatment of the disorders identified herein can be determined.

PAKT/PKB Cytoblot Assay Protocol with H209 Cells

H209 small cell lung carcinoma cells in log phase were plated at 50,000 cells/well in 96-well poly-lysine coated, clear bottom/black-sided plates (BD Cat # 354640) in 100 μl RPMI medium containing 0.1% (w/v) BSA, and incubated overnight at 37° C. in 5% CO₂ incubator. The following day, compounds (10 mM stock solutions in DMSO) were added to the plates to generate final concentrations of 0.0, 0.01, 0.03, 0.1, 0.3, 1.0, 3.0 and 10 μM for IC₅₀ determinations and incubated for 1 hour at 37° C. Cells were then left untreated or stimulated with Stem Cell Factor (SCF: Biosource Cat # PHC2116) at a final concentration of 25 ng/mL for 5 minutes at 37° C. in 5% CO₂ incubator. The media was then removed using a vacuum manifold and the cells were washed once with Tris Buffered Saline (TBS).

Cells were then fixed by adding 200 μl of cold 3.7% (v/v) formaldehyde in TBS to each well for 15 minutes at 4° C. After removal of the formaldehyde, the cells were treated with the addition of 50 μl of methanol (at −20° C.) to each well for 5 minutes. After removal of the methanol, 2001 of 1% (w/v) BSA in TBS was added to each well to block non-specific antibody binding sites and the plate was incubated at room temperature for 30 minutes.

After removal of the blocking buffer, 50 μl of p-(S473) AKT rabbit polyclonal antibody (Cell Signaling Cat # 9277S) was added at a dilution of 1:250 in 0.1% (w/v) BSA in TBS, and the plate was incubated at room temperature for 1 hour. Plates were then washed 3 times with cold TBS containing 0.05% (v/v) Tween 20 (TBS-T) and 1001 of Horseradish peroxidase (HRP)-conjugated goat-anti-rabbit antibody (Amersham Cat # NA934V) at a dilution of 1:250 in TBS-T was added and the plate was incubated at room temperature for 1 h. After washing with ice-cold TBS-T four times, 100 μl of Enhanced Chemiluminescence (ECL) reagent (Amersham Cat# RPN2209) was added to each well and mixed on a mini-orbital shaker for 1 min. The plate was then read on a Perkin Elmer Victor 5 Multilabel Counter (#1420-0421).

Compounds of the invention were tested in the above PAKT/PKB Cytoblot assay, with the result that examples 2-6, 9-15, 17-25, 29-34, 37, 44-49, 51, 74, 75, 76, 79, 82, 83, 85, 86, 87, 88, 89, 91, 92, 95, 96, 97, 100, 103, 104, 110, 111, 113, 114, 117, 119, 120, 123, 124, 132, 133, 134, 135, 136, 137, 138, 139, 140, 141, 143, 144, 145, 146, 147, 148, 149, 151, 152, 153, 155, 156, 157, 158, 159, 160, 162, 165, 169, 170, 173, 174, 175, and 176 exhibited IC₅₀ values of less than 500 nM. Examples 8, 16, 28, 38, 40, 43, 50, 78, 80, 84, 90, 94, 98, 105, 108, 112, 129, 142, 150, 154, 163, 172, and 177 exhibited IC₅₀ values between 500 nM and 1 μM. Examples 1, 7, 26, 27, 36, 39, 41, 42, 93, 99, 102, 106, 107, 109, 116, 118, 121, 122, 125, 126, 127, 128, 166, 167, and 171 exhibited IC₅₀ values between 1 and 3 μM.

Based upon the above and other standard laboratory techniques known to evaluate compounds useful for the treatment of cancers, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.01 mg/kg to about 200 mg/kg, and preferably from about 0.1 mg/kg to about 20 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or composition thereof can be ascertained by those skilled in the art using conventional treatment tests.

The compounds of this invention can be administered as the sole pharmaceutical agent or m combination with one or more other pharmaceutical agents where the combination causes no unacceptable adverse effects. For example, the compounds of this invention can be combined with known anti-hyper-proliferative, chemotherapeutic, or other indication agents, and the like, as well as with admixtures and combinations thereof.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with this invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine. Other anti-hyper-proliferative agents suitable for use with this invention include but are not limited to other anti-cancer agents such as epothilone, irinotecan, raloxifen and topotecan.

It is believed that one skilled in the art, using the preceding information, can utilize the present invention to its fullest extent.

It should be apparent to one of ordinary skill in the art that changes and modifications can be made to this invention without departing from the spirit or scope of the invention as it is set forth herein.

We claim:

1. A compound having the formula (1) 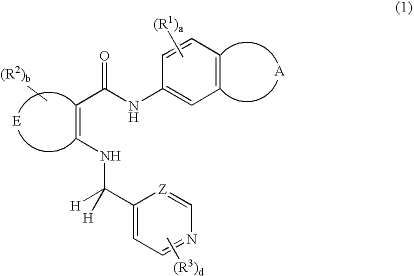
wherein
E represents 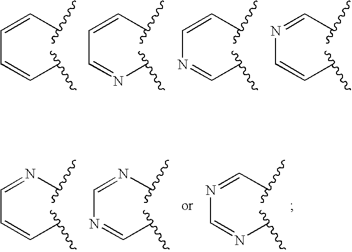
Z represents CH or N when E is 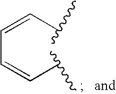
Z represents CH when E is 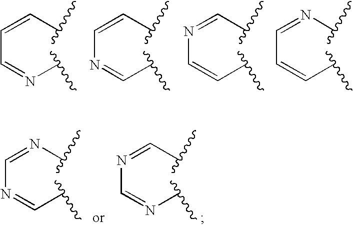
R¹ represents C₁₋₄ alkyl or halogen;
a represents 0, 1 or 2;
R² represents C₁₋₄ alkyl, C₁₋₄ alkoxy, or halogen;
b represents 0, 1, 2, or 3 when E is 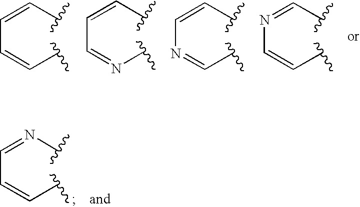
b represents 0 or 1 when E is 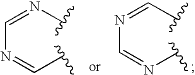
with the proviso that when b is 1 and E is 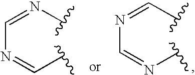
then the group R² is located adjacent to the amino or amido moiety, respectively, of formula (1);
R³ represents
1) 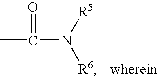
R⁵ represents H or C₁₋₄ alkyl which may optionally be substituted by OH or C₁₋₄ alkoxy; and
R⁶ represents
H;
C₃₋₆ cycloalkyl;
—(CH₂)_{f}—O—(CH₂)_{g}—OR¹⁰ wherein R¹⁰ represents H or C₁₋₄ alkyl which is optionally substituted by F, and the subscripts f and g each independently represents 1, 2, or 3; 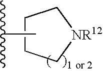
wherein the ring is optionally substituted by C₁₋₄ alkyl, and wherein R¹² represents H; C₁₋₄ alkyl which is optionally substituted by F; phenyl; benzyl; C₁₋₄ acyl; or SO₂R^{12a} in which R^{12a} represents C₁₋₄ alkyl which is optionally substituted by F; or
C₁₋₆ alkyl, which is optionally fluorinated up to perfluoro, or independently substituted by up to three substituents selected from:
—SO₂R¹⁴ in which R¹⁴ represents C₁₋₅ alkyl which is optionally substituted by C₁₋₄ alkoxy or F; 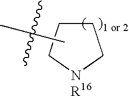
wherein the ring is optionally substituted by C₁₋₄ alkyl; and wherein R¹⁶ represents H; C₁₋₄ alkyl optionally substituted by F; phenyl; benzyl; C₁₋₄ acyl; or SO₂R^{16a} in which R^{16a} represents C₁₋₄ alkyl optionally substituted by F; 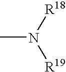
wherein R¹⁸ and R¹⁹ each independently represents H; C₁₋₅ alkyl; or phenyl which is optionally substituted by C₁₋₄ alkyl, halogen, OH, C₁₋₄ alkoxy, C₁₋₄ acyl, or SO₂R^{19a} in which R^{19a} represents C₁₋₄ alkyl optionally substituted by F; 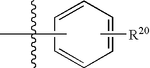
wherein R²⁰ represents from 0 to 4 optional substituents independently selected from C₁₋₄ alkyl, OH, C₁₋₄ alkoxy, halogen, NO₂, CN, and morpholino; 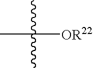
wherein R²² represents H or C₁₋₅ alkyl which is optionally substituted by F, 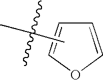
which is optionally substituted by C₁₋₄ alkyl; 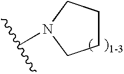
which is optionally substituted by C₁₋₄ alkoxy, F, or C₁₋₄ alkyl which is optionally substituted by F; 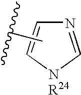
in which the ring is optionally substituted by C₁₋₄ alkyl or halogen, and R²⁴ represents H or C₁₋₄ alkyl which is optionally substituted by F; 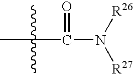
wherein R²⁶ and R²⁷ independently represent H or C₁₋₂ alkyl optionally substituted by F; 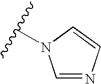
which is optionally substituted by halogen or by C₁₋₄ alkyl optionally substituted by F;
—CO₂R²⁸ wherein R²⁸ represents C₁₋₄ alkyl; 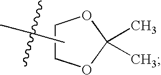 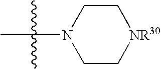
wherein the ring is optionally substituted by C₁₋₄ alkyl, C₁₋₄ alkoxy, or F; and R³⁰ represents H; C₁₋₄ alkyl optionally substituted by F; phenyl; C₁₋₄ acyl; or SO₂R³¹ wherein R³¹ represents C₁₋₂ alkyl optionally substituted by F; 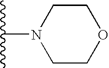
optionally substituted by F or C₁₋₄ alkyl; 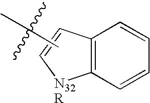
wherein R³² represents H or C₁₋₄ alkyl optionally substituted by F, and the phenyl ring is optionally substituted by C₁₋₄ alkyl, C₁₋₄ alkoxy, or halogen; 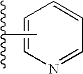
which is optionally substituted by C₁₋₄ alkyl or C₁₋₄ alkoxy; 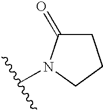
which is optionally substituted by C₁₋₄ alkyl, C₁₋₄ alkoxy, or F;
—SR³⁴ wherein R³⁴ represents H or C₁₋₄ alkyl; 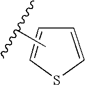
which is optionally substituted by C₁₋₄ alkyl or halogen; 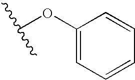
which is optionally substituted by halogen, OH, C₁₋₄ alkoxy, or C₁₋₄ alkyl which is optionally substituted by F; 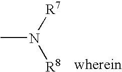
2)
R⁷ represents H or C₁₋₄ alkyl;
R⁸ represents
H;
—(CH₂)ₕ—O—(CH₂)ᵢ—OR³⁶ wherein
R³⁶ represents H or C₁₋₄ alkyl which is optionally substituted by F, and
subscripts h and i are independently 1, 2, or 3; 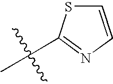
which is optionally substituted by halogen or by C₁₋₄ alkyl which is optionally substituted by F; or
C₁₋₆ alkyl which is optionally substituted by up to three substituents selected from:
OH;
C₁₋₄ alkoxy; 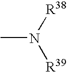
wherein R³⁸ and R³⁹ independently represent H, C₁₋₄ alkyl, C₁₋₄ acyl, or SO₂R^{39a}, and R^{39a} represents C₁₋₄ alkyl optionally substituted by F; 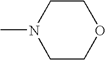
which is optionally substituted by F or C₁₋₄ alkyl; 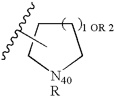
wherein the ring is optionally substituted by F, C₁₋₄ alkyl, or C₁₋₄ alkoxy; and R⁴⁰ represents H, C₁₋₄ acyl, C₁₋₄ alkyl which is optionally substituted by F, or SO₂R^{40a} wherein R^{40a} represents C₁₋₄ alkyl which is optionally substituted by F; 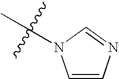
which is optionally substituted by halogen or C₁₋₄ alkyl which is optionally substituted by F; 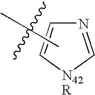
wherein the ring is optionally substituted by halogen, and R⁴² represents H or C₁₋₄ alkyl which is optionally substituted by F; 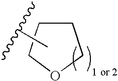
which is optionally substituted by F or C₁₋₄ alkyl; and 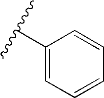
which is optionally substituted by halogen, C₁₋₄ alkoxy, or C₁₋₄ alkyl which is optionally substituted by F;
3) —CN;
4) -halogen;
5)-C₁₋₄ alkyl which is optionally substituted by OH or C₁₋₄ alkoxy; 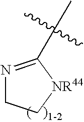
6) wherein the ring is optionally substituted by F, C₁₋₄ alkoxy, or C₁₋₄ alkyl, and R⁴⁴ represents H or C₁₋₄ alkyl optionally substituted by F;
7) 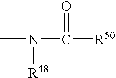
wherein
R⁴⁸, represents H, C₁₋₂ alkyl, or C(O)—(CH₂)₁₋₃—CO₂R^{48a} wherein R^{48a} represents H or C₁₋₄ alkyl; and
R⁵⁰ represents
—(CH₂)ⱼ—O—(CH₂)ₖ—R^{50a} wherein
R^{50a} represents OH, C₁₋₄ alkoxy, or C₁₋₄ alkoxycarbonyl; and
subscripts j and k are independently 1, 2, or 3;
or R⁵⁰ represents
C₁₋₄ alkyl optionally substituted by C₁₋₂ alkoxy, C₁₋₄ acyloxy, or C₁₋₄ alkoxycarbonyl;
8) 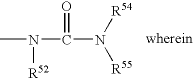
R⁵² represents H or C₁₋₂ alkyl;
R⁵⁴ represents H or C₁₋₄ alkyl; and
R⁵⁵ represents H; C₁₋₄ alkyl optionally substituted by F or C₁₋₄ alkoxy; phenyl optionally substituted by CN, OH, C₁₋₄ alkoxy, or C₁₋₄ alkyl; 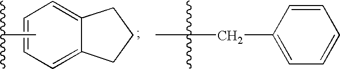
wherein the ring is optionally substituted by C₁₋₄ alkyl, C₁₋₄ alkoxy, or OH; or R⁵⁵ represents 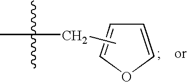
R⁵⁴ and R⁵⁵ are optionally joined and together with the N atom to which they are attached form a 5- or 6-membered saturated heterocycle selected from pyrrolidinyl, morpholinyl, thiomorpholinyl, and piperizinyl optionally substituted on N with C₁₋₄ alkyl; 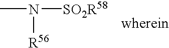
9)
R⁵⁶ represents H or C₁₋₂ alkyl; and
R⁵⁸ represents C₁₋₄ alkyl which is optionally substituted by F; or represents phenyl which is optionally substituted by halogen, C₁₋₄ alkyl, or C₁₋₄ alkoxy;
d is 0 or 1;
A represents 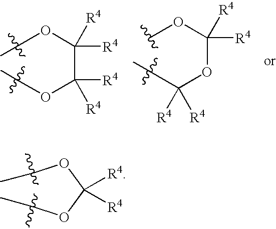
R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring;
and with the further proviso that any R³ group is located adjacent to a ring nitrogen atom;
or a pharmaceutically acceptable salt or stereoisomer thereof.

2. A compound having the formula (I) 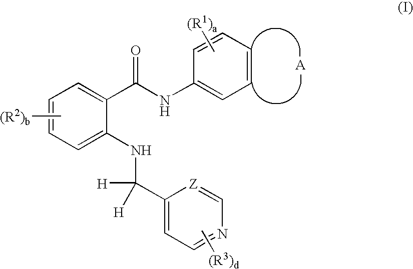
wherein
Z represents CH or N;
R¹ represents C₁₋₄ alkyl or halogen;
a represents 0, 1 or 2;
R² represents C₁₋₄ alkyl, C₁₋₄ alkoxy, or halogen;
b represents 0, 1, 2, or 3;
R³ represents
1) 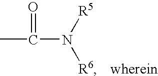
R⁵ represents H or C₁₋₄ alkyl which may optionally be substituted by OH or C₁₋₄ alkoxy; and
R⁶ represents
H;
C₃₋₆ cycloalkyl;
—(CH₂)_{f}—O—(CH₂)_{g}—OR¹⁰ wherein R¹⁰ represents H or C₁₋₄ alkyl which is optionally substituted by F, and the subscripts f and g each independently represents 1, 2, or 3; 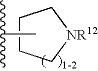
wherein the ring is optionally substituted by C₁₋₄ alkyl, and wherein R¹² represents H; C₁₋₄ alkyl which is optionally substituted by F; phenyl; benzyl; C₁₋₄ acyl; or SO₂R^{12a} in which R^{12a} represents C₁₋₄ alkyl which is optionally substituted by F; or
C₁₋₆ alkyl, which is optionally fluorinated up to perfluoro, or independently substituted by up to three substituents selected from:
—SO₂R¹⁴ in which R¹⁴ represents C₁₋₅ alkyl which is optionally substituted by C₁₋₄ alkoxy or F; 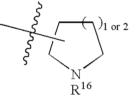
wherein the ring is optionally substituted by C₁₋₄ alkyl; and wherein R¹⁶ represents H; C₁₋₄ alkyl optionally substituted by F; phenyl; benzyl; C₁₋₄ acyl; or SO₂R^{16a} in which R^{16a} represents C₁₋₄ alkyl optionally substituted by F; 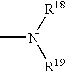
wherein R¹⁸ and R¹⁹ each independently represents H; C₁₋₅ alkyl; or phenyl which is optionally substituted by C₁₋₄ alkyl, halogen, OH, C₁₋₄ alkoxy, C₁₋₄ acyl, or SO₂R^{19a} in which R^{19a} represents C₁₋₄ alkyl optionally substituted by F; 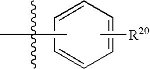
wherein R²⁰ represents from 0 to 4 optional substituents independently selected from C₁₋₄ alkyl, OH, C₁₋₄ alkoxy, halogen, NO₂, CN, and morpholino; 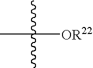
wherein R²² represents H or C₁₋₅ alkyl which is optionally substituted by F, 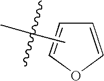
which is optionally substituted by C₁₋₄ alkyl; 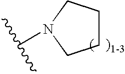
which is optionally substituted by C₁₋₄ alkoxy, F, or C₁₋₄ alkyl which is optionally substituted by F; 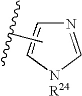
in which the ring is optionally substituted by C₁₋₄ alkyl or halogen, and R²⁴ represents H or C₁₋₄ alkyl which is optionally substituted by F; 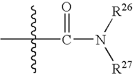
wherein R²⁶ and R²⁷ independently represent H or C₁₋₂ alkyl optionally substituted by F; 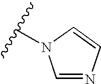
which is optionally substituted by halogen or by C₁₋₄ alkyl optionally substituted by F;
—CO₂R²⁸ wherein R²⁸ represents C₁₋₄ alkyl; 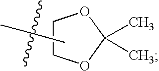 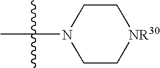
wherein the ring is optionally substituted by C₁₋₄ alkyl, C₁₋₄ alkoxy, or F; and wherein R³⁰ represents H; C₁₋₄ alkyl optionally substituted by F; phenyl; C₁₋₄ acyl; or SO₂R³¹ wherein R³¹ represents C₁₋₂ alkyl optionally substituted by F; 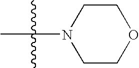
optionally substituted by F or C₁₋₄ alkyl; 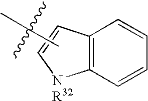
wherein R³² represents H or C₁₋₄ alkyl optionally substituted by F, and the phenyl ring is optionally substituted by C₁₋₄ alkyl, C₁₋₄ alkoxy, or halogen; 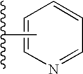
which is optionally substituted by C₁₋₄ alkyl or C₁₋₄ alkoxy; 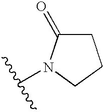
which is optionally substituted by C₁₋₄ alkyl, C₁₋₄ alkoxy, or F;
—SR³⁴ wherein R³⁴ represents H or C₁₋₄ alkyl; 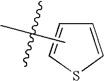
which is optionally substituted by C₁₋₄ alkyl or halogen; 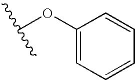
which is optionally substituted by halogen, OH, C₁₋₄ alkoxy, or C₁₋₄ alkyl which is optionally substituted by F;
2) 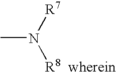
R⁷ represents H or C₁₋₄ alkyl;
R⁸ represents
H;
-(CH₂)ₕ—O—(CH₂)ᵢ—OR³⁶ wherein
R³⁶ represents H or C₁₋₄ alkyl which is optionally substituted by F, and
subscripts h and i are independently 1, 2, or 3; 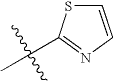
which is optionally substituted by halogen or by C₁₋₄ alkyl which is optionally substituted by F; or
C₁₋₆ alkyl which is optionally substituted by up to three substituents selected from:
OH;
C₁₋₄ alkoxy; 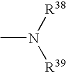
wherein R³⁸ and R³⁹ independently represent H, C₁₋₄ alkyl, C₁₋₄ acyl, or SO₂R^{39a}, and R^{39a} represents C₁₋₄ alkyl optionally substituted by F; 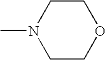
which is optionally substituted by F or C₁₋₄ alkyl; 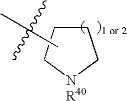
wherein the ring is optionally substituted by F, C₁₋₄ alkyl, or C₁₋₄ alkoxy; and R⁴⁰ represents H, C₁₋₄ acyl, C₁₋₄ alkyl which is optionally substituted by F, or SO₂R^{40a} wherein R^{40a} represents C₁₋₄ alkyl which is optionally substituted by F; 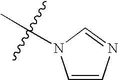
which is optionally substituted by halogen or C₁₋₄ alkyl which is optionally substituted by F; 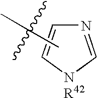
wherein the ring is optionally substituted by halogen, and R⁴² represents H or C₁₋₄ alkyl which is optionally substituted by F; 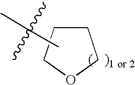
which is optionally substituted by F or C₁₋₄ alkyl; and 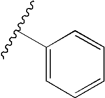
which is optionally substituted by halogen, C₁₋₄ alkoxy, or C₁₋₄ alkyl which is optionally substituted by F;
3) —CN;
4) -halogen;
5) —C₁₋₄ alkyl which is optionally substituted by OH or C₁₋₄ alkoxy;
6) 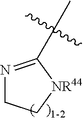
wherein the ring is optionally substituted by F, C₁₋₄ alkoxy, or C₁₋₄ alkyl, and R⁴⁴ represents H or C₁₋₄ alkyl optionally substituted by F;
7) 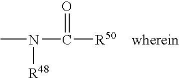
R⁴⁸ represents H, C₁₋₂ alkyl, or C(O)—(CH₂)₁₋₃—CO₂R^{48a} wherein R^{48a} represents H or C₁₋₄ alkyl; and
R⁵⁰ represents
—(CH₂)ⱼ—O—(CH₂)ₖ—R^{50a} wherein
R^{50a} represents OH, C₁₋₄ alkoxy, or C₁₋₄ alkoxycarbonyl; and subscripts j and k are independently 1, 2, or 3;
or R⁵⁰ represents
C₁₋₄ alkyl optionally substituted by C₁₋₂ alkoxy, C₁₋₄ acyloxy, or C₁₋₄ alkoxycarbonyl; 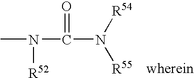
8)
R⁵² represents H or C₁₋₂ alkyl;
R⁵⁴ represents H or C₁₋₄ alkyl; and
R⁵⁴ represents H; C₁₋₄ alkyl optionally substituted by F or C₁₋₄ alkoxy; phenyl optionally substituted by CN, OH, C₁₋₄ alkoxy, or C₁₋₄ alkyl; 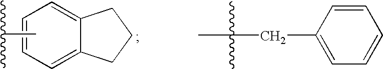
wherein the ring is optionally substituted by C₁₋₄ alkyl, C₁₋₄ alkoxy, or OH; or R⁵⁵ represents 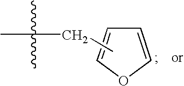
R⁵⁴ and R⁵⁵ are optionally joined and together with the N atom to which they are attached form a 5- or 6-membered saturated heterocycle selected from pyrrolidinyl, morpholinyl, thiomorpholinyl, and piperizinyl optionally substituted on N with C₁₋₄ alkyl; 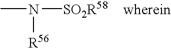
9)
R⁵⁶ represents H or C₁₋₂ alkyl; and
R⁵⁸ represents C₁₋₄ alkyl which is optionally substituted by F; or represents phenyl which is optionally substituted by halogen, C₁₋₄ alkyl, or C₁₋₄ alkoxy;
d is 0 or 1;
A represents 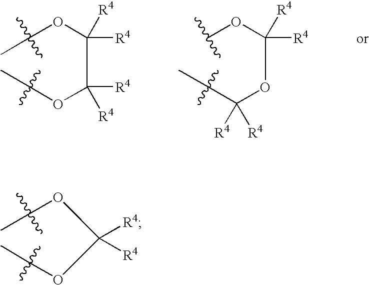
R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring;
and with the further proviso that any R³ group is located adjacent to a ring nitrogen atom;
or a pharmaceutically acceptable salt or stereoisomer thereof.

3. The compound of claim 2 wherein
Z represents CH;
R³ represents
1) 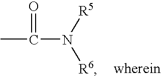
R⁵ represents H or C₁₋₄ alkyl which may optionally be substituted by OH or C₁₋₄ alkoxy; and
R⁶ represents
H;
C₃₋₆ cycloalkyl;
—(CH₂)_{f}—O—(CH₂)_{g}—OR¹⁰ wherein R¹⁰ represents H or C₁₋₄ alkyl which is optionally substituted by F, and the subscripts f and g each independently represents 1, 2, or 3;
C₁₋₆ alkyl, which is optionally fluorinated up to perfluoro, or independently substituted by up to three substituents selected from:
—SO₂R¹⁴ in which R¹⁴ represents C₁₋₅ alkyl which is optionally substituted by C₁₋₄ alkoxy or F; 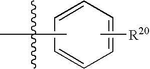
wherein R²⁰ represents from 0 to 4 optional substituents independently selected from C₁₋₄ alkyl, OH, C₁₋₄ alkoxy, halogen, NO₂, CN, and morpholino; 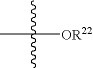
wherein R²² represents H or C₁₋₅ alkyl which is optionally substituted by F, 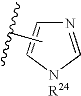
in which the ring is optionally substituted by C₁₋₄ alkyl or halogen, and R²⁴ represents H or C₁₋₄ alkyl which is optionally substituted by F; 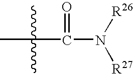
wherein R²⁶ and R²⁷ independently represent H or C₁₋₂ alkyl optionally substituted by F; 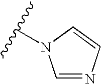
which is optionally substituted by halogen or by C₁₋₄ alkyl optionally substituted by F;
—CO₂R²⁸ wherein R²⁸ represents C₁₋₄ alkyl; 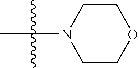
optionally substituted by F or C₁₋₄ alkyl; 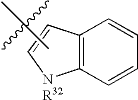
wherein R³² represents H or C₁₋₄ alkyl optionally substituted by F, and the phenyl ring is optionally substituted by C₁₋₄ alkyl, C₁₋₄ alkoxy, or halogen; 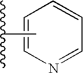
which is optionally substituted by C₁₋₄ alkyl or C₁₋₄ alkoxy; 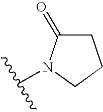
which is optionally substituted by C₁₋₄ alkyl, C₁₋₄ alkoxy, or F; 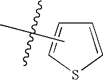
which is optionally substituted by C₁₋₄ alkyl or halogen; 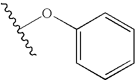
which is optionally substituted by halogen, OH, C₁₋₄ alkoxy, or C₁₋₄ alkyl which is optionally substituted by F;
2) 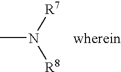
R⁷ represents H or C₁₋₄ alkyl;
R⁸ represents
H;
—(CH₂)ₕ—O—(CH₂)ᵢ—OR³⁶ wherein
R³⁶ represents H or C₁₋₄ alkyl which is optionally substituted by F, and
subscripts h and i are independently 1, 2, or 3; 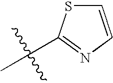
which is optionally substituted by halogen or by C₁₋₄ alkyl which is optionally substituted by F; or
C₁₋₆ alkyl which is optionally substituted by up to three substituents selected from:
OH;
C₁₋₄ alkoxy; 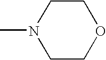
which is optionally substituted by F or C₁₋₄ alkyl; 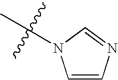
which is optionally substituted by halogen or C₁₋₄ alkyl which is optionally substituted by F; 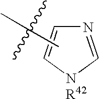
wherein the ring is optionally substituted by halogen, and R⁴² represents H or C₁₋₄ alkyl which is optionally substituted by F; 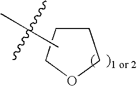
which is optionally substituted by F or C₁₋₄ alkyl; and 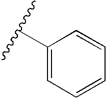
which is optionally substituted by halogen, C₁₋₄ alkoxy, or C₁₋₄ alkyl which is optionally substituted by F;
7) 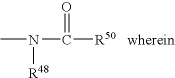
R⁴⁸ represents H; and
R⁵⁰ represents
—(CH₂)ⱼ—O—(CH₂)ₖ—R^{50a} wherein
R^{50a} represents OH or C₁₋₄ alkoxy; and
subscripts j and k are independently 1, 2, or 3;
or R⁵⁰ represents
C₁₋₄ alkyl optionally substituted by C₁₋₂ alkoxy or C₁₋₄ acyloxy;
8) 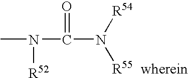
R⁵² represents H or C₁₋₂ alkyl;
R⁵⁴ represents H or C₁₋₄ alkyl; and
R⁵⁵ represents H; C₁₋₄ alkyl optionally substituted by F or C₁₋₄ alkoxy; 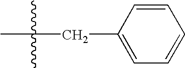
wherein the ring is optionally substituted by C₁₋₄ alkyl, C₁₋₄ alkoxy, or OH; or R⁵⁵ represents 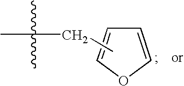
R⁵⁴ and R⁵⁵ are optionally joined and together with the N atom to which they are attached form a 5- or 6-membered saturated heterocycle selected from pyrrolidinyl, morpholinyl, thiomorpholinyl, and piperizinyl optionally substituted on N with C₁₋₄ alkyl;
9) 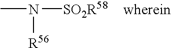
R⁵⁶ represents H or C₁₋₂ alkyl; and
R⁵⁸ represents C₁₋₄ alkyl which is optionally substituted by F; or
represents phenyl which is optionally substituted by halogen, C₁₋₄ alkyl, or C₁₋₄ alkoxy.

4. The compound of claim 2 wherein
Z represents CH;
R³ represents
1) 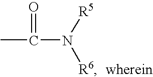
R⁵ represents H or C₁₋₄ alkyl which may optionally be substituted by OH or C₁₋₄ alkoxy; and
R⁶ represents
H;
C₃₋₆ cycloalkyl;
—(CH₂)_{f}—O—(CH₂)_{g}—OR¹⁰ wherein R¹⁰ represents H or C₁₋₄ alkyl which is optionally substituted by F, and the subscripts f and g each independently represents 1, 2, or 3;
C₁₋₆ alkyl, which is optionally fluorinated up to perfluoro, or independently substituted by up to three substituents selected from:
—SO₂R¹⁴ in which R¹⁴ represents C₁₋₅ alkyl which is optionally substituted by C₁₋₄ alkoxy or F; 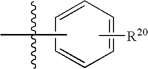
wherein R²⁰ represents from 0 to 4 optional substituents independently selected from C₁₋₄ alkyl, OH, C₁₋₄ alkoxy, halogen, NO₂, CN, and morpholino; 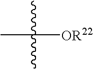
wherein R²² represents H or C₁₋₅ alkyl which is optionally substituted by F, 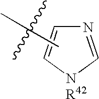
in which the ring is optionally substituted by C₁₋₄ alkyl or halogen, and R²⁴ represents H or C₁₋₄ alkyl which is optionally substituted by F; 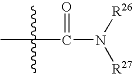
wherein R²⁶ and R²⁷ independently represent H or C₁₋₂ alkyl optionally substituted by F; 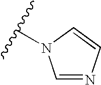
which is optionally substituted by halogen or by C₁₋₄ alkyl optionally substituted by F;
—CO₂R²⁸ wherein R²⁸ represents C₁₋₄ alkyl; 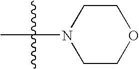
optionally substituted by F or C₁₋₄ alkyl; 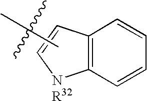
wherein R³² represents H or C₁₋₄ alkyl optionally substituted by F, and the phenyl ring is optionally substituted by C₁₋₄ alkyl, C₁₋₄ alkoxy, or halogen; 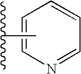
which is optionally substituted by C₁₋₄ alkyl or C₁₋₄ alkoxy; 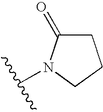
which is optionally substituted by C₁₋₄ alkyl, C₁₋₄ alkoxy, or F; 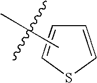
which is optionally substituted by C₁₋₄ alkyl or halogen; 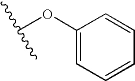
which is optionally substituted by halogen, OH, C₁₋₄ alkoxy, or C₁₋₄ alkyl which is optionally substituted by F;
8) 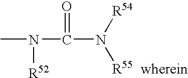
R⁵² represents H or C₁₋₂ alkyl;
R⁵⁴ represents H or C₁₋₄ alkyl; and
R⁵⁵ represents H; C₁₋₄ alkyl optionally substituted by F or C₁₋₄ alkoxy; 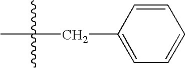
wherein the ring is optionally substituted by C₁₋₄ alkyl, C₁₋₄ alkoxy, or OH; or R⁵⁵ represents 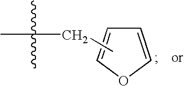
R⁵⁴ and R⁵⁵ are optionally joined and together with the N atom to which they are attached form a 5- or 6-membered saturated heterocycle selected from pyrrolidinyl, morpholinyl, thiomorpholinyl, and piperizinyl optionally substituted on N with C₁₋₄ alkyl;
9) 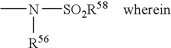
R⁵⁶ represents H or C₁₋₂ alkyl; and
R⁵⁸ represents C₁₋₄ alkyl which is optionally substituted by F; or represents phenyl which is optionally substituted by halogen, C₁₋₄ alkyl, or C₁₋₄ alkoxy.

5. A compound having the formula (II) 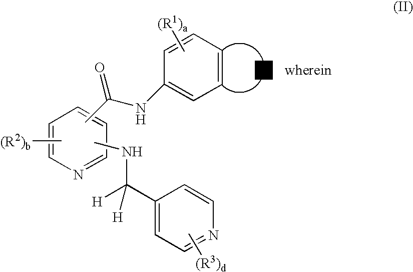
R¹ represents C₁₋₄ alkyl or halogen;
a is 0, 1 or 2;
R² represents C₁₋₄ alkyl, C₁₋₄ alkoxy, or halogen;
b is 0, 1, 2, or 3;
R³ represents —C(O)NR⁵R⁶; —NR⁷R⁸; —CN; -halogen; —C₁₋₄ alkyl; or 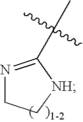
d is 0 or 1;
A represents 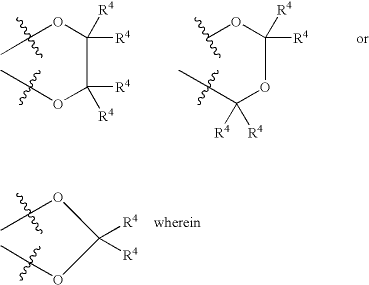
R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring;
R⁵ and R⁶ each independently represents H, C₁₋₄ alkyl, or —C₁₋₄-alkyl-C₁₋₂-alkoxy;
R⁷ and R⁸ each independently represents H or C₁₋₄ alkyl;
with the proviso that any R³ group is located adjacent to the ring nitrogen atom, and the amido and amino side chains on the central pyridine ring are located adjacent to each other;
or a pharmaceutically acceptable salt or stereoisomer thereof.

6. The compound of claim 5 wherein
R² represents C₁₋₄ alkyl or halogen; and
R³ represents —C(O)NR⁵R⁶, —NR⁷R⁸, or —C₁₋₄ alkyl.

7. A compound having formula (III) or formula (IV) 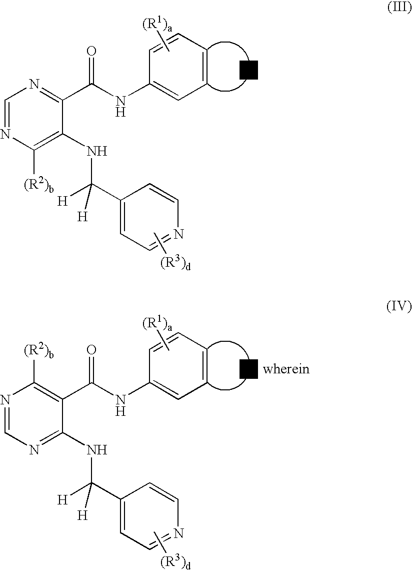
R¹ represents C₁₋₄ alkyl or halogen;
a is 0, 1 or 2;
R² represents C₁₋₄ alkyl, C₁₋₄ alkoxy, or halogen;
b is 0 or 1;
R³ represents —C(O)NR⁵R⁶; —NR⁷R⁸; —CN; -halogen; —C₁₋₄ alkyl; or 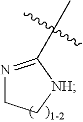
d is 0 or 1;
A represents 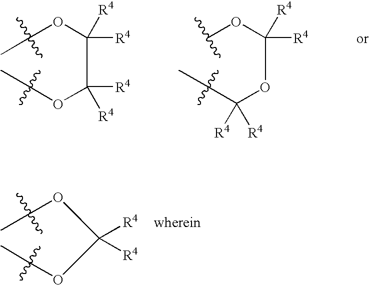
R⁴ represents halogen, CF₃, or H, provided that the maximum number of CF₃ groups on any A is 2, and the maximum number of hydrogens on A is 2 for the A groups which together with the carbon atoms to which they are attached form 6-membered rings, and the maximum number of hydrogens on A is 1 for the A group which together with the carbon atoms to which it is attached forms a 5-membered ring;
R⁵ and R⁶ each independently represents H, C₁₋₄ alkyl, or —C₁₋₄-alkyl-C₁₋₂-alkoxy;
R⁷ and R⁸ each independently represents H or C₁₋₄ alkyl;
with the further proviso that any R³ group is located adjacent to the ring nitrogen atom;
or a pharmaceutically acceptable salt or stereoisomer thereof.

8. The compound of claim 7
wherein
R² represents C₁₋₄ alkyl or halogen; and
R³ represents —C(O)NR⁵R⁶, —NR⁷R⁸, or —C₁₋₄ alkyl.

9. A compound selected from the following table of compounds:

| Example | |
|---|---|
| No. | Compound name |
| 3 | N-methyl-4-{[(2-{[(2,2,3,3-tetrafluoro-2,3-dihydro-1,4- |
| | benzodioxin-6-yl)amino]carbonyl}phenyl)amino]methyl}- |
| | pyridine-2-carboxamide |
| 4 | N-methyl-4-{[(2-{[(2,2,4,4-tetrafluoro-4H-1,3-benzodioxin- |
| | 6-yl)amino]carbonyl}phenyl)amino]methyl}pyridine-2- |
| | carboxamide |
| 24 | 4-{[(2-{[(2,2-difluoro-1,3-benzodioxol-5- |
| | yl)amino]carbonyl}phenyl)amino]methyl}-N-methylpyridine- |
| | 2-carboxamide |
| 31 | 4-{[(2-{[(2,2-difluoro-1,3-benzodioxol-5-yl)amino]- |
| | carbonyl}-4-fluorophenyl)amino]methyl}-N-methylpyridine- |
| | 2-carboxamide |
| 32 | 4-{[(5-fluoro-2-{[(2,2,3,3-tetrafluoro-2,3-dihydro-1,4- |
| | benzodioxin-6-yl)amino]carbonyl}phenyl)amino]methyl}-N- |
| | methylpyridine-2-carboxamide |
| 33 | 4-{[(2-{[(2,2-difluoro-1,3-benzodioxol-5-yl)amino]- |
| | carbonyl}-5-fluorophenyl)amino]methyl}-N-methylpyridine- |
| | 2-carboxamide |
| 34 | 4-{[(4-fluoro-2-{[(2,2,3,3-tetrafluoro-2,3-dihydro-1,4- |
| | benzodioxin-6-yl)amino]carbonyl}phenyl)amino]methyl}-N- |
| | methylpyridine-2-carboxamide |
| 35 | 4-{[(4-fluoro-2-{[(2,2,3,3-tetrafluoro-2,3-dihydro-1,4- |
| | benzodioxin-6-yl)amino]carbonyl}phenyl)amino]methyl}-N- |
| | methylpyridine-2-carboxamide |
| 37 | 4-{[(4,5-difluoro-2-{[(2,2,3,3-tetrafluoro-2,3-dihydro-1,4- |
| | benzodioxin-6-yl)amino]carbonyl}phenyl)amino]methyl}-N- |
| | methylpyridine-2-carboxamide |
| 43 | 4-{[(4,5-difluoro-2-{[(2,2,3,3-tetrafluoro-2,3-dihydro-1,4- |
| | benzodioxin-6-yl)amino]carbonyl}phenyl)amino]methyl}-N- |
| | ethylpyridine-2-carboxamide |
| 44 | N-ethyl-4-{[(2-{[(2,2,3,3-tetrafluoro-2,3-dihydro-1,4- |
| | benzodioxin-6-yl)amino]carbonyl}phenyl)amino]- |
| | methyl}pyridine-2-carboxamide |
| 45 | 4-{[(2-{[(2,2-difluoro-1,3-benzodioxol-5-yl)amino]- |
| | carbonyl}phenyl)amino]methyl}-N-ethylpyridine-2- |
| | carboxamide |
| 46 | 4-{[(2-{[(2,2-difluoro-1,3-benzodioxol-5-yl)amino]- |
| | carbonyl}-4-fluorophenyl)amino]methyl}-N-ethylpyridine-2- |
| | carboxamide |
| 47 | 4-{[(2-{[(2,2-difluoro-1,3-benzodioxol-5-yl)amino]- |
| | carbonyl}-5-fluorophenyl)amino]methyl}-N-ethylpyridine-2- |
| | carboxamide |
| 49 | N-(2,2-difluoro-1,3-benzodioxol-5-yl)-2-({[2- |
| | (methylamino)pyrimidin-4-yl]methyl}amino)benzamide |
| 51 | 2-({[2-(methylamino)pyrimidin-4-yl]methyl}amino)-N- |
| | (2,2,3,3-tetrafluoro-2,3-dihydro-1,4-benzodioxin-6- |
| | yl)benzamide |
| 52 | 4-{[(2-{[(2,2-difluoro-1,3-benzodioxol-5- |
| | yl)amino]carbonyl}phenyl)amino]methyl}-N-(2- |
| | methoxyethyl)pyridine-2-carboxamide |
| 53 | 4-{[(2-{[(2,2-difluoro-1,3-benzodioxol-5- |
| | yl)amino]carbonyl}-4-fluorophenyl)amino]methyl}-N-(2- |
| | methoxyethyl)pyridine-2-carboxamide |
| 74 | N-cyclopropyl-4-{[(2-{[(2,2-difluoro-1,3-benzodioxol-5- |
| | yl)amino]carbonyl}phenyl)amino]methyl}pyridine-2- |
| | carboxamide |
| 139 | N-(2,2-difluoro-1,3-benzodioxol-5-yl)-2-({[2- |
| | (methylamino)pyridin-4-yl]methyl}amino)benzamide |
| | trifluoroacetate |
| 162 | N-(2,2-difluoro-1,3-benzodioxol-5-yl)-2-{[(2- |
| | {[(ethylamino)carbonyl]amino}pyridin-4- |
| | yl)methyl]amino}benzamide |
| 165 | N-(2,2-difluoro-1,3-benzodioxol-5-yl)-2-{[(2- |
| | {[(methylamino)carbonyl]amino}pyridin-4- |
| | yl)methyl]amino}benzamide |
| 169 | N-(2,2-difluoro-1,3-benzodioxol-5-yl)-2-{[(2- |
| | {[(propylamino)carbonyl]amino}pyridin-4- |
| | yl)methyl]amino}benzamide |
| 170 | 2-{[(2-{[(butylamino)carbonyl]amino}pyridin-4- |
| | yl)methyl]amino}-N-(2,2-difluoro-1,3-benzodioxol-5- |
| | yl)benzamide |
| 175 | N-(2,2-difluoro-1,3-benzodioxol-5-yl)-2-[({2- |
| | [(methylsulfonyl)amino]pyridin-4- |
| | yl}methyl)amino]benzamide |

10. A pharmaceutical composition which comprises a compound of claim 1 plus a pharmaceutically acceptable carrier.

11. A method of treating cancer comprising administering to a subject in need thereof an effective amount of a compound of claim 1.
